(19) United States
(12) Patent Application Publication
(10) Pub. No.: US 2022/0160849 A1
(43) Pub. Date: May 26, 2022

(54) CANCER VACCINE FORMULATION

(71) Applicants: MIE UNIVERSITY, Tsu-shi, Mie (JP); KYOTO UNIVERSITY, Kyoto-shi, Kyoto (JP); CHUGAI SEIYAKU KABUSHIKI KAISHA, Tokyo (JP)

(72) Inventors: Hiroshi SHIKU, Kurimamachiya-cho, Tsu-shi, Mie (JP); Kazunari AKIYOSHI, Kyoto-shi, Kyoto (JP); Tai HIRAKURA, Gotemba-shi, Shizuoka (JP); Tsuyoshi SHIMOBOJI, Gotemba-shi, Shizuoka (JP); Takashi NAKAI, Gotemba-shi, Shizuoka (JP); Sayan CHUANOI, Gotemba-shi, Shizuoka (JP); Hideyuki TOGAWA, Gotemba-shi, Shizuoka (JP)

(21) Appl. No.: 17/425,469

(22) PCT Filed: Jan. 29, 2020
(86) PCT No.: PCT/JP2020/003076

§ 371 Date: Jul. 23, 2021

(30) Foreign Application Priority Data
Jan. 29, 2019 (JP) 2019-013556

(51) Int. Cl. A61K 39/00; A61K 39/395; A61P 35/00

(52) U.S. Cl. CPC A61K 39/0011; A61K 39/3955; A61K 2039/627; A61K 2039/6087; A61P 35/00

(57) ABSTRACT

The present invention provides a vaccine formulation for use in the prevention and/or treatment of a cancer, comprising a complex of a hyaluronic acid derivative having an introduced hydrophobic group, and an antigen.

### TECHNICAL FIELD

The present invention relates to a vaccine formulation for use in the prevention and/or treatment of a cancer, comprising a complex of a hyaluronic acid derivative having an introduced hydrophobic group, and an antigen.

### BACKGROUND ART

A vaccine formulation for use in the prevention and/or treatment of a cancer has been reported which comprises a hydrophobized polysaccharide such as cholesterol-bearing pullulan (CHP) or cholesterol-bearing mannan (CMP), and an antigen (Patent Literature 1). Also, a vaccine formulation for cancer treatment has been reported which comprises a complex of a hydrophobized polysaccharide such as CHP, and a synthesized long-chain peptide antigen having a plurality of T cell recognition epitopes, and an immunopotentiating agent (Patent Literatures 2 and 3).

These vaccine formulations are based on the principles that antigens phagocytized by antigen-presenting cells are cleaved into peptides of various lengths by intracellular proteasome, protease, or peptidase and loaded as antigenic epitope peptides onto major histocompatibility complex (MHC) class I molecules or MHC class II molecules on the cell surface to form complexes, which are in turn specifically recognized by cytotoxic T cells (CTL, CD8⁺) or helper T cells (CD4⁺) so that these cells are activated (Non Patent Literature 1).

Meanwhile, it has been reported that a hyaluronic acid derivative obtained by introducing a group having a cholesteryl group as a hydrophobic group to hyaluronic acid forms fine particles by association in water and forms a complex with a drug (Patent Literature 4). It has also been reported that a hyaluronic acid derivative obtained by converting a carboxyl group of the glucuronic acid moiety of hyaluronic acid into an amide group through reaction with a particular amino acid, and further introducing a steryl group which is a hydrophobic group to remaining carboxyl groups has both characteristics of biodegradability and retention in blood, and a complex of the hyaluronic acid derivative and a drug has characteristics favorable as a pharmaceutical composition (Patent Literature 5).

### CITATION LIST

### Patent Literature

[Patent Literature 1] International Publication No. WO 1998/009650
[Patent Literature 2] International Publication No. WO 2013/031882
[Patent Literature 3] International Publication No. WO 2015/050158
[Patent Literature 4] International Publication No. WO 2010/053140
[Patent Literature 5] International Publication No. WO 2014/038641

### Non Patent Literature

[Non Patent Literature 1] ACS Nano Vol. 8, p. 9209-9218, 2014

### SUMMARY OF INVENTION

### Technical Problem

There is a demand for a vaccine formulation having a higher function than that of already reported vaccine formulations containing CHP. Particularly, it is desirable to develop a cancer vaccine formulation having characteristics of markedly inducing antigen-specific cytotoxic T cells (CTL) or a high antitumor effect.

### Solution to Problem

The present inventor has conducted diligent studies to solve these problems and consequently completed the present invention by finding that a vaccine formulation comprising a complex of a hyaluronic acid derivative having an introduced hydrophobic group, and an antigen markedly induces antigen-specific CTL and has high antitumor activity.

Specifically, the present invention relates to a complex that is formed by enclosing an antigen through spontaneous association in an aqueous solution, improves the accumulation of the antigen to lymph node, and markedly induces antigen-specific CTL, a complex having high antitumor activity, a vaccine formulation comprising the complex, and production methods therefor. The present invention also relates to a complex that is formed by enclosing an antigen through better dispersion in water, improves the accumulation of the antigen to lymph node, and markedly induces antigen-specific CTL, a vaccine formulation comprising the complex, and production methods therefor.

In one aspect, the present invention provides a vaccine formulation for use in the prevention and/or treatment of a cancer according to any of [1] to [13] and [15] to [27], a complex for use in the vaccine formulation according to any of [14] and [28], and a method for producing the complex according to [29] as follows.

[1] A vaccine formulation for use in the prevention or treatment of a cancer, comprising a hyaluronic acid derivative having an introduced hydrophobic group, and an antigen, wherein

the hyaluronic acid derivative having an introduced hydrophobic group is the following:

a hyaluronic acid derivative comprising at least one repeating unit represented by the formula (I):

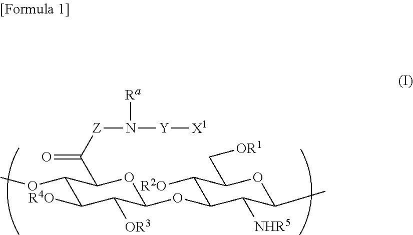

wherein R¹, R², R³, and R⁴ are each independently selected from a hydrogen atom, C₁₋₆ alkyl, formyl and C₁₋₆ alkylcarbonyl;

R⁵ is a hydrogen atom, formyl, or C₁₋₆ alkylcarbonyl;

Z represents a direct bond, or a peptide linker having 2 to 30 arbitrary amino acid residues;

X¹ is a hydrophobic group selected from groups represented by the following formulas:

—NR^{b}—R,

—NR^{b}—COO—R,

—NR^{b}—CO—R,

—NR^{b}—CO—NR^{c}—R,

—COO—R,

—O—COO—R,

—S—R,

—CO—Y^{a}—S—R,

—O—CO—Y^{b}—S—R,

—NR^{b}—CO—Y^{b}—S—R, and

—S—S—R;

R^{a}, R^{b} and R^{c} are each independently selected from a hydrogen atom, C₁₋₂₀ alkyl, amino-C₂₋₂₀ alkyl and hydroxy-C₂₋₂₀ alkyl, wherein in the alkyl moiety of each of the groups, 1 to 3 groups each independently selected from —O— and —NR^{f}— are optionally inserted;

R^{f} is selected from a hydrogen atom, C₁₋₁₂ alkyl, amino-C₂₋₁₂ alkyl and hydroxy-C₂₋₁₂ alkyl, wherein in the alkyl moiety of each of the groups, 1 or 2 groups each independently selected from —O— and —NH— are optionally inserted;

R is a steryl group;

Y is C₂₋₃₀ alkylene, or —(CH₂CH₂O)ₘ—CH₂CH₂—, wherein in the alkylene, 1 to 5 groups each independently selected from —O—, —NR^{g}— and —S—S— are optionally inserted;

R^{g} is selected from a hydrogen atom, C₁₋₂₀ alkyl, amino-C₂₋₂₀ alkyl or hydroxy-C₂₋₂₀ alkyl, wherein in the alkyl moiety of each of the groups, 1 to 3 groups each independently selected from —O— and —NH— are optionally inserted;

Y^{a} is C₁₋₅ alkylene;

Y^{b} is C₂₋₈ alkylene or C₂₋₈ alkenylene; and

m is an integer selected from 1 to 100; or

a hyaluronic acid derivative comprising a repeating unit represented by the formula (II):

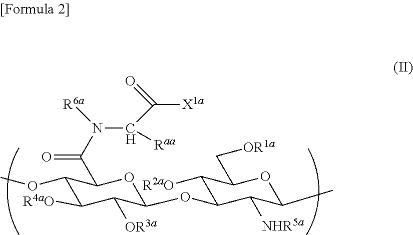

wherein R^{1a}, R^{2a}, R^{3a}, and R^{4a} are each independently selected from a hydrogen atom, C₁₋₆ alkyl, formyl, and C₁₋₆ alkylcarbonyl;

R^{5a} is a hydrogen atom, for C₁₋₆ alkylcarbonyl;

X^{1a} is hydroxy, —O⁻Q⁺, C₁₋₆ alkoxy, —NR⁷R⁸, or —NR⁹—Z¹—Z²;

Q⁺ represents a counter cation;

R^{6a}, R⁷, R⁸, and R⁹ are each independently selected from a hydrogen atom, and C₁₋₆ alkyl;

R^{aa} is a hydrogen atom, or C₁₋₆ alkyl, wherein the alkyl is optionally substituted by one or more groups each independently selected from hydroxy, carboxy, carbamoyl, C₁₋₆ alkylthio, aryl, and heteroaryl, wherein the aryl is optionally substituted by one or more hydroxy groups;

Z¹ is C₂₋₃₀ alkylene, or —(CH₂CH₂O)ₘ—CH₂CH₂—, wherein in the alkylene, 1 to 5 groups each independently selected from-O—, —NR^{ga}— and —S—S— are optionally inserted, and ma is an integer selected from 1 to 100;

Z² is selected from groups represented by the following formulas:

—NR^{ba}—Z³,

—NR^{ba}—COO—Z³,

—NR^{ba}—CO—Z³,

—NR^{ba}—CO—NR^{ca}—Z³,

—COO—Z³,

—CO—NR^{ca}—Z³,

—O—CO—NR^{ca}—Z³,

—O—COO—Z³,

—S—Z³,

—CO—Z^{a}—S—Z³,

—O—CO—Z^{b}—S—Z³,

—NR^{ba}—CO—Z^{b}—S—Z³, and

—S—S—Z³;

R^{ba} and R^{ca} are each independently selected from a hydrogen atom, C₁₋₂₀ alkyl, amino-C₂₋₂₀ alkyl and hydroxy-C₂₋₂₀ alkyl, wherein in the alkyl moiety of each of the groups, 1 to 3 groups each independently selected from —O— and —NR^{fa}— are optionally inserted;

R^{fa} is independently selected from a hydrogen atom, C₁₋₁₂ alkyl, amino-C₂₋₁₂ alkyl and hydroxy-C₂₋₂₀ alkyl, wherein in the alkyl moiety of each of the groups, 1 or 2 groups each independently selected from —O— and —NH— are optionally inserted;

R^{ga} is independently selected from a hydrogen atom, C₁₋₂₀ alkyl, amino-C₂₋₂₀ alkyl and hydroxy-C₂₋₂₀ alkyl, wherein in the alkyl moiety of each of the groups, 1 to 3 groups each independently selected from —O— and —NH— are optionally inserted;

Z³ is a steryl group;

Z^{a} is C₁₋₅ alkylene; and

Z^{b} is C₂₋₈ alkylene or C₂₋₈ alkenylene,

the hyaluronic acid derivative, when not comprising a repeating unit represented by the formula (II) wherein X^{1a} is —NR⁹—Z¹—Z², further comprising a repeating unit represented by the formula (III):

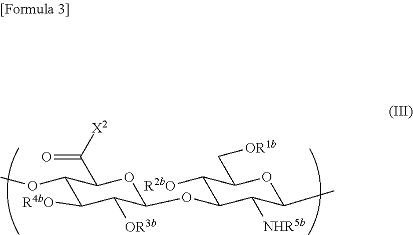

wherein R^{1b}, R^{2b}, R^{3b} and R^{4b} are each independently selected from a hydrogen atom, C₁₋₆ alkyl, formyl, and C₁₋₆ alkylcarbonyl;

R^{5b} is a hydrogen atom, formyl, or C₁₋₆ alkylcarbonyl; and

X² is —NR⁹—Z¹—Z², wherein R⁹, Z¹, and Z² are as already defined.

[2] The vaccine formulation according to [1], wherein the hyaluronic acid derivative having an introduced hydrophobic group further comprises a repeating unit represented by the formula (IIIc):

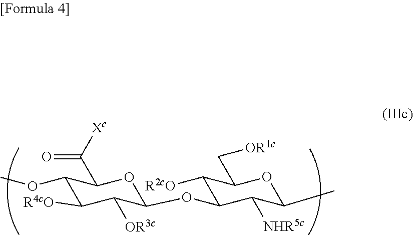

wherein R^{1c}, R^{2c}, R^{3c} and R^{4c} are each independently selected from a hydrogen atom, C₁₋₆ alkyl, formyl and C₁₋₆ alkylcarbonyl;

R^{5c} is selected from a hydrogen atom, formyl and C₁₋₆ alkylcarbonyl; and

X^{c} is selected from hydroxy and —O⁻Q⁺, wherein Q⁺ represents a counter cation.

[3] The vaccine formulation according to [1] or [2], wherein the vaccine formulation comprises a hyaluronic acid derivative comprising a repeating unit represented by the formula (I), wherein a ratio of the repeating unit represented by the formula (I) to disaccharide repeating units present is 5 to 50%.

[4] The vaccine formulation according to [1] or [2], wherein the vaccine formulation comprises a hyaluronic acid derivative comprising a repeating unit represented by the formula (II), wherein a ratio of a disaccharide unit comprising the group —NR⁹—Z¹—Z² to disaccharide repeating units present is 5 to 50%.

[5] The vaccine formulation according to any of [1] to [3], wherein the vaccine formulation comprises a hyaluronic acid derivative comprising a repeating unit represented by the formula (I), wherein Z is a direct bond, Y is C₂₋₁₀ alkylene, X¹ is —NH—COO—R, and R is a cholesteryl group.

[6] The vaccine formulation according to any of [1], [2] and [4], wherein the vaccine formulation comprises a hyaluronic acid derivative comprising a repeating unit represented by the formula (II), wherein Z¹ is C₂₋₁₀ alkylene, Z² is —NH—COO—Z³, and Z³ is a cholesteryl group.

[7] The vaccine formulation according to any of [1] to [6], wherein the hyaluronic acid derivative is produced using hyaluronic acid composed only of a disaccharide unit represented by the formula (IIIc) defined in claim 2, wherein when all of R^{1c}, R^{2c}, R^{3c}, and R^{4c} are hydrogen atoms, R^{5c} is acetyl, and X^{c} is —O⁻Na⁺, a weight-average molecular weight is 5 kilodaltons to 200 kilodaltons.

[8] The vaccine formulation according to any of [1] to [7], wherein the hyaluronic acid derivative and the antigen form a complex.

[9] The vaccine formulation according to any of [1] to [8] for administration in combination with at least one type of adjuvant.

[10] The vaccine formulation according to any of [1] to [9], wherein the antigen is an antigenic peptide or an antigenic protein.

[11] The vaccine formulation according to [10], wherein the antigenic peptide comprises two or more CD8-positive cytotoxic T cell recognition epitopes or CD4-positive helper T cell recognition epitopes.

[12] The vaccine formulation according to [11], wherein the antigenic peptide has an amino acid linker between the epitopes.

[13] The vaccine formulation according to any of [1] to [12] for administration in combination with at least one type of antibody for use in cancer treatment.

[14] A complex formed from a hyaluronic acid derivative comprising a repeating unit represented by the formula (I) or the formula (II) according to any of [1] to [7], and an antigen that is used in a vaccine for use in the prevention or treatment of a cancer.

[15] The vaccine formulation according to any of [1] to [3], [5] and [7] to [13], wherein the vaccine formulation comprises a hyaluronic acid derivative comprising at least one repeating unit represented by the formula (I), wherein Y is —(CH₂)ₙ₁— or —(CH₂CH₂O)ₘ₁—CH₂CH₂—, n1 is an integer of 2 to 15, and m1 is an integer of 1 to 4.

[16] The vaccine formulation according to any of [1] to [3], [5], [7] to [13] and [15], wherein the vaccine formulation comprises a hyaluronic acid derivative comprising at least one repeating unit represented by the formula (I), wherein Y is —(CH₂)ₙ₁—.

[17] The vaccine formulation according to any of [1] to [3], [5], [7] to [13], [15], and [16], wherein the vaccine formulation comprises a hyaluronic acid derivative comprising at least one repeating unit represented by the formula (I), wherein Y is —(CH₂)ₙ₁, and n1 is 2, 6, 8, or 12.

[18] The vaccine formulation according to any of [1] to [13] and [15] to [17] for administration in combination with at least one type of adjuvant, wherein the adjuvant is i) a substance activating an innate immune receptor (pattern recognition receptor), ii) an antigen-presenting cell-stimulating substance, or iii) a substance having an effect of inhibiting the acquirement of immunosuppressive activity by antigen-presenting cells.

[19] The vaccine formulation according to any of [1] to [13] and [15] to [18] for administration in combination with at least one type of adjuvant, wherein the adjuvant is CpG oligo DNA, polyIC, QuilA, QS21, Sting, monophosphoryl lipid, R848, imiquimod, or MPL.

[20] The vaccine formulation according to any of [1] to [13] and [15] to [19], wherein the antigen is an antigenic peptide, and the antigenic peptide comprises one or more each of CD8-positive cytotoxic T cell recognition epitopes and CD4-positive helper T cell recognition epitopes.

[21] The vaccine formulation according to any of [1] to [13] and [15] to [20], wherein the antigen is an antigenic peptide, and the number of amino acid residues in the antigenic peptide is 8 to 120.

[22] The vaccine formulation according to any of [1] to [13] and [15] to [21], wherein the antigen is an antigenic peptide, and the number of amino acid residues in the antigenic peptide is 8 to 50.

[23] The vaccine formulation according to any of [1] to [13] and [15] to [22], wherein the antigen is an antigenic peptide, and the number of amino acid residues in the antigenic peptide is 16 to 80.

[24] The vaccine formulation according to any of [1] to [13] and [15] to [23], wherein the antigen is an antigenic peptide, and the number of amino acid residues in the antigenic peptide is 23 to 60.

[25] The vaccine formulation according to any of [1] to [13] and [15] to [24] for administration in combination with at least one type of antibody for use in cancer treatment, wherein the antibody is an antibody inhibiting an immunosuppressive signal from tumor, or at least one type of antibody activating a costimulatory signal of immunocytes.

[26] The vaccine formulation according to any of [1] to [13] and [15] to [25] for administration in combination with at least one type of antibody for use in cancer treatment, wherein the antibody is an anti-CTLA4 antibody, an anti-PD1 antibody, an anti-PDL1 antibody, an anti-OX40 antibody, or an anti-4-1BB antibody.

[27] The vaccine formulation according to any of [1] to [13] and [15] to [26] for administration in combination with at least one type of antibody for use in cancer treatment, wherein the antibody is an anti-PDL1 antibody.

[28] A complex formed from a hyaluronic acid derivative comprising a repeating unit represented by the formula (I) or the formula (II) according to any of [15] to [17], and an antigen that is used in a vaccine for use in the prevention or treatment of a cancer.

[29] A method for producing a complex of a hyaluronic acid derivative having an introduced hydrophobic group, and an antigen, comprising the step of mixing a hyaluronic acid derivative comprising a repeating unit represented by the formula (I) or the formula (II) according to any of [1] to [7] and [15] to [17] with an antigenic peptide that is used in a vaccine for use in the prevention or treatment of a cancer in a solution.

In another aspect, the present invention provides a method for preventing and/or treating a cancer according to any of [30] to [46], and use according to any of [47] to [63] as follows.

[30] A method for preventing and/or treating a cancer, comprising administering a vaccine formulation comprising a hyaluronic acid derivative comprising a repeating unit represented by the formula (I) or the formula (II), and an antigen according to any of [1] to [7] and [15] to [17] to a subject.

[31] The method according to [30], wherein the hyaluronic acid derivative and the antigen form a complex.

[32] The method according to [30], wherein the vaccine formulation is administered in combination with at least one type of adjuvant.

[33] The method according to [30], wherein the antigen is an antigenic peptide or an antigenic protein.

[34] The method according to [33], wherein the antigenic peptide comprises two or more CD8-positive cytotoxic T cell recognition epitopes or CD4-positive helper T cell recognition epitopes.

[35] The method according to [34], wherein the antigenic peptide has an amino acid linker between epitopes.

[36] The method according to [30], wherein the vaccine formulation is administered in combination with at least one type of antibody for use in cancer treatment.

[37] The method according to [30], wherein the vaccine formulation is administered in combination with at least one type of adjuvant, and the adjuvant is i) a substance activating an innate immune receptor (pattern recognition receptor), ii) an antigen-presenting cell-stimulating substance, or iii) a substance having an effect of inhibiting the acquirement of immunosuppressive activity by antigen-presenting cells.

[38] The method according to [30], wherein the vaccine formulation is administered in combination with at least one type of adjuvant, and the adjuvant is CpG oligo DNA, polyIC, QuilA, QS21, Sting, monophosphoryl lipid, R848, imiquimod, or MPL.

[39] The method according to [30], wherein the antigen is an antigenic peptide, and the antigenic peptide comprises one or more each of CD8-positive cytotoxic T cell recognition epitopes and CD4-positive helper T cell recognition epitopes.

[40] The method according to [30], wherein the antigen is an antigenic peptide, and the number of amino acid residues in the antigenic peptide is 8 to 120.

[41] The method according to [30], wherein the antigen is an antigenic peptide, and the number of amino acid residues in the antigenic peptide is 8 to 50.

[42] The method according to [30], wherein the antigen is an antigenic peptide, and the number of amino acid residues in the antigenic peptide is 16 to 80.

[43] The method according to [30], wherein the antigen is an antigenic peptide, and the number of amino acid residues in the antigenic peptide is 23 to 60.

[44] The method according to [30], wherein the vaccine formulation is administered in combination with at least one type of antibody for use in cancer treatment, and the antibody is an antibody inhibiting an immunosuppressive signal from tumor, or at least one type of antibody activating a costimulatory signal of immunocytes.

[45] The method according to [30], wherein the vaccine formulation is administered in combination with at least one type of antibody for use in cancer treatment, and the antibody is an anti-CTLA4 antibody, an anti-PD1 antibody, an anti-PDL1 antibody, an anti-OX40 antibody, or an anti-4-1BB antibody.

[46] The method according to [30], wherein the vaccine formulation is administered in combination with at least one type of antibody for use in cancer treatment, and the antibody is an anti-PDL1 antibody.

[47] Use of a hyaluronic acid derivative comprising a repeating unit represented by the formula (I) or the formula (II), and an antigen according to any of [1] to [7] and [15] to [17] in the production of a vaccine formulation for use in the prevention or treatment of a cancer.

[48] Use according to [47], wherein the hyaluronic acid derivative and the antigen form a complex.

[49] Use according to [47], wherein the vaccine formulation is administered in combination with at least one type of adjuvant.

[50] Use according to [47], wherein the antigen is an antigenic peptide or an antigenic protein.

[51] Use according to [50], wherein the antigenic peptide comprises two or more CD8-positive cytotoxic T cell recognition epitopes or CD4-positive helper T cell recognition epitopes.

[52] Use according to [51], wherein the antigenic peptide has an amino acid linker between epitopes.

[53] Use according to [47], wherein the vaccine formulation is administered in combination with at least one type of antibody for use in cancer treatment.

[54] Use according to [47], wherein the vaccine formulation is administered in combination with at least one type of adjuvant, and the adjuvant is i) a substance activating an innate immune receptor (pattern recognition receptor), ii) an antigen-presenting cell-stimulating substance, or iii) a substance having an effect of inhibiting the acquirement of immunosuppressive activity by antigen-presenting cells.

[55] Use according to [47], wherein the vaccine formulation is administered in combination with at least one type of adjuvant, and the adjuvant is CpG oligo DNA, polyIC, QuilA, QS21, Sting, monophosphoryl lipid, R848, imiquimod, or MPL.

[56] Use according to [47], wherein the antigen is an antigenic peptide, and the antigenic peptide comprises one or more each of CD8-positive cytotoxic T cell recognition epitopes and CD4-positive helper T cell recognition epitopes.

[57] Use according to [47], wherein the antigen is an antigenic peptide, and the number of amino acid residues in the antigenic peptide is 8 to 120.

[58] Use according to [47], wherein the antigen is an antigenic peptide, and the number of amino acid residues in the antigenic peptide is 8 to 50.

[59] Use according to [147], wherein the antigen is an antigenic peptide, and the number of amino acid residues in the antigenic peptide is 16 to 80.

[60] Use according to [47], wherein the antigen is an antigenic peptide, and the number of amino acid residues in the antigenic peptide is 23 to 60.

[61] Use according to [47], wherein the vaccine formulation is administered in combination with at least one type of antibody for use in cancer treatment, and the antibody is an antibody inhibiting an immunosuppressive signal from tumor, or at least one type of antibody activating a costimulatory signal of immunocytes.

[62] Use according to [47], wherein the vaccine formulation is administered in combination with at least one type of antibody for use in cancer treatment, and the antibody is an anti-CTLA4 antibody, an anti-PD1 antibody, an anti-PDL1 antibody, an anti-OX40 antibody, or an anti-4-1BB antibody.

[63] Use according to [47], wherein the vaccine formulation is administered in combination with at least one type of antibody for use in cancer treatment, and the antibody is an anti-PDL1 antibody.

### Advantageous Effects of Invention

Use of the vaccine formulation comprising a complex of a hyaluronic acid derivative having an introduced hydrophobic group, and an antigen according to the present invention improves the accumulation of the antigen to lymph node, enables marked induction of antigen-specific CTL, and enables potentiation of an anti-cancer effect by immunity. The complex and the vaccine formulation of the present invention also have characteristics excellent in safety, particularly, safety in long-term administration, because the hyaluronic acid derivative used is also excellent in safety.

### BRIEF DESCRIPTION OF DRAWINGS

FIG. 1 is a graph showing the ratio of the number of interferon gamma-producing CD8⁺ cells to the number of all CD8⁺ cells in BALB/c mouse-derived spleen cells (ordinate) when mERK2 p121 was used as a peptide for CD8⁺ T cell stimulation. Each sample is as follows: NT: spleen cells derived from an untreated mouse, CpG: spleen cells derived from a mouse given an antigenic peptide (mERK2 p121) and CpG oligo DNA, CHP+CpG: spleen cells derived from a mouse given a complex of CHP (CHP-80T; 80 k) and the antigenic peptide, and CpG oligo DNA, 99k41+CpG: spleen cells derived from a mouse given a complex of the antigenic peptide and a HA derivative (99k HA-C₆-Chol-41%), and CpG oligo DNA.

FIG. 2-1 is a graph showing the ratio of the number of interferon gamma-producing CD8⁺ cells to the number of all CD8⁺ cells in BALB/c mouse-derived spleen cells (ordinate) when mERK2 p121 was used as a peptide for CD8⁺ T cell stimulation. Each sample is as follows: NT: spleen cells derived from an untreated mouse, 99k41: spleen cells derived from a mouse given a complex of an antigenic peptide (mERK2 p121) and a HA derivative (99k HA-C₆-Chol-41%), 99k41+CpG: spleen cells derived from a mouse given a complex of the antigenic peptide and the HA derivative, and CpG oligo DNA, 9Yk41+polyIC: spleen cells derived from a mouse given a complex of the antigenic peptide and the HA derivative, and polyIC, 99k41+QuilA: spleen cells derived from a mouse given a complex of the antigenic peptide and the HA derivative, and QuilA, 99k41+sting: spleen cells derived from a mouse given a complex of the antigenic peptide and the HA derivative, and Sting.

FIG. 2-2 is a graph showing the ratio of the number of interferon gamma-producing CD4⁺ cells to the number of all CD4⁺ cells in BALB/c mouse-derived spleen cells (ordinate) when mERK2 p121 was used as a peptide for CD4⁺ T cell stimulation. Each sample is the same as in FIG. 2-1.

FIG. 3-1 is a graph showing the ratio of the number of interferon gamma-producing CD8⁺ cells to the number of all CD8⁺ cells in BALB/c mouse-derived spleen cells (ordinate) when mERK2 p121 was used as a peptide for CD8⁺ T cell stimulation. Each sample is as follows: NT: spleen cells derived from an untreated mouse, 99k41+CpG: spleen cells derived from a mouse given a complex of an antigenic peptide (mERK2 p121) and a HA derivative (99k HA-C₆-Chol-41%), and CpG oligo DNA, 99k41+R848: spleen cells derived from a mouse given a complex of the antigenic peptide and the HA derivative, and R848, 99k41+MPL: spleen cells derived from a mouse given a complex of the antigenic peptide and the HA derivative, and MPL.

FIG. 3-2 is a graph showing the ratio of the number of interferon gamma-producing CD4⁺ cells to the number of all CD4⁺ cells in BALB/c mouse-derived spleen cells (ordinate) when mERK2 p121 was used as a peptide for CD4⁺ T cell stimulation. Each sample is the same as in FIG. 3-1.

FIG. 4 is a graph showing the ratio of the number of interferon gamma-producing CD8⁺ cells to the number of all CD8⁺ cells in BALB/c mouse-derived spleen cells (ordinate) when MAGE-A4 p265 was used as a peptide for CD8⁺ T cell stimulation. Each sample is as follows: NT: spleen cells derived from an untreated mouse, peptide+CpG: spleen cells derived from a mouse given an antigenic peptide (MAGE-A4 p264) and CpG oligo DNA, CHP/Wt+CpG: spleen cells derived from a mouse given a complex of CHP (80 k) and the antigenic peptide, and CpG oligo DNA, 99k41/Wt+CpG: spleen cells derived from a mouse given a complex of the antigenic peptide and a HA derivative (99k HA-C₆-Chol-41%), and CpG oligo DNA.

FIG. 5 is a graph showing the ratio of the number of interferon gamma-producing CD8⁺ cells to the number of all CD8⁺ cells in BALB/c mouse-derived spleen cells (ordinate) when MAGE-A4 p265 was used as a peptide for CD8⁺ T cell stimulation. Each sample is as follows: NT: spleen cells derived from an untreated mouse, CHP/Wt: spleen cells derived from a mouse given a complex of CHP (80 k) and an antigenic peptide (MAGE-A4 p264), 99k41/Wt: spleen cells derived from a mouse given a complex of the antigenic peptide and a HA derivative (99k HA-C₆-Chol-41%).

FIG. 6 is a graph showing the ratio of the number of interferon gamma-producing CD8⁺ cells to the number of all CD8⁺ cells in BALB/c mouse-derived spleen cells (ordinate) when MAGE-A4 p265 was used as a peptide for CD8⁺ T cell stimulation. Each sample is as follows: NT: spleen cells derived from an untreated mouse, 99k41/Wt+CpG: spleen cells derived from a mouse given a complex of an antigenic peptide (MAGE-A4 p264) and a HA derivative (99k HA-C₆-Chol-41%), and CpG oligo DNA, 99k41/LLLL+CpG: spleen cells derived from a mouse given a complex of an antigenic peptide (MAGE-A4 p264-4L) and the HA derivative, and CpG oligo DNA, 99k41/WWWW+CpG: spleen cells derived from a mouse given a complex of an antigenic peptide (MAGE-A4 p264-4W) and the HA derivative, and CpG oligo DNA.

FIG. 7-1 is a graph showing the ratio of the number of interferon gamma-producing CD8⁺ cells to the number of all CD8⁺ cells in C57BL/6 mouse-derived spleen cells (ordinate) when TRP1 was used as a peptide for CD8⁺ T cell stimulation. Each sample is as follows: NT: spleen cells derived from an untreated mouse, CHP/6Y+CpG: spleen cells derived from a mouse given a complex of CHP and an antigenic peptide (TRP2TRP1gp100-6Y), and CpG oligo DNA, 99k43/6Y+CpG: spleen cells derived from a mouse given a complex of the antigenic peptide and a HA derivative (99k HA-C₆-Chol-43%), and CpG oligo DNA.

FIG. 7-2 is a graph showing the ratio of the number of interferon gamma-producing CD8⁺ cells to the number of all CD8⁺ cells in C57BL/6 mouse-derived spleen cells (ordinate) when TRP2 was used as a peptide for CD8⁺ T cell stimulation. Each sample is the same as in FIG. 7-1.

FIG. 7-3 is a graph showing the ratio of the number of interferon gamma-producing CD8⁺ cells to the number of all CD8⁺ cells in C57BL/6 mouse-derived spleen cells (ordinate) when gp100 was used as a peptide for CD8⁺ T cell stimulation. Each sample is the same as in FIG. 7-1.

FIG. 8-1 is a graph showing the ratio of the number of interferon gamma-producing CD8⁺ cells to the number of all CD8⁺ cells in C57BL/6 mouse-derived spleen cells (ordinate) when TRP1 was used as a peptide for CD8⁺ T cell stimulation. Each sample is as follows: NT: spleen cells derived from an untreated mouse, 99k43/6G+CpG: spleen cells derived from a mouse given a complex of an antigenic peptide (TRP2TRP1gp100-6G) and a HA derivative (99k HA-C₆-Chol-43%), and CpG oligo DNA, 99k43/6Y+CpG: spleen cells derived from a mouse given a complex of an antigenic peptide (TRP2TRP1gp100-6Y) and the HA derivative, and CpG oligo DNA, 99k43/4L+CpG: spleen cells derived from a mouse given a complex of an antigenic peptide (TRP2TRP1gp100-4L) and the HA derivative, and CpG oligo DNA, 99k43/6L+CpG: spleen cells derived from a mouse given a complex of an antigenic peptide (TRP2TRP1gp100-6L) and the HA derivative, and CpG oligo DNA.

FIG. 8-2 is a graph showing the ratio of the number of interferon gamma-producing CD8⁺ cells to the number of all CD8⁺ cells in C57BL/6 mouse-derived spleen cells (ordinate) when TRP2 was used as a peptide for CD8⁺ T cell stimulation. Each sample is the same as in FIG. 8-1.

FIG. 8-3 is a graph showing the ratio of the number of interferon gamma-producing CD8⁺ cells to the number of all CD8⁺ cells in C57BL/6 mouse-derived spleen cells (ordinate) when gp100 was used as a peptide for CD8⁺ T cell stimulation. Each sample is the same as in FIG. 8-1.

FIG. 9-1 is a graph showing the ratio of the number of interferon gamma-producing CD8⁺ cells to the number of all CD8⁺ cells in BALB/c mouse-derived spleen cells (ordinate) when AH1 was used as a peptide for CD8⁺ T cell stimulation. Each sample is as follows: NT: spleen cells derived from an untreated mouse, CHP/6Y+CpG: spleen cells derived from a mouse given a complex of CHP and an antigenic peptide (AH1gp70-6Y), and CpG oligo DNA, 99k43/6Y+CpG: spleen cells derived from a mouse given a complex of the antigenic peptide and a HA derivative (99k HA-C₆-Chol-43%), and CpG oligo DNA.

FIG. 9-2 is a graph showing the ratio of the number of interferon gamma-producing CD4⁺ cells to the number of all CD4⁺ cells in BALB/c mouse-derived spleen cells (ordinate) when gp70 was used as a peptide for CD4⁺ T cell stimulation. Each sample is the same as in FIG. 9-1.

FIG. 10-1 is a graph showing the ratio of the number of interferon gamma-producing CD8⁺ cells to the number of all CD8⁺ cells in BALB/c mouse-derived spleen cells (ordinate) when AH1 was used as a peptide for CD8⁺ T cell stimulation. Each sample is as follows: NT: spleen cells derived from an untreated mouse. CHP/6L+CpG: spleen cells derived from a mouse given a complex of CHP and an antigenic peptide (AH1gp70-6L), and CpG oligo DNA, 99k43/6L+CpG: spleen cells derived from a mouse given a complex of the antigenic peptide and a HA derivative (99k HA-C₆-Chol-43%), and CpG oligo DNA.

FIG. 10-2 is a graph showing the ratio of the number of interferon gamma-producing CD4⁺ cells to the number of all CD4⁺ cells in BALB/c mouse-derived spleen cells (ordinate) when gp70 was used as a peptide for CD4⁺ T cell stimulation. Each sample is the same as in FIG. 10-1.

FIG. 11-1 is a graph showing the ratio of the number of interferon gamma-producing CD8⁺ cells to the number of all CD8⁺ cells in BALB/c mouse-derived spleen cells (ordinate) when AH1 was used as a peptide for CD8⁺ T cell stimulation. Each sample is as follows: NT: spleen cells derived from an untreated mouse, 99k43/6G+CpG: spleen cells derived from a mouse given a complex of an antigenic peptide (AH1gp70-6G) and a HA derivative (99k HA-C₆-Chol-43%), and CpG oligo DNA, 99k43/6Y+CpG: spleen cells derived from a mouse given a complex of an antigenic peptide (AH1gp70-6Y) and the HA derivative, and CpG oligo DNA, 99k43/4L+CpG: spleen cells derived from a mouse given a complex of an antigenic peptide (AH1gp70-4L) and the HA derivative, and CpG oligo DNA, 99k43/6L+CpG: spleen cells derived from a mouse given a complex of an antigenic peptide (AH1gp70-6L) and the HA derivative, and CpG oligo DNA.

FIG. 11-2 is a graph showing the ratio of the number of interferon gamma-producing CD4⁺ cells to the number of all CD4⁺ cells in BALB/c mouse-derived spleen cells (ordinate) when gp70 was used as a peptide for CD4⁺ T cell stimulation. Each sample is the same as in FIG. 11-1.

FIG. 12-1 is a graph showing changes in the average tumor volume of a mouse fibrosarcoma CMS5a-bearing BALB/c mouse group (ordinate). Each group is as follows: control: a mouse group given no sample, peptide: a mouse group given an antigenic peptide (mERK2 p121) and CpG oligo DNA, emulsion: a mouse group given an emulsion formulation produced from the antigenic peptide, and CpG oligo DNA, CHP: a mouse group given a complex of CHP and the antigenic peptide, and CpG oligo DNA, 99k41: a mouse group given a complex of the antigenic peptide and a HA derivative (99k HA-C₆-Chol-41%), and CpG oligo DNA.

FIG. 12-2 is a graph showing changes in the tumor volume of each individual of a mouse fibrosarcoma CMS5a-bearing BALB/c mouse group (ordinate) on a group basis. Each group is the same as in FIG. 12-1.

FIG. 13-1 is a graph showing changes in the average tumor volume of a mouse fibrosarcoma CMS5a-bearing BALB/c mouse group (n=5) (ordinate). Each group is as follows: control: a mouse group given no sample, CpG: a mouse group given CpG oligo DNA, CHP: a mouse group given a complex of CHP and an antigenic peptide (mERK2 p121), and CpG oligo DNA, 99k41: a mouse group given a complex of the antigenic peptide and a HA derivative (99k HA-C₆-Chol-41%), and CpG oligo DNA.

FIG. 13-2 is a graph showing changes in the tumor volume of each individual of a mouse fibrosarcoma CMS5a-bearing BALB/c mouse group (n=5) (ordinate) on a group basis. Each group is the same as in FIG. 13-1.

FIG. 14-1 is a graph showing changes in the average tumor volume of a mouse fibrosarcoma CMS5a-bearing BALB/c mouse group (n=5) (ordinate). Each group is as follows: control: a mouse group given no sample, aCTLA4/aPD1/aPDL1: a mouse group given an anti-CTLA4 antibody, an anti-PD1 antibody, and an anti-PDL1 antibody, 99k41: a mouse group given a complex of an antigenic peptide (mERK2 p121) and a HA derivative (99k HA-C₆-Chol-41%), and CpG oligo DNA.

FIG. 14-2 is a graph showing changes in the tumor volume of each individual of a mouse fibrosarcoma CMS5a-bearing BALB/c mouse group (n=5) (ordinate) on a group basis. Each group is the same as in FIG. 14-1.

FIG. 15-1 is a graph showing changes in the average tumor volume of a mouse fibrosarcoma CMS5a-bearing BALB/c mouse group (n=5) (ordinate). Each group is as follows: control: a mouse group given no sample, aCTLA4/aPDL1/aOX40/a4-1BB: a mouse group given an anti-CTLA4 antibody, an anti-PDL1 antibody, an anti-OX40 antibody, and an anti-4-1 BB antibody, 99k41: a mouse group given a complex of an antigenic peptide (mERK2 p121) and a HA derivative (99k HA-C₆-Chol-41%), and CpG oligo DNA, 99k41+aCTLA4/aPDL1/aOX40/a4-1BB: a mouse group given a complex of the antigenic peptide and the HA derivative, CpG oligo DNA, and an anti-CTLA4 antibody, an anti-PDL1 antibody, an anti-OX40 antibody, and an anti-4-1BB antibody.

FIG. 15-2 is a graph showing changes in the tumor volume of each individual of a mouse fibrosarcoma CMS5a-bearing BALB/c mouse group (n=5) (ordinate) on a group basis. Each group is the same as in FIG. 15-1.

FIG. 16-1 is a graph showing changes in the average tumor volume of a mouse colorectal cancer cell CT26-bearing BALB/c mouse group (ordinate). Each group is as follows: control: a mouse group given no sample, emulsion: a mouse group given an emulsion formulation produced from an antigenic peptide (AH1gp70-6L), and CpG oligo DNA, CHP: a mouse group given a complex of CHP and the antigenic peptide, and CpG oligo DNA, 99k43: a mouse group given a complex of the antigenic peptide and a HA derivative (99k HA-C₆-Chol-43%), and CpG oligo DNA.

FIG. 16-2 is a graph showing changes in the tumor volume of each individual of a mouse colorectal cancer cell CT26-bearing BALB/c mouse group (n=5) (ordinate) on a group basis. Each group is the same as in FIG. 16-1.

FIG. 17-1 is a graph showing changes in the average tumor volume of a mouse melanoma B16F10-bearing C57BL/6 mouse group (ordinate). Each group is as follows: control: a mouse group given no sample, CHP: a mouse group given a complex of CHP and an antigenic peptide (TRP2TRP1gp100-6Y), and CpG oligo DNA, 99k43: a mouse group given a complex of the antigenic peptide and a HA derivative (99k HA-C₆-Chol-43%), and CpG oligo DNA.

FIG. 17-2 is a graph showing changes in the tumor volume of each individual of a mouse melanoma B16F10-bearing C57BL/6 mouse group (n=5) (ordinate) on a group basis. Each group is the same as in FIG. 17-1.

FIG. 18-1 is a graph showing fluorescence intensity per cell of BALB/c mouse lymph node 24 hours after sample administration in a lymph node migration test. Each administration sample is as follows: NT: no sample added, LPA: an antigenic peptide (fluorescently labeled mERK2 p121 (fluorescein-conjugated)), CHP: a complex of CHP and the antigenic peptide, 10k17: a complex of the antigenic peptide and a HA derivative (10k HA-C₆-Chol-17%), 10k25: a complex of the antigenic peptide and a HA derivative (10k HA-C₆-Chol-25%), 10k42: a complex of the antigenic peptide and a HA derivative (10k HA-C-Chol-42%), 50k23: a complex of the antigenic peptide and a HA derivative (50k HA-C₆-Chol-23%), 50k43: a complex of the antigenic peptide and a HA derivative (50k HA-C₆-Chol-43%), 99k24: a complex of the antigenic peptide and a HA derivative (99k HA-C₆-Chol-24%), 99k43: a complex of the antigenic peptide and a HA derivative (99k HA-C₆-Chol-43%).

FIG. 18-2 is a graph showing fluorescence intensity per cell of BALB/c mouse lymph node 72 hours after sample administration in a lymph node migration test. Each administration sample is the same as in FIG. 18-1.

FIG. 19 is a graph showing fluorescence intensity per cell of BALB/c mouse lymph node 24 hours after sample administration in a lymph node migration test. Each administration sample is as follows: NT: no sample added, peptide: an antigenic peptide (fluorescently labeled mERK2 p121 (fluorescein-conjugated)), 99k41: a complex of the antigenic peptide and a HA derivative (99k HA-C₆-Chol-41%), 10kHA-Ala-Chol30: a complex of the antigenic peptide and a HA derivative (10k HA-Ala-C₆-Chol-30%), 10kHA-Ser-Chol28: a complex of the antigenic peptide and a HA derivative (10k HA-Ser-C₆-Chol-28%), 10kHA-Gly-Chol32: a complex of the antigenic peptide and a HA derivative (10k HA-Gly-C₆-Chol-32%), 10kHA-Thr-Chol32: a complex of the antigenic peptide and a HA derivative (10k HA-Thr-C₆-Chol-32%), 10kHA-Asn-Chol20: a complex of the antigenic peptide and a HA derivative (10k HA-Asn-C₆-Chol-20%), 10kHA-Asp-Chol32: a complex of the antigenic peptide and a HA derivative (10k HA-Asp-C₆-Chol-32%), 10kHA-Phe-Chol31: a complex of the antigenic peptide and a HA derivative (10k HA-Phe-C₆-Chol-31%), 10kHA-Tyr-Chol32: a complex of the antigenic peptide and a HA derivative (10k HA-Tyr-C₆-Chol-32%), 10kHA-Ile-Chol28: a complex of the antigenic peptide and a HA derivative (10k HA-Ile-C₆-Chol-28%), 10kHA-Leu-Chol31: a complex of the antigenic peptide and a HA derivative (10k HA-Leu-C₆-Chol-31%), 10kHA-Val-Chol32: a complex of the antigenic peptide and a HA derivative (10k HA-Val-C₆-Chol-32%), 10kHA-Trp-Chol24: a complex of the antigenic peptide and a HA derivative (10k HA-Trp-C₆-Chol-24%), 10kHA-Gln-Chol32: a complex of the antigenic peptide and a HA derivative (10k HA-Gln-C₆-Chol-32%), 10kHA-Glu-Chol32: a complex of the antigenic peptide and a HA derivative (10k HA-Glu-C₆-Chol-32%).

FIG. 20-1 is a graph showing changes in the average tumor volume of a mouse melanoma B16F10-bearing C57BL/6 mouse group (ordinate). Each group is as follows: control: a mouse group given no sample, 99k42: a mouse group given a complex of an antigenic peptide and a HA derivative (99k HA-C₆-Chol-42%), and CpG oligo DNA, 50k42: a mouse group given a complex of the antigenic peptide and a HA derivative (50k HA-C₆-Chol-42%), and CpG oligo DNA, 10k43: a mouse group given a complex of the antigenic peptide and a HA derivative (10k HA-C₆-Chol-43%), and CpG oligo DNA.

FIG. 20-2 is a graph showing changes in the tumor volume of each individual of a mouse melanoma B16F10-bearing C57BL/6 mouse group (n=5) (ordinate) on a group basis. Each group is the same as in FIG. 20-1.

FIG. 21-1 is a graph showing changes in the average tumor volume of a mouse melanoma B16F10-bearing C57BL/16 mouse group (ordinate). Each group is as follows: control: a mouse group given no sample, 10kHA-Ala-Chol30: a mouse group given a complex of an antigenic peptide and an amino acid-modified HA derivative (10k HA-Ala-C₆-Chol-30%), and CpG oligo DNA, 10kHA-Gln-Chol32: a mouse group given a complex of the antigenic peptide and an amino acid-modified HA derivative (10k HA-Gln-C₆-Chol-32%), and CpG oligo DNA.

FIG. 21-2 is a graph showing changes in the tumor volume of each individual of a mouse melanoma B16F10-bearing C57BL/6 mouse group (n=5) (ordinate) on a group basis. Each group is the same as in FIG. 21-1.

FIG. 22-1 is a graph showing changes in the average tumor volume of a mouse melanoma B16F10-bearing C57BL/6 mouse group (ordinate). Each group is as follows: control: a mouse group given no sample, 99k42+PolyIC: a mouse group given a complex of an antigenic peptide and a HA derivative (99k HA-C₆-Chol-42%), and polyIC.

FIG. 22-2 is a graph showing changes in the tumor volume of each individual of a mouse melanoma B16F10-bearing C57BL/6 mouse group (n=5) (ordinate) on a group basis. Each group is the same as in FIG. 22-1.

FIG. 23-1 is a graph showing changes in the average tumor volume of a mouse melanoma B16F10-bearing C57BL/6 mouse group (ordinate). Each group is as follows: control: a mouse group given no sample, 99k42+Sting: a mouse group given a complex of an antigenic peptide and a HA derivative (99k HA-C₆-Chol-42%), and a Sting agonist, 99k42+R848: a mouse group given a complex of the antigenic peptide and a HA derivative (99k HA-C₆-Chol-42%), and R848.

FIG. 23-2 is a graph showing changes in the tumor volume of each individual of a mouse melanoma B16F10-bearing C57BL/6 mouse group (n=5) (ordinate) on a group basis. Each group is the same as in FIG. 23-1.

FIG. 24-1 is a graph showing the ratio of the number of TRP2-specific CD8⁺ cells to the number of all CD8⁺ cells in mouse melanoma B16F10-bearing mouse-derived tumor or lymph node (ordinate) when H-2K TRP-2 Tetramer-SVYDFFVWL-APC was used as a tetramer. Each sample is as follows: NT: cells derived from an untreated cancer-bearing mouse, 99k41: cells derived from a cancer-bearing mouse given a complex of an antigenic peptide (TRP2TRP1gp100-6Y) and a HA derivative (99k HA-C₆-Chol-41%), and CpG oligo DNA.

FIG. 24-2 is a graph showing the ratio of the number of gp100-specific CD8⁺ cells to the number of all CD8⁺ cells in mouse melanoma B16F10-bearing mouse-derived tumor or lymph node (ordinate) when H-2D^{b} gp100 Tetramer-EGSRNQDWL-PE was used as a tetramer. Each sample is as follows: NT: cells derived from an untreated cancer-bearing mouse, 99k41: cells derived from a cancer-bearing mouse given a complex of an antigenic peptide (TRP2TRP1gp100-6Y) and a HA derivative (99k HA-C₆-Chol-41%), and CpG oligo DNA.

### DESCRIPTION OF EMBODIMENTS

Hereinafter, the present invention will be described further specifically.

The complex of a hyaluronic acid derivative having an introduced hydrophobic group, and an antigen according to the present invention is a complex of a hyaluronic acid derivative comprising at least one disaccharide unit (which is also a repeating unit) represented by the formula (I), or a hyaluronic acid derivative comprising at least one disaccharide unit (which is also a repeating unit) represented by the formula (II), and an antigen. The complex can be used to produce the vaccine formulation of the present invention. The present specification also includes the disclosure of a method for producing the complex of a hyaluronic acid derivative having an introduced hydrophobic group, and an antigen according to the present invention.

### Definition

The term “steryl group” described herein is not particularly limited as long as the group has a steroid skeleton. In this context, examples of the steroid specifically include cholesterol, dehydrocholesterol, coprostanol, coprosterol, cholestanol, campestanol, ergostanol, stigmastanol, coprostanol, stigmasterol, sitosterol, lanosterol, ergosterol, simiarenol, bile acid (cholanic acid, lithocholic acid, hyodeoxycholic acid, chenodeoxycholic acid, ursodeoxycholic acid, deoxycholic acid, apocholic acid, cholic acid, dehydrocholic acid, glycocholic acid, and taurocholic acid), testosterone, estradiol, progesterone, cortisol, cortisone, aldosterone, corticosterone, and deoxycorticosterone. Examples of the steryl group include a cholesteryl group, a stigmasteryl group, a lanosteryl group, an ergosteryl group, a cholanoyl group, and a choloyl group and preferably include a cholesteryl group (particularly, a cholest-5-en-3β-yl group represented by the following formula) and a cholanoyl group (particularly, a 5β-cholan-24-oyl group represented by the following formula).

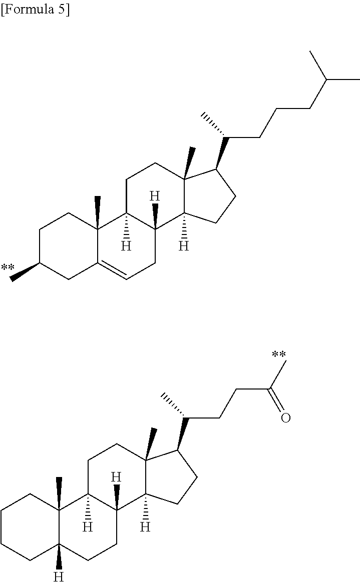

In this context, two asterisks represent a binding position.

The term “C₁₋₂₀ alkyl” described herein means a linear or branched alkyl group having 1 to 20 carbon atoms. Examples thereof include “C₁₋₄ alkyl” such as methyl, ethyl, n-propyl, i-propyl, n-butyl, s-butyl, i-butyl, and t-butyl and further include n-pentyl, 3-methylbutyl, 2-methylbutyl, 1-methylbutyl, 1-ethylpropyl, n-hexyl, 4-methylpentyl, 3-methylpentyl, 2-methylpentyl, 1-methylpentyl, 3-ethylbutyl, and 2-ethylbutyl. The C₁₋₂₀ alkyl also includes “C₁₋₁₂ alkyl” having 1 to 12 carbon atoms and “C₁₋₆ alkyl” having 1 to 6 carbon atoms.

The term “C₁₋₆ alkyl” described herein means a linear or branched alkyl group having 1 to 6 carbon atoms. Examples thereof include “C₁₋₄ alkyl” such as methyl, ethyl, n-propyl, i-propyl, n-butyl, s-butyl, i-butyl, and t-butyl.

The term “C₁₋₆ alkylcarbonyl” described herein means an alkylcarbonyl group whose alkyl moiety is already mentioned C₁₋₆ alkyl. Examples thereof include “C₁₋₄ alkylcarbonyl” such as acetyl, propionyl, n-propylcarbonyl, i-propylcarbonyl, n-butylcarbonyl, s-butylcarbonyl, i-butylcarbonyl, and t-butylcarbonyl.

The term “C₁₋₆ alkoxy” described herein means an alkyloxy group whose alkyl moiety is already mentioned C₁₋₆ alkyl. Examples thereof include “C₁₋₄ alkoxy” such as methoxy (H₃C—O—), ethoxy, n-propoxy, i-propoxy, n-butoxy, s-butoxy, i-butoxy, and t-butoxy.

The term “C₁₋₆ alkylthio” described herein means an alkylthio group whose alkyl moiety is already mentioned C₁₋₆ alkyl. Examples thereof include methylthio (H₃C—S—), ethylthio, n-propylthio, i-propylthio, n-butylthio, s-butylthio, i-butylthio, and t-butylthio and preferably include methylthio.

The term “amino-C₂₋₂₀ alkyl” described herein means a linear or branched alkyl group having 2 to 20 carbon atoms and having an amino group as a substituent. The amino group may be positioned, for example, on a terminal carbon atom of the alkyl group. The amino-C₂₋₂₀ alkyl also includes “amino-C₂₋₁₂ alkyl” having 2 to 12 carbon atoms.

The term “hydroxy-C₂₋₂₀ alkyl” described herein means a linear or branched alkyl group having 2 to 20 carbon atoms and having a hydroxy group as a substituent. The hydroxy group may be positioned, for example, on a terminal carbon atom of the alkyl group. The hydroxy-C₂₋₂₀ alkyl also includes “hydroxy-C₂₋₁₂ alkyl” having 2 to 12 carbon atoms.

The term “C₂₋₃₀ alkylene” described herein means a linear or branched divalent saturated hydrocarbon group having 2 to 30 carbon atoms. Examples thereof include ethylene and propylene and include C₂₋₂₀ alkylene, C₂₋₈ alkylene, and a group-(CH₂)ₙ— (wherein n is 2 to 30, preferably 2 to 20, more preferably 2 to 15).

The term “C₁₋₅ alkylene” described herein means a linear or branched divalent saturated hydrocarbon group having 1 to 5 carbon atoms. Examples thereof include methylene, ethylene (ethane-1,2-diyl and ethane-1,1-diyl), propylene (propane-1,1-diyl and propane-1,2-diyl), butane-1,4-diyl, and pentane-1,5-diyl.

The term “C₂₋₁₀ alkylene” described herein means a linear or branched divalent saturated hydrocarbon group having 2 to 10 carbon atoms. Examples thereof include ethylene (ethane-1,2-diyl and ethane-1,1-diyl), propylene (propane-1,1-diyl, propane-1,2-diyl, and propane-1,3-diyl), butane-1,4-diyl, pentane-1,5-diyl, hexane-1,6-diyl, heptane-1,7-diyl, and octane-1,8-diyl. The “C₂₋₁₀ alkylene” includes “C₂₋₈ alkylene” having 2 to 8 carbon atoms and “C₂₋₆ alkylene” having 2 to 6 carbon atoms.

The term “C₂₋₈ alkylene” described herein means a linear or branched divalent saturated hydrocarbon group having 2 to 8 carbon atoms. Examples thereof include ethylene (ethane-1,2-diyl and ethane-1,1-diyl), propylene (propane-1,1-diyl, propane-1,2-diyl, and propane-1,3-diyl), butane-1,4-diyl, pentane-1,5-diyl, hexane-1,6-diyl, heptane-1,7-diyl, and octane-1,8-diyl.

The term “C₂₋₈ alkenylene” described herein means a linear or branched divalent saturated hydrocarbon group having 2 to 8 carbon atoms and containing one or more double bonds. Examples thereof include —CH═CH—, —C(CH₃)═CH—, 2-butene-1,4-diyl, hepta-2,4-diene-1,6-diyl and octa-2,4,6-triene-1,8-diyl. If geometric isomerism is present, respective isomers and their mixtures are also included therein.

In the present invention, the “aryl” means an aromatic carbocyclic group, for example, an aromatic carbocyclic group having 6 to 14 carbon atoms. Examples of the aryl include phenyl and naphthyl (1-naphthyl and 2-naphthyl). Examples of the aryl substituted by one or more hydroxy groups include 4-hydroxyphenyl.

In the present invention, the “heteroaryl” means an aromatic cyclic group containing one or more heteroatoms selected from a nitrogen atom, an oxygen atom and a sulfur atom as ring-constituting atoms, and may be partially saturated. The ring may be monocyclic, or bicyclic heteroaryl condensed with a benzene ring or a monocyclic heteroaryl ring. The number of ring-constituting atoms is, for example, 4 to 15, preferably 5 to 14, more preferably 6 to 10. Examples of the heteroaryl include furyl, thienyl, pyrrolyl, imidazolyl, pyrazolyl, thiazolyl, isothiazolyl, oxazolyl, isoxazolyl, oxadiazolyl, thiadiazolyl, triazolyl, tetrazolyl, pyridyl, pyrimidyl, pyridazinyl, pyrazinyl, triazinyl, benzofuranyl, benzothienyl, benzothiadiazolyl, benzothiazolyl, benzoxazolyl, benzoxadiazolyl, benzimidazolyl, indolyl, isoindolyl, indazolyl, quinolyl, isoquinolyl, cinnolinyl, quinazolinyl, quinoxalinyl, benzodioxolyl, indolizinyl, imidazopyridyl and preferably indol-2-yl.

Examples of the “divalent C₂₋₅₀ hydrocarbon group” described herein include, but are not particularly limited to, linear, branched, cyclic and partially cyclic alkylene groups, alkenylene groups and alkynylene groups having 2 to 50 carbon atoms. Each of these groups may be a divalent aromatic ring or may contain an aromatic ring as a portion of the structure.

The “divalent C₂₋₅₀ polyalkyleneoxy” described herein is not particularly limited, and an alkylene group in a repeating unit may be linear or branched. Examples of the “divalent C₂₋₅₀ polyalkyleneoxy” include a divalent C₂₋₅₀ polyethyleneoxy group, a C₃₋₄₈ polypropyleneoxy group, and a C₃₋₄₈ polybutyleneoxy group. Each of these groups may be linked to another group via an oxygen atom or a carbon atom. Examples of the C₂₋₅₀ polyethyleneoxy group include —O(CH₂CH₂O)₁₋₂₅—, —(CH₂CH₂O)₁₋₂₅—, —(OCH₂CH₂)₁₋₂₅—, and —(CH₂CH₂O)₁₋₂₄—(CH₂CH₂)—.

The term “salt substance” described herein is not particularly limited as long as the salt substance is inorganic matter soluble in water. Examples thereof include: calcium salts such as calcium chloride and calcium phosphate; magnesium salts such as magnesium sulfate and magnesium chloride; aluminum salts such as aluminum sulfate and aluminum chloride; potassium salts such as potassium sulfate, potassium carbonate, potassium nitrate, potassium chloride, potassium bromide, and potassium iodide; sodium salts such as sodium bicarbonate, sodium carbonate, sodium sulfate, sodium nitrate, sodium chloride, sodium bromide, sodium iodide, sodium silicate, trisodium phosphate, disodium phosphate, sodium borate, sodium acetate, and sodium citrate; and lithium salts such as lithium chloride, lithium bromide, lithium iodide, and lithium carbonate, and preferably include sodium chloride, trisodium phosphate, disodium phosphate, potassium chloride, calcium chloride, and magnesium chloride.

The term “antigen” described herein refers to a substance that initiates immunity. The antigen is, for example, a substance that can induce the activation of lymphocytes such as T cells or B cells through presentation by antigen-presenting cells in lymph node or the spleen. The term “antigenic protein” refers to an antigen which is a protein. The “antigenic peptide” refers to an antigen which is a peptide. For example, the antigenic peptide is a portion of an amino acid sequence contained in the antigenic protein and comprises a T cell recognition epitope. A plurality of T cell recognition epitopes may be combined.

The term “adjuvant” described herein refers to a substance that potentiates immune reaction in a subject by administration to the subject in combination with a vaccine containing an antigen.

Hyaluronic Acid Derivative Comprising Repeating Unit Represented by Formula (I)

In one aspect, the hyaluronic acid derivative comprising a repeating unit represented by the formula (I) is composed substantially of (1) a repeating unit of the formula (I); or (2) repeating units of the formula (I) and the formula (IIIc).

When the hyaluronic acid derivative comprising a repeating unit represented by the formula (I) comprises two or more repeating units of the formula (I), the repeating units may be the same or different. The hyaluronic acid derivative may be modified at a position other than the repeating unit of the formula (I). For example, a hydroxy group may be converted to —O(C₁₋₆ alkyl), —O(formyl) or —O(C₁₋₆ alkylcarbonyl), etc., and a carboxyl group may be converted to an amide group or an ester group or may form a salt.

According to one aspect, the group —Z—N(R^{a})Y—X¹ in the formula (I) is selected from groups represented by the following formulas:

—NH—(CH₂)_{mz}—NH—R;

—NH—(CH₂)_{mz}—NH—COO—R;

—NH—(CH₂CH₂O)ₘ—CH₂CH₂—NH—COO—R;

—NH—(CH₂)_{mz}—COO—R;

—NH—(CH₂CH₂O)ₘ—CH₂CH₂—COO—R;

—NH—(CH₂)_{mz}—O—COO—R;

—NH—(CH₂CH₂O)ₘ—CH₂CH₂—O—COO—R;

—NH—(CH₂)_{mz}—S—R;

—NH—(CH₂CH₂O)ₘ—CH₂CH₂—S—R;

—NH—(CH₂)_{mz}—O—CO—CH(R¹⁰)—CH₂—S—R;

—NH—(CH₂)_{mz}—NHCO—CH(R¹⁰)—CH₂—S—R;

—NH—(CH₂CH₂O)ₘ—CH₂CH₂—NHCO—CH(R¹⁰)—CH₂—S—R;

—NH—(CH₂CH₂O)ₘ—CH₂CH₂—O—CO—CH(R¹⁰)—CH₂—S—R;

—NH—(CH₂)_{mz}—S—S—R; and

—Z—NR^{a}—Y—NR^{b}—COO—R

wherein mz is an integer of 2 to 30, R¹⁰ is a hydrogen atom or a methyl group, and R and m are as already defined herein.

The group is preferably a group selected from

—NH—(CH₂)_{mz}—NH—COO—R;

—NH—(CH₂CH₂O)ₘ—CH₂CH₂—NH—COO—R and

—NH—(CH₂)_{mz}—S—S—R

wherein mz, R, and m are as already defined herein.

In a preferred aspect, Z in the formula (I) is a direct bond. In one aspect, when Z in the formula (I) is a peptide linker, X¹ is —NR^{b}—COO—R.

Specific examples of Y in the formula (I) include —CH₂CH₂O—CH₂CH₂—S—S—CH₂CH₂O—CH₂CH₂—, —(CH₂CH₂O)₂—CH₂CH₂—S—S—CH₂CH₂O—CH₂CH₂—, —CH₂CH₂O—CH₂CH₂—S—S—(CH₂CH₂O)₂—CH₂CH₂— and —(CH₂CH₂O)₂—CH₂CH₂—S—S—(CH₂CH₂O)₂—CH₂CH₂—.

Y^{a} in the formula (I) is preferably —CH₂— and —CH₂—CH₂—.

Y^{b} in the formula (I) is preferably —CH₂—CH₂—, —CH(CH₃)CH₂—, 2-butene-1,4-diyl, hepta-2,4-diene-1,6-diyl or octa-2,4,6-triene-1,8-diyl, more preferably —CH₂—CH₂— or —CH(CH₃)CH₂—.

In one aspect, Z in the formula (I) is a peptide linker represented by —NH—[CH(—Z^{a})—CONH]ₙ₋₁—CH(—Z^{a})—CO—, wherein n is an integer of 2 to 30, and each Z^{a} independently represents a substituent in an α-amino acid represented by H₂N—CH(—Z^{a})—COOH. The peptide linker binds at its N terminus to a carboxyl group of the glucuronic acid moiety and binds at its C terminus to the group —N(—R^{a})—Y—X¹. Examples of the amino acids that can be used as amino acid residues of the peptide linker include α-amino acids, for example, natural (L) amino acids such as alanine, arginine, asparagine (Asn), aspartic acid, cysteine, glutamine, glutamic acid, glycine (Gly), histidine, isoleucine, leucine (Leu), lysine, methionine, phenylalanine (Phe), proline, serine, threonine, tryptophan, tyrosine, and valine, and their D forms. Every α-amino acid including synthesized amino acids can be used. Specifically, examples of Z^{a} include —CH₃, H₂NC(NH)NH(CH₂)₃—, and H₂NCOCH₂—. n Z may be the same or different. n is an integer of 2 to 30, preferably 2 to 10, more preferably 2 to 4. Preferred examples of the peptide linker include -Gly-Phe-Leu-Gly-, -Asn-Phe-Phe-, -Phe-Phe-, and -Phe-Gly-.

Specific examples of the group —Z—N(R^{a})Y—X¹ in the formula (I) include —NH—(CH₂)₂—NH—CO-cholesteryl, —NH—(CH₂)₄—NH—(CH₂)₃—NH—(CH₂)₃—NH—COO-cholesteryl, —NH—(CH₂)—NH—(CH₂)₄—NH—(CH₂)₃—NH—COO-cholesteryl, —NH—(CH₂)₄—NH—(CH₂)₃—NH—COO-cholesteryl, —NH—(CH₂)₄—N(—(CH₂)₃—NH₂)—COO-cholesteryl, —NH—(CH₂)₃—NH—(CH₂)₄—N(—(CH₂)₃—NH₂)—COO-cholesteryl, —NH—(CH₂)₃—NH—(CH₂)₄—N(—(CH₂)₃—NH—(CH₂)₃—NH₂)—COO-cholesteryl, —NH—(CH₂)₃—NH—(CH₂)₄—N(—(CH₂)₃—NH₂)—CO—NH-cholesteryl, —NH—(CH₂)₃—NH—(CH₂)₄—N(—(CH₂)₃—NH₂)—CO-cholesteryl, and —NH—(CH₂)₃—NH—(CH₂)₄—N(—(CH₂)₃—NH₂)-cholesteryl. In a preferred aspect of the group —Z—N(R^{a})Y—X¹, each of R^{a}, R^{b} and R^{c} is a hydrogen atom, Y is linear C₂₋₃₀ alkylene or —(CH₂CH₂O)ₘ—CH₂CH₂—, and Y^{a} is linear C₁₋₅ alkylene, or Y^{b} is linear C₂₋₈ alkylene or linear C₂₋₈ alkenylene.

In one aspect, in the group Z—N(R^{a})Y—X¹, Z is a direct bond, R^{a} is a hydrogen atom, Y is, for example, C₂₋₁₂ alkylene, preferably C₂₋₆ alkylene, more preferably C₆ alkylene, X¹ is —NR^{b}—COO—R, R^{b} is a hydrogen atom, and R is a cholesteryl group.

In one aspect, the hyaluronic acid derivative comprising a repeating unit represented by the formula (I) further comprises a repeating unit represented by the formula (IIIc). When two or more repeating units represented by the formula (IIIc) are contained, the repeating units may be the same or different.

Q⁺ in the formula (IIIc) is not particularly limited as long as Q⁺ is a counter cation that forms a salt with a carboxyl group in water. Divalent or higher valent Q⁺ forms a salt with a plurality of carboxyl groups according to the valence. Examples of the counter cation include: metal ions such as lithium ions, sodium ions, rubidium ions, cesium ions, magnesium ions, and calcium ions; and ammonium ions represented by the formula: N⁺R^{j}R^{k}R^{l}R^{m} (wherein R^{j}, R^{k}, R^{l} and R^{m} are each independently selected from a hydrogen atom and C₁₋₆ alkyl), and preferably include sodium ions, potassium ions, and tetraalkylammonium ions (e.g., tetra-n-butylammonium ions). R^{j}, R^{k}, R^{l} and R^{m} are preferably the same groups selected from C₁₋₆ alkyl, and are preferably n-butyl groups.

All of the groups R¹, R², R³, and R⁴ in the formula (I), and R^{1a}, R^{2a}, R^{3a} and R^{4a} in the formula (IIIc) are preferably hydrogen atoms. Both R^{a} and R^{b} are preferably hydrogen atoms.

The group R⁵ in the formula (I) is preferably acetyl.

In one aspect, the hyaluronic acid derivative comprising a repeating unit represented by the formula (I) is composed substantially of repeating units of the formulas (I) and (IIIc). In the hyaluronic acid derivative, for example, 80% or more, preferably 90% or more, more preferably 95% or more, of disaccharide repeating units consisting of D-glucuronic acid and N-acetylglucosamine contained in the derivative are repeating units of the formula (I) or (IIIc). In one aspect, the hyaluronic acid derivative is composed only of repeating units represented by the formula (I) and the formula (IIIc).

Y defined in the formula (I) may be, for example, —(CH₂)ₙₐ— (wherein na is selected from integers of 2 to 20, preferably 2 to 15, more preferably 2 to 12) and is preferably —(CH₂)₂—, —(CH₂)₆—, —(CH₂)₈— or —(CH₂)₁₂—, more preferably —(CH₂)₆—. Such Y is preferred from the viewpoint of precipitation formation and stable dispersion mentioned later.

According to one aspect, in the hyaluronic acid derivative comprising a repeating unit represented by the formula (I), the rate of introduction of the hydrophobic group to disaccharide repeating units present in the derivative is, for example, 1 to 50%, preferably 7 to 50%, more preferably 17 to 50%, still more preferably 20 to 45%. When the rate of introduction falls within the range described above, the hyaluronic acid derivative comprising a repeating unit represented by the formula (I) can efficiently form a complex with the antigen in a solution.

In this context, the rate of introduction of the hydrophobic group is calculated according to the following expression:

$$( Rate of introduction of hydrophobic group ) = \frac{\begin{matrix}( The number of disaccharide repeating units \\ having an introduced hydrophobic group )\end{matrix}}{\begin{matrix}( The number of disaccharide \\ repeating units present )\end{matrix} } \times 100 \qquad [ Expression 1 ]$$

In this context, the “disaccharide repeating units present in the derivative” include a repeating unit of the formula (I) wherein a hydrophobic group is introduced by the conversion of a carboxyl group to an amide group, and a repeating unit of the formula (IIIc) wherein no hydrophobic group is introduced. The rate of introduction can be controlled by reaction conditions, for example, the ratio of a reagent, and can be determined by, for example, NMR measurement.

The hyaluronic acid derivative comprising a repeating unit represented by the formula (I) is preferably synthesized by using hyaluronic acid composed only of a repeating unit represented by the formula (IIIc) or a derivative thereof as a starting material, wherein when all of R^{1c}, R^{2c}, R^{3c}, and R^{4c} are hydrogen atoms, R^{5c} is acetyl, and X^{c} is —O⁻Na⁺, a weight-average molecular weight is, for example, 1 kDa to 500 kDa, preferably 3 kDa to 500 kDa, more preferably 5 kDa to 200 kDa. In one aspect, the weight-average molecular weight of the starting material is preferably 1 kDa to 1000 kDa, more preferably 3 kDa to 200 kDa, from the viewpoint of complex formation with the antigen. The weight-average molecular weight of the starting material is preferably 1 kDa to 1000 kDa, more preferably 3 kDa to 500 kDa, still more preferably 5 kDa to 200 kDa, even more preferably 5 kDa to 150 kDa, from the viewpoint of the transfer of the complex to lymph node.

In general, hyaluronic acid and a derivative thereof are difficult to obtain as single products. Therefore, their molecular weights are calculated as number-average molecular weights or weight-average molecular weights. In the present invention, a weight-average molecular weight is calculated. Various methods known in the art, for example, a light scattering method, an osmotic pressure method, and a viscosity method, described in Seiichi Nakahama, et al, “Essential Polymer Science” (published by Kodansha Ltd., ISBN 4-06-153310-X) can be used as methods for measuring the weight-average molecular weight. A viscosity-average molecular weight described herein can also be measured by a method usually used in the technical field to which the present invention belongs, such as use of a Ubbelohde viscometer. In the case of using a commercially available hyaluronic acid and derivative thereof having a clearly specified molecular weight, the clearly specific numeric value may be used as a molecular weight.

In the hyaluronic acid derivative comprising a repeating unit of the formula (I), the hydrophobic group is introduced by the conversion of a carboxyl group of glucuronic acid as one of the saccharides of the disaccharide constituting the repeating unit to an amide group. The in vivo kinetics of a formulation to be produced using the hyaluronic acid derivative may be controlled by adjusting the degree of modification of the hyaluronic acid derivative.

When the rate of carboxyl group modification of the glucuronic acid moiety of the hyaluronic acid derivative comprising a repeating unit of the formula (I) is high, binding to a hyaluronic acid receptor including CD44 is suppressed so that the hyaluronic acid derivative serves as a drug carrier (including a vaccine) that remains long in the body. Furthermore, each organ including lymph node, and cells can be targeted by introducing a targeting element to the hyaluronic acid derivative. Examples of the targeting element include target tissue-specific peptides, antibodies, antibody fragments, aptamers, RGD peptides against cancer cells, folic acid, anisamide, transferrin, galactose for the liver, and tocopherol.

The rate of carboxyl group modification with the hydrophobic group, i.e., the rate of introduction of the hydrophobic group, of the glucuronic acid moiety of the hyaluronic acid derivative comprising a repeating unit of the formula (I) is preferably 4 to 60%, more preferably 5 to 50%, still more preferably 7 to 50%, even more preferably 7 to 45%, from the viewpoint of complex formation with the antigen. The rate is preferably 6 to 60%, more preferably 17 to 50%, still more preferably 20 to 50%, even more preferably 20 to 45%, from the viewpoint of the transfer of the complex to lymph node.

The combination of the molecular weight of the starting material and the rate of introduction of the hydrophobic group for the hyaluronic acid derivative comprising a repeating unit represented by the formula (I) is preferably 3 kDa to 500 kDa and 4 to 60%, more preferably 3 kDa to 200 kDa and 6 to 50%, still more preferably 5 kDa to 200 kDa and 6 to 50%, even more preferably 5 kDa to 150 kDa and 6 to 45%, from the viewpoint of complex formation with the antigen. The combination is preferably 3 kDa to 500 kDa and 4 to 60%, more preferably 3 kDa to 200 kDa and 6 to 50%, still more preferably 5 kDa to 200 kDa and 6 to 50%, even more preferably 5 kDa to 150 kDa and 20 to 45%, from the viewpoint of the transfer of the complex to lymph node. The combination is preferably 3 kDa to 200 kDa and 4 to 60%, more preferably 3 kDa to 200 kDa and 6 to 50%, still more preferably 5 kDa to 200 kDa and 6 to 50%, even more preferably 5 kDa to 150 kDa and 17 to 45%, from the viewpoint of stable dispersion. The combination is preferably 5 kDa to 27 kDa and 2 to 50%, more preferably 5 kDa to 27 kDa and 8 to 35%, still more preferably 5 kDa to 18 kDa and 8 to 35%, even more preferably 5 kDa to 18 kDa and 8 to 35%, further preferably 5 kDa to 18 kDa and 15 to 22%, from the viewpoint of improvement in retention in blood. The combination is preferably 5 kDa to 300 kDa and 2 to 30%, more preferably 5 kDa to 50 kDa and 2 to 22%, still more preferably 5 kDa to 27 kDa and 2 to 22%, even more preferably 5 kDa to 27 kDa and 7 to 22%, from the viewpoint of gelation.

Hyaluronic Acid Derivative Comprising Repeating Unit Represented by Formula (II)

In one aspect, the hyaluronic acid derivative comprising a repeating unit represented by the formula (II) is composed substantially of (1) a repeating unit of the formula (II); (2) repeating units of the formula (II) and the formula (III); (3) repeating units of the formula (II) and the formula (IIIc); or (4) repeating units of the formula (II), the formula (III), and the formula (IIIc). In the hyaluronic acid derivative, for example, 80% or more, preferably 90% or more, more preferably 95% or more, of disaccharide repeating units consisting of D-glucuronic acid and N-acetylglucosamine contained in the derivative are repeating units of the formula (II), (III), or (IIIc). In one aspect, the hyaluronic acid derivative is composed only of (1) a repeating unit of the formula (II); (2) repeating units of the formula (II) and the formula (III); (3) repeating units of the formula (II) and the formula (IIIc); or (4) repeating units of the formula (II), the formula (III), and the formula (IIIc).

The ratio of a particular disaccharide unit to disaccharide repeating units present in the hyaluronic acid derivative comprising a repeating unit represented by the formula (II) means the ratio of the particular disaccharide unit to all disaccharide units contained in a given amount of the hyaluronic acid derivative comprising a repeating unit represented by the formula (II), which is a polysaccharide having disaccharide units as repeating units.

In the formula (II) which represents a disaccharide unit contained in the hyaluronic acid derivative comprising a repeating unit represented by the formula (II), all of R^{1a}, R^{2a}, R^{3a}, and R^{4a} are preferably hydrogen atoms. R^{5a} is preferably a hydrogen atom or C₁₋₆ alkylcarbonyl, more preferably a hydrogen atom or acetyl, still more preferably acetyl. In the formulas (III) and (IIIc) which represent disaccharide units contained in the hyaluronic acid derivative comprising a repeating unit represented by the formula (II), all of R^{1b}, R^{2b}, R^{3b} and R^{4b}, and R^{1c}, R^{2c}, R^{3c} and R^{4c} are preferably hydrogen atoms. Each of R^{5b} and R^{5c} is preferably a hydrogen atom or C₁₋₆ alkylcarbonyl, more preferably a hydrogen atom or acetyl. Still more preferably, both of these moieties are acetyl.

Specific examples of R^{aa} in the formula (II) include a hydrogen atom, methyl, hydroxymethyl, 1-hydroxyethyl, carbamoylmethyl, carboxymethyl, 1-methylpropyl, 2-methylpropyl, isopropyl, 2-carboxyethyl, 2-methylthioethyl, 2-carbamoylethyl, phenylmethyl, (4-hydroxyphenyl)methyl, and indol-3-ylmethyl.

When the group —CHR^{aa}— serves as asymmetric center, respective optical isomers and their mixtures are also included therein. H₂N—CHR^{aa}—COOH (amino acid) preferably forms an L configuration (natural).

In the formula (II), R^{6a}, R⁷, R⁸, and R⁹ are each independently, for example, a hydrogen atom or methyl. Preferably, all of these moieties are hydrogen atoms.

The form of the group —CHR^{aa}—CO—X^{1a} in the formula (II) is, for example, a group —CHR^{aa}—COOH. Specific examples of the group include the following groups.

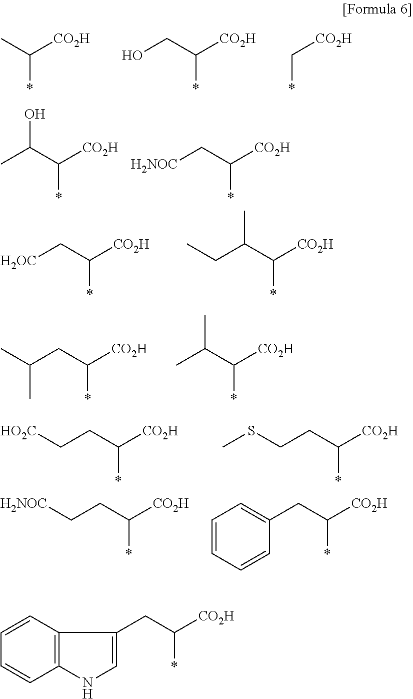

In this context, each asterisk represents a binding position to —NR^{6a}— (the same holds true for the description below).

Preferred examples of the group —CHR^{aa}—COOH include the following groups.

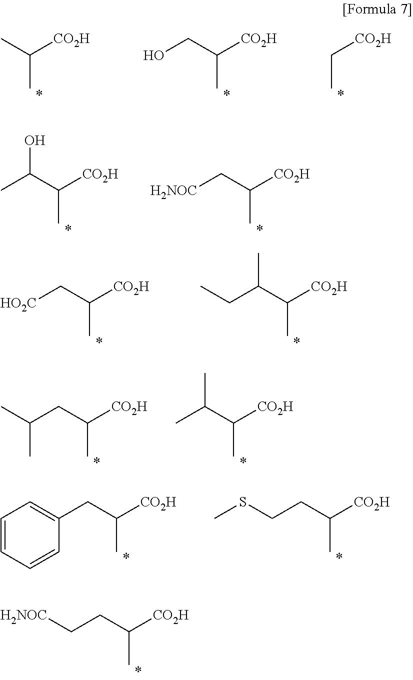

Preferred examples of the group —CHR^{aa}—COOH include the following groups.

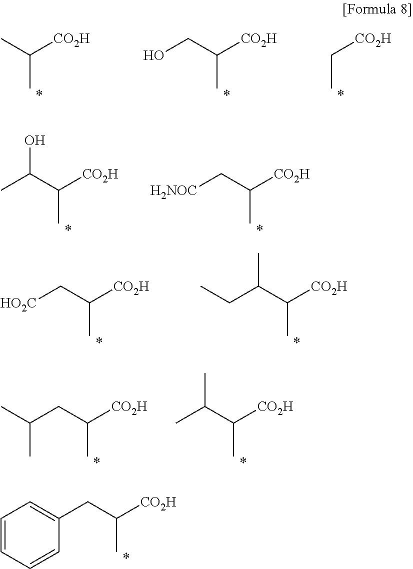

Preferred examples of the group —CHR^{aa}—COOH include the following groups.

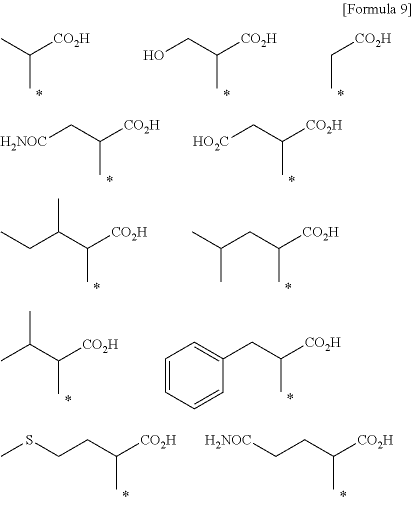

Preferred examples of the group —CHR^{aa}—COOH include the following groups.

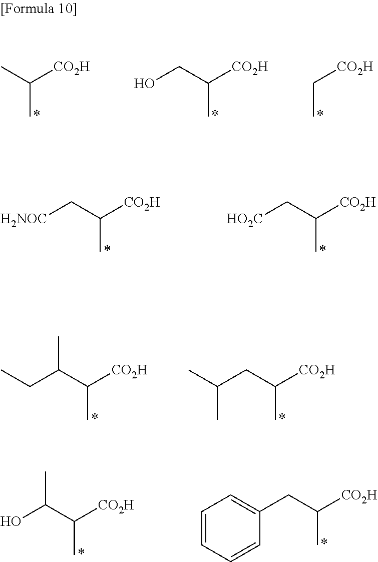

Preferred examples of the group —CHR^{aa}—COOH include the following groups from the viewpoint of the delivery of the complex to lymph node.

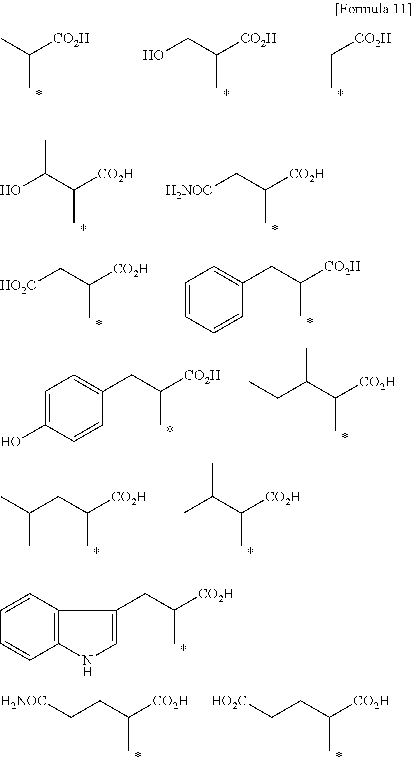

More preferred examples thereof include the following groups.

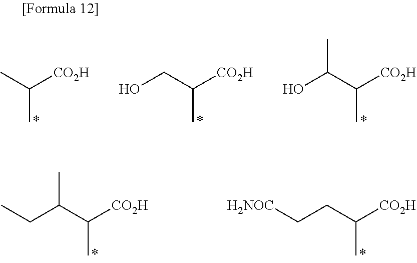

Still more preferred examples thereof include the following groups.

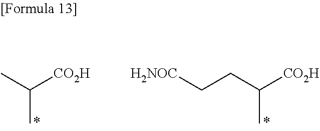

All the groups —CHR^{aa}—COOH mentioned above may be converted partially or wholly to a group —CHR^{aa}—CONH—Z¹—Z². Examples of the group —Z¹—Z² are as mentioned later.

Another form of the group —CHR^{aa}—CO—X^{1a} in the formula (II) is, for example, a group —CHR^{aa}—CONH₂. Specific examples of the group include the following groups.

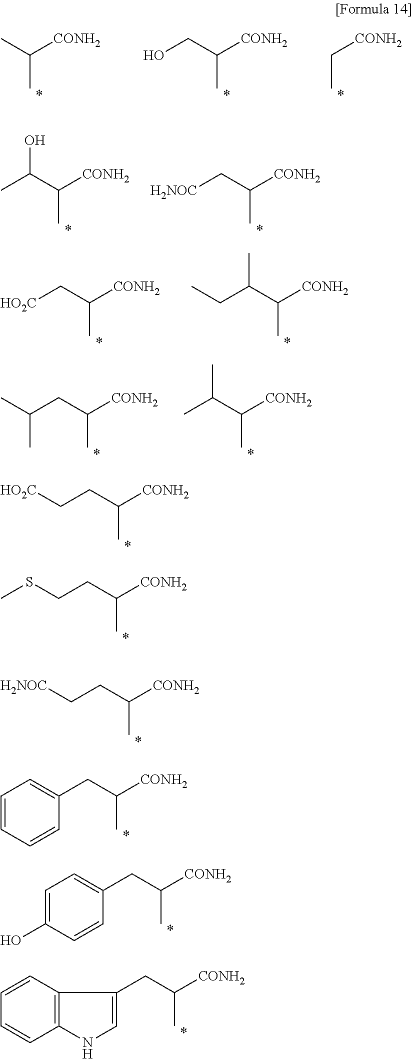

Preferred examples of the group —CHR^{aa}—CONH₂ include the following groups.

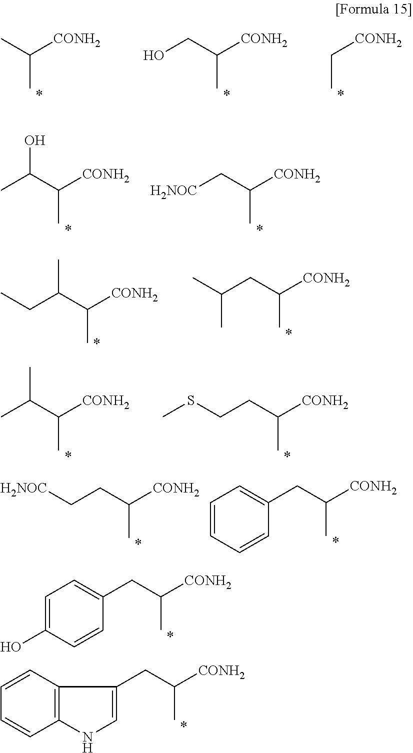

Preferred examples of the group —CHR^{aa}—CONH₂ include the following groups.

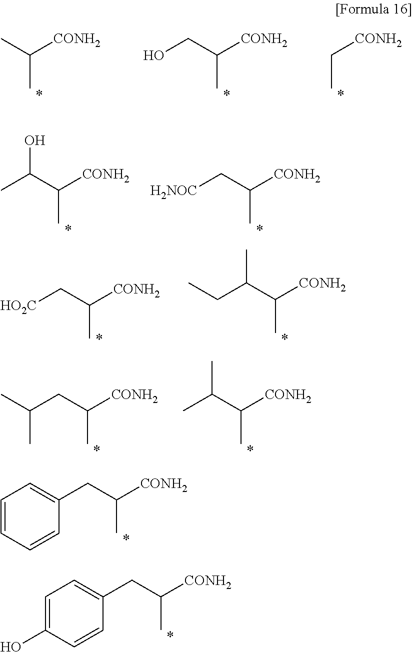

Preferred examples of the group —CHR^{aa}—CONH₂ include the following groups.

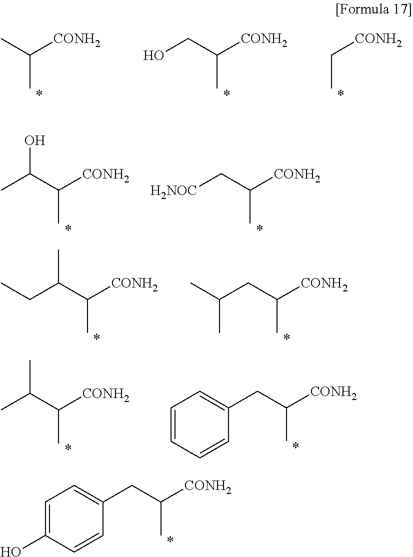

Preferred examples of the group —CHR^{aa}—CONH₂ include the following groups.

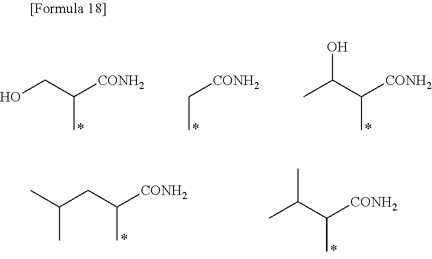

These groups are also preferred groups from the viewpoint of having both characteristics of biodegradability and retention in blood.

Preferred examples of the group —CHR^{aa}—CONH₂ also include the following groups from the viewpoint of having both characteristics of biodegradability and retention in blood.

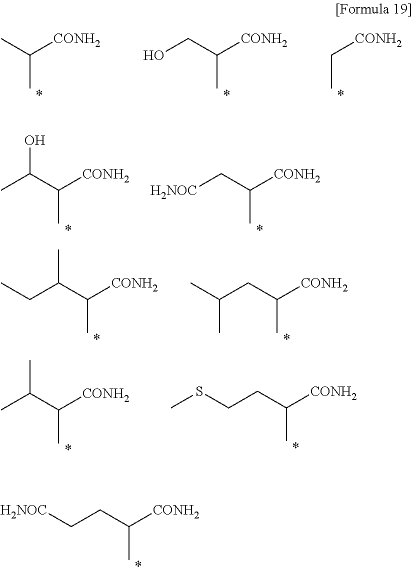

More preferred examples of the group —CHR^{aa}—CONH₂ also include the following groups from the viewpoint of having both characteristics of biodegradability and retention in blood.

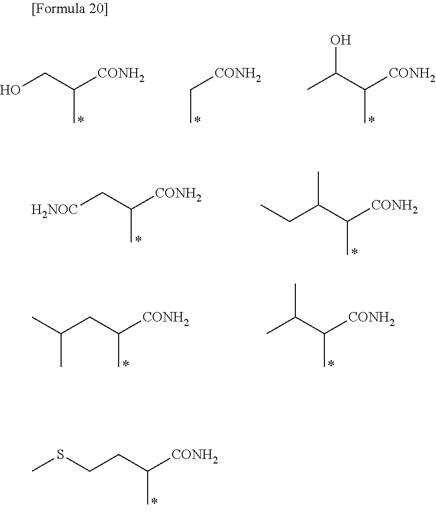

Preferred examples of the group —CHR^{aa}—CONH₂ include the following groups from the viewpoint of more suitable dispersibility in pure water.

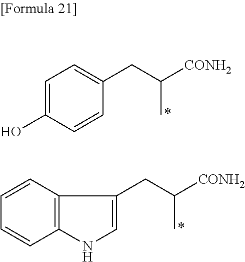

These two groups are also preferred examples from the viewpoint of a base material for an injection formulation for continuous subcutaneous administration.

Preferred examples of the group —CHR^{aa}—CONH₂ include the following group from the viewpoint of a base material for an injection formulation for continuous subcutaneous administration.

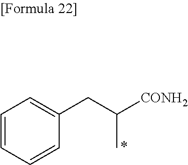

R⁷ is more preferably a hydrogen atom or methyl, still more preferably a hydrogen atom.

The carboxy defined in the formulas (II), (III) and (IIIc) may form a salt represented by the formula —COO⁻Q⁺. In this context, Q⁺ is not particularly limited as long as Q⁺ is a counter cation that forms a salt with carboxy in water. Divalent or higher valent Q⁺ forms a salt with a plurality of carboxy groups according to the valence. Examples of the counter cation include: metal ions such as lithium ions, sodium ions, rubidium ions, cesium ions, magnesium ions, and calcium ions; and ammonium ions represented by the formula: N⁺R^{j}R^{k}R^{l}R^{m} (wherein R^{j}, R^{k}, R^{l} and R^{m} are each independently selected from a hydrogen atom and C₁₋₆ alkyl), and preferably include sodium ions, potassium ions, and tetraalkylammonium ions (e.g., tetra-n-butylammonium ions). R^{j}, R^{k}, R^{l} and R^{m} are preferably the same groups selected from C₁₋₆, alkyl, and are preferably n-butyl groups.

An alternative form of the group —CHR^{aa}—CO—X^{1a} in the formula (II) is, for example, a group —CHR^{aa}—CONH—Z¹—Z². Specific examples of the group include the following groups.

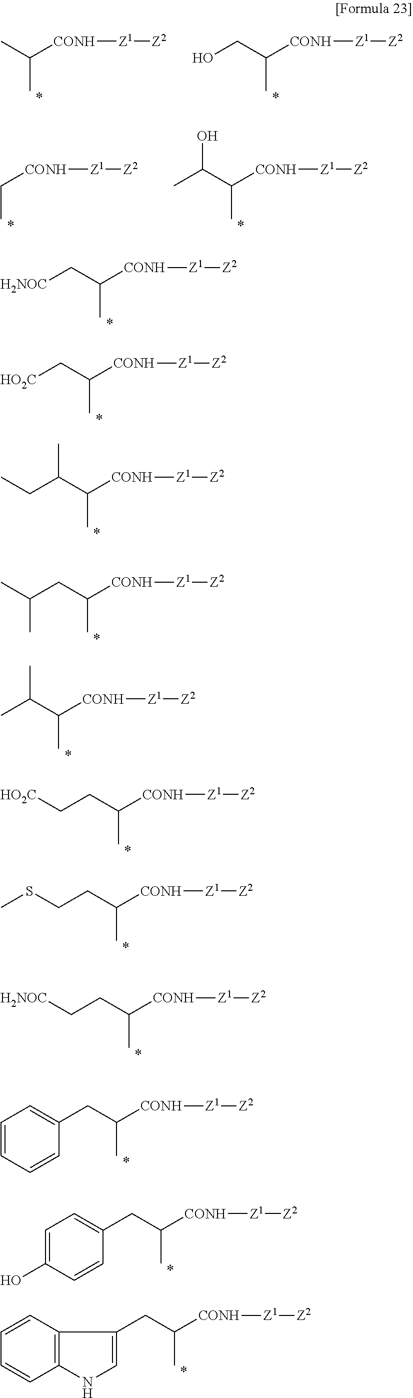

Other specific examples of the group include the following groups.

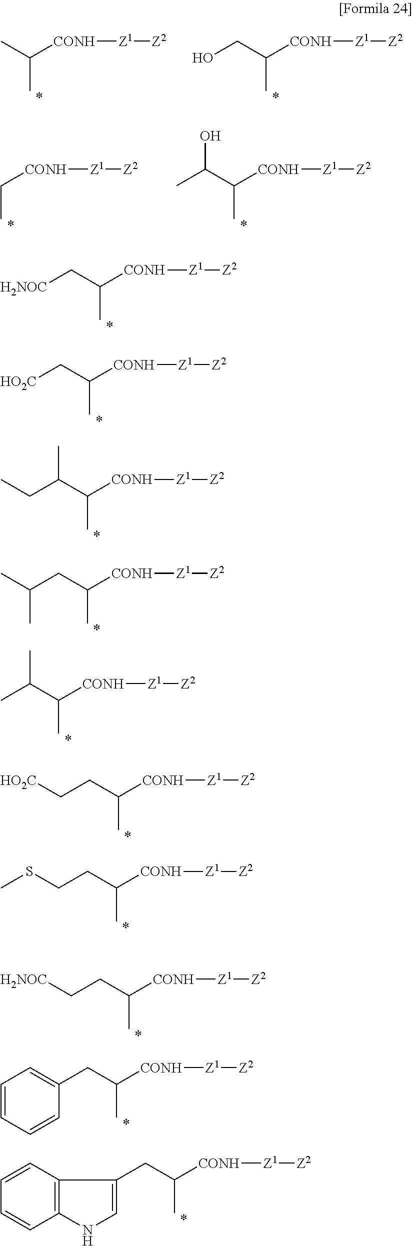

Preferred examples of the group —CHR^{aa}—CONH—Z¹—Z² include the following groups.

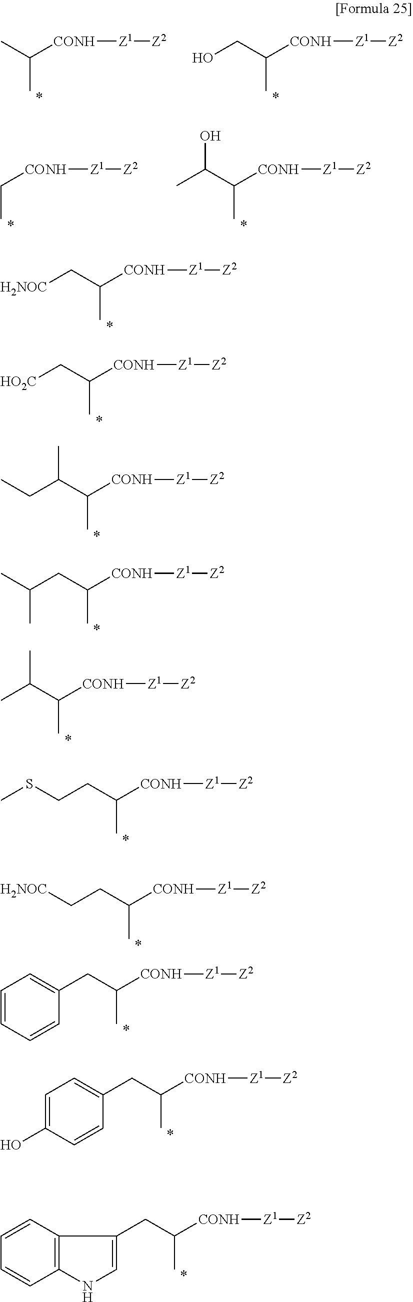

Preferred examples of the group —CHR^{aa}—CONH—Z¹—Z² include the following groups.

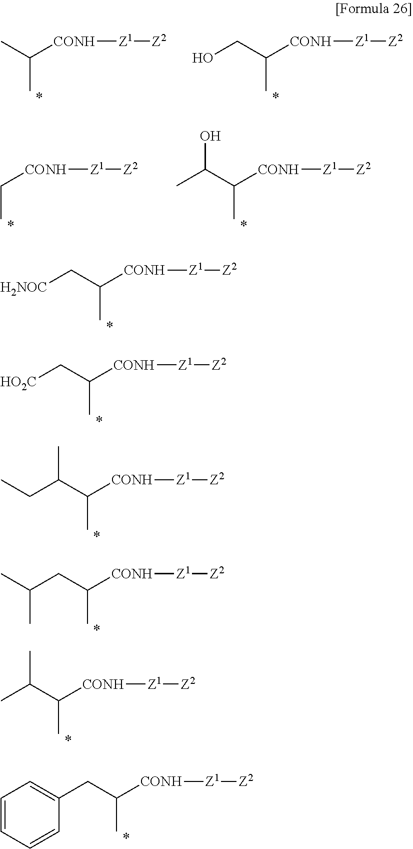

Preferred examples of the group —CHR^{aa}—CONH—Z¹—Z² include the following groups.

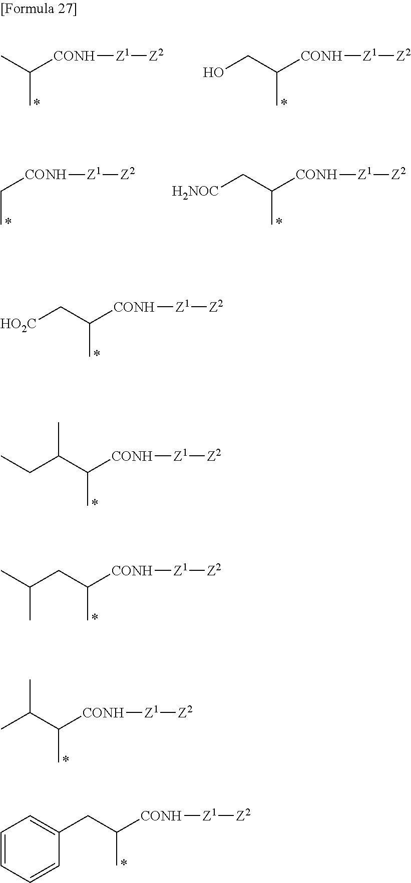

Preferred examples of the group —CHR^{aa}—CONH—Z¹—Z² include the following groups from the viewpoint of the delivery of the complex to lymph node.

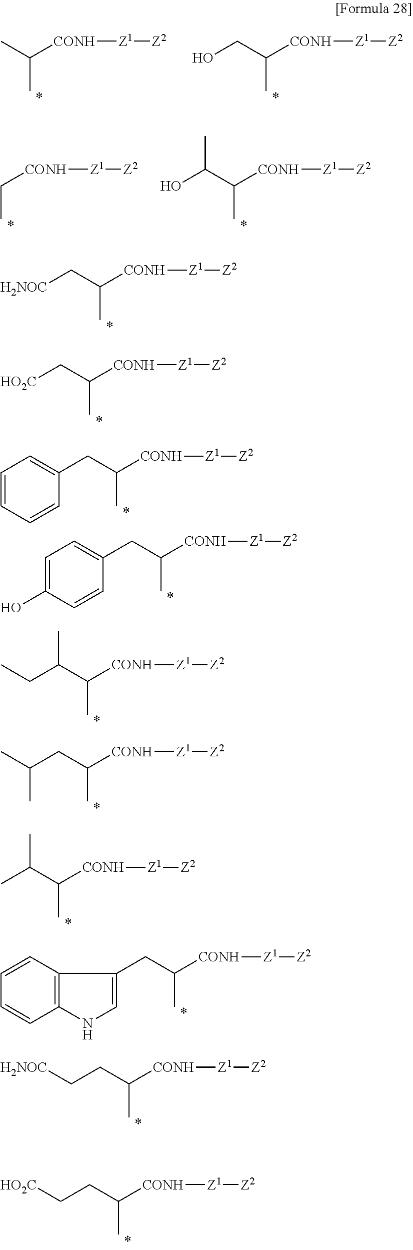

More preferred examples of the group include the following groups.

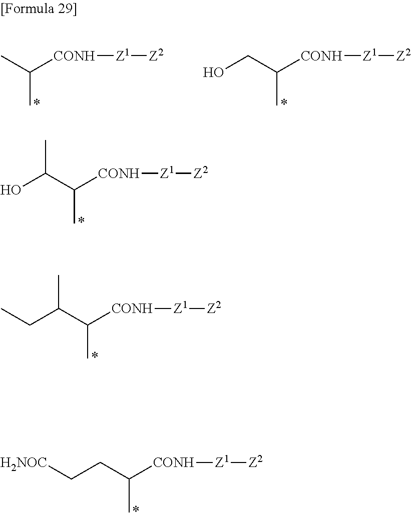

Still more preferred examples thereof include the following groups.

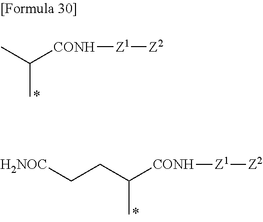

Preferred examples of the group —CHR^{aa}—CONH—Z¹—Z² include the following groups from the viewpoint of having both characteristics of biodegradability and retention in blood.

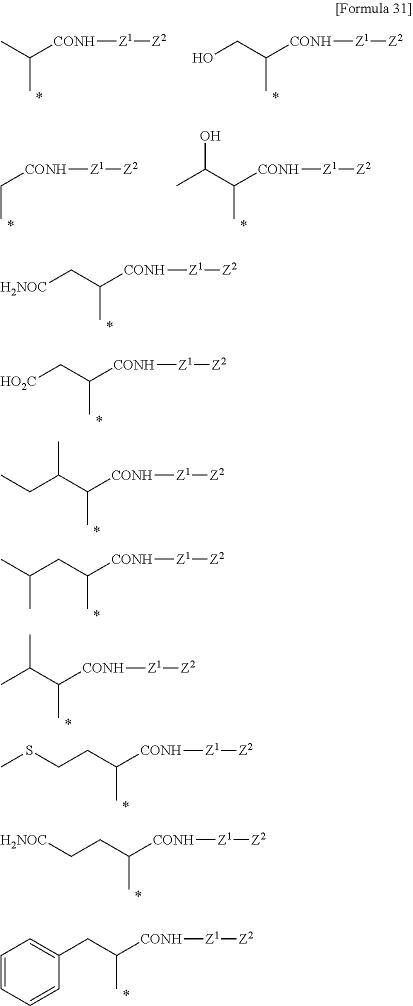

Examples of the group —Z¹—Z² include a group —(C₂₋₁₀ alkylene)-NH—COO—Z³. Examples thereof also include a group —(C₂₋₁₂ alkylene)-NH—COO—Z³. In this context, examples of the C₂₋₁₂ alkylene preferably include —(CH₂)₂—, —(CH₂)₆—, —(CH₂)₈—, —(CH₂)₁₀— and —(CH₂)₁₂— and more preferably include —(CH₂)₂— and —(CH₂)₆—. Examples of the group —Z¹—Z² include a group —(CH₂CH₂O)ₘₐ—CH₂CH₂—NH—Z³. In this context, ma is preferably 1 to 20, more preferably 1 to 10, still more preferably 1 to 3. Specific examples of preferred ma include 2. Examples of the group —Z¹—Z² preferably include a group -(hexane-1,6-diyl)-NH—COO—Z³, a group -(ethane-1,2-diyl)-NH—COO—Z³ and a group —(CH₂CH₂O)₂—CH₂CH₂—NH—Z³, more preferably include a group -(hexane-1,6-diyl)-NH—COO-cholesteryl, a group -(ethane-1,2-diyl)-NH—COO-cholesterol and a group-(CH₂CH₂O)₂—CH₂CH₂—NH-cholanoyl, and still more preferably include a group -(hexane-1,6-diyl)-NH—COO-cholesteryl. Examples of Z¹, Z², and the group —Z¹—Z² also include those corresponding to Y, X¹, and the group-Y—X¹, respectively, in the hyaluronic acid derivative comprising a repeating unit represented by the formula (I). Examples of the group —CO—NR^{ca}—Z³ and the group —O—CO—NR^{ca}—Z³ include respective groups wherein R^{ca} is a hydrogen atom.

In one aspect, in the hyaluronic acid derivative comprising a repeating unit represented by the formula (II), the group X^{1a} is —NR⁹—Z¹—Z², R⁹ is a hydrogen atom. Z¹ is, for example, C₂₋₁₂ alkylene, preferably C₂₋₆ alkylene, more preferably C₆ alkylene, Z² is —NR^{ba}—COO—Z³, R is a hydrogen atom, and Z³ is a steryl group; and the group R^{aa} is a hydrogen atom, or C₁₋₆ alkyl, wherein the alkyl is optionally substituted by one or more groups each independently selected from hydroxy, carboxy, carbamoyl, C₁₋₆ alkylthio, aryl, and heteroaryl, wherein the aryl is optionally substituted by one or more hydroxy groups.

Specific examples of the group R^{aa} preferably include methyl, hydroxymethyl, a hydrogen atom, 1-hydroxyethyl, carbamoylmethyl, carboxymethyl, benzyl, (4-hydroxyphenyl)methyl, 1-methylpropyl, 2-methylpropyl, isopropyl, indol-3-ylmethyl, 2-carbamoylethyl and 2-carboxyethyl, more preferably include methyl, hydroxymethyl, 1-hydroxyethyl, 1-methylpropyl and 2-carbamoylethyl, still more preferably include methyl and 2-carbamoylethyl, from the viewpoint of the delivery of the complex to lymph node.

In one aspect, the hyaluronic acid derivative comprising a repeating unit represented by the formula (II) preferably also comprises a repeating unit represented by the formula (III). In a more preferred aspect, X^{1a} in the formula (II) and X² in the formula (III) are the same. In one aspect, the hyaluronic acid derivative comprising a repeating unit represented by the formula (II) may comprise a repeating unit represented by the formula (II), a repeating unit represented by the formula (III) and a repeating unit represented by the formula (IIIc), wherein X^{1a} is —NR⁹—Z¹—Z².

According to a further alternative aspect, in the hyaluronic acid derivative comprising a repeating unit represented by the formula (II), the ratio of the repeating unit of the formula (II) and/or the formula (III) having a group —NR⁹—Z¹—Z² (hereinafter, also referred to as a hydrophobic group) to disaccharide repeating units present (rate of introduction of the hydrophobic group) may be 3 to 50%.

In this context, the rate of introduction of the hydrophobic group is calculated according to the following expression:

$$( Rate of introduction of hydrophobic group ) = \frac{\begin{matrix}( The number of disaccharide repeating units \\ having an introduced hydrophobic group )\end{matrix}}{\begin{matrix}( The number of disaccharide \\ repeating units present )\end{matrix} } \times 100 \qquad [ Equation 2 ]$$

In this context, the “disaccharide repeating units present in the derivative” include repeating units of the formula (II) and the formula (III), and a repeating unit of the formula (IIIc). The rate of introduction can be controlled by reaction conditions, for example, the ratio of a reagent, and can be determined by, for example, NMR measurement.

The rate of introduction of the hydrophobic group in the hyaluronic acid derivative comprising a repeating unit represented by the formula (II) is, for example, 3 to 50%, preferably 5 to 40%, more preferably 5 to 35%, still more preferably 5 to 25%, even more preferably 5 to 20%, further preferably 5 to 10%. The rate is preferably 3 to 60%, more preferably 7 to 50%, still more preferably 18 to 45%, even more preferably 20 to 35%, from the viewpoint of complex formation with the antigen. The rate is preferably 3 to 60%, more preferably 7 to 50%, still more preferably 18 to 45%, even more preferably 20 to 35%, from the viewpoint of the transfer of the complex to lymph node.

In one aspect, in the hyaluronic acid derivative comprising a repeating unit represented by the formula (II), X^{1a} in the formula (II) is —NR⁹—Z¹—Z². In this case, the ratio of the disaccharide unit of the formula (II) to disaccharide repeating units present in the hyaluronic acid derivative is, for example, 70% or more, preferably 75% or more, more preferably 90% or more, from the viewpoint of having both characteristics of biodegradability and retention in blood. The upper limit can be 100% or less. The range of the ratio is, for example, 70 to 100%, preferably 75 to 100%, more preferably 90 to 100%. The ratio is, for example, 10% or more, preferably 20% or more, more preferably 50% or more, still more preferably 70% or more, even more preferably 90% or more, from the viewpoint of having characteristics of complex formation with the antigen. The upper limit can be 100% or less. The range of the ratio is, for example, 10 to 100%, preferably 20 to 100%, more preferably 50 to 100%, still more preferably 70 to 100%, even more preferably 90 to 100%. The ratio is, for example, 10% or more, preferably 20% or more, more preferably 50% or more, still more preferably 70% or more, even more preferably 90% or more, from the viewpoint of having characteristics of transfer of the complex to lymph node. The upper limit can be 100% or less. The range of the ratio is, for example, 10 to 100%, preferably 20 to 100%, more preferably 50 to 100%, still more preferably 70 to 100%, even more preferably 90 to 100%. The hyaluronic acid derivative comprising a repeating unit represented by the formula (II) may further comprise a repeating unit represented by the formula (III).

In one aspect, the hyaluronic acid derivative comprising a repeating unit represented by the formula (II) does not comprise a repeating unit represented by the formula (II) wherein X¹ is —NR⁹—Z¹—Z². In this case, the sum of the ratio of the repeating unit represented by the formula (II), and the ratio of the repeating unit represented by the formula (III) to disaccharide repeating units present is, for example, 70 to 100%, preferably 80 to 100%, more preferably 90 to 100%. The sum is, for example, 7 to 100%, preferably 20 to 100%, more preferably 30 to 100%, from the viewpoint of complex formation with the antigen. The sum is, for example, 20 to 100%, preferably 30 to 100%, more preferably 70 to 100%, from the viewpoint of the transfer of the complex to lymph node.

In this context, in the hyaluronic acid derivative comprising a repeating unit represented by the formula (II), the ratio of the repeating unit represented by the formula (III) to disaccharide repeating units present is preferably 3 to 50%, more preferably 5 to 40%, still more preferably 5 to 35%, even more preferably 5 to 25%, further preferably 5 to 20%, still further preferably 5 to 10%. The ratio is preferably 3 to 60%, more preferably 7 to 50%, still more preferably 18 to 45%, even more preferably 20 to 35%, from the viewpoint of complex formation with the antigen. The ratio is preferably 3 to 60%, more preferably 7 to 50%, still more preferably 18 to 45%, even more preferably 20 to 35%, from the viewpoint of the transfer of the complex to lymph node.

In the hyaluronic acid derivative comprising a repeating unit represented by the formula (II), the ratio of the repeating unit represented by the formula (II) to disaccharide repeating units present is preferably 20 to 97%, more preferably 30 to 95%, still more preferably 35 to 95%, even more preferably 45 to 95%, further preferably 50 to 95%, still further preferably 60 to 95%. The ratio is, for example, 7 to 100%, preferably 20 to 100%, more preferably 30 to 100%, from the viewpoint of complex formation with the antigen. The ratio is, for example, 20 to 100%, preferably 30 to 100%, more preferably 70 to 100%, from the viewpoint of the transfer of the complex to lymph node.

Examples of the methods for producing the hyaluronic acid derivative comprising a repeating unit represented by the formula (I) and the hyaluronic acid derivative comprising a repeating unit represented by the formula (II) include methods described in International Publication Nos. WO 2010/053140 and WO 2014/038641.

According to one aspect, a feature of the hyaluronic acid derivative comprising a repeating unit represented by the formula (I) or the hyaluronic acid derivative comprising a repeating unit represented by the formula (II) is that the hyaluronic acid derivative forms a fine particle by association in water. It is considered that spontaneous association occurs in water through the hydrophobic interaction of the introduced hydrophobic group so that a fine particle is formed, though the event is not particularly limited thereto. By exploiting such characteristics, the hyaluronic acid derivative can be used as a carrier for a vaccine, a lymph node delivery carrier, a sustained-release carrier in blood, or a targeting carrier. The particle size of the fine particle is not particularly limited and is, for example, 1 μm or smaller, preferably 500 nm or smaller, more preferably 200 nm or smaller, still more preferably 100 nm or smaller, even more preferably 50 nm or smaller. Examples of the methods for forming the fine particles of the hyaluronic acid derivative comprising a repeating unit represented by the formula (I) and the hyaluronic acid derivative comprising a repeating unit represented by the formula (II) include methods described in International Publication Nos. WO 2010/053140 and WO 2014/038641.

Hyaluronic acid or a salt thereof or a derivative thereof can be used as a starting material for producing the hyaluronic acid derivative comprising a repeating unit represented by the formula (I) and the hyaluronic acid derivative comprising a repeating unit represented by the formula (II). Examples of the hyaluronic acid salt can include alkali metal salts such as sodium salts, potassium salts, and lithium salts. The salt is particularly preferably a sodium salt which is frequently used as a medicament. The hyaluronic acid or a pharmaceutically acceptable salt thereof can be produced by use of various methods known in the art, such as a method of obtaining organism-derived extracts from cock's comb, under the swine skin, etc., and a biological fermentation method, or may be obtained by purchasing a commercially available product (e.g., from Denka Co., Ltd., Shiseido Co., Ltd., Seikagaku Corp., and R&D Systems, Inc.).

The antigen in the complex formed with the fine particle of the hyaluronic acid derivative comprising a repeating unit represented by the formula (I) or the hyaluronic acid derivative comprising a repeating unit represented by the formula (II) may be released from the complex by the degradation or disruption of the fine particle of the hyaluronic acid derivative, and the replacement of the antigen with a biocomponent such as albumin, after administration. The velocity of this release may be controlled by the rate of introduction of the hydrophobic group, molecular weight, or rate of amino acid introduction of the hyaluronic acid derivative. The antigen release velocity may be controlled by the chemical cross-linking and gelation of the hyaluronic acid derivative according to a method described in International Publication No. WO 2010/053140. Also, the antigen release velocity may be controlled by conjugating the hyaluronic acid derivative with the antigen according to a method described in International Publication No. WO 2010/053140.

Antigen

Examples of the antigen that is used for producing a vaccine formulation by forming a complex with the hyaluronic acid derivative comprising a repeating unit represented by the formula (I) or the hyaluronic acid derivative comprising a repeating unit represented by the formula (II) include antigenic peptides and antigenic proteins, and DNA and mRNA encoding any of these antigen sequences. The antigen is preferably an antigenic peptide or an antigenic protein, more preferably an antigenic peptide.

The antigen may be a cancer antigen. The cancer antigen is an antigen often expressed in cancer cells and, in some cases, is expressed only by cancer cells. The cancer antigen may be expressed within cancer cells or on the surface of cancer cells.

The antigenic protein that may be used in the present invention may be selected from, but not limited to, MART-1/Melan-A, gp100, adenosine deaminase binding protein (ADAbp), FAP, cyclophilin b, colorectal associated antigen (CRC)-C017-1A/GA733, carcinoembryonic antigen (CEA), CAP-1, CAP-2, etv6, AML1, prostate-specific antigen (PSA), P SA-1, PSA-2, PSA-3, prostate-specific membrane antigen (PSMA), T cell receptor/CD3-zeta chain, CD20, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5, GAGE-1, GAGE-2, GAGE-3, GAGE-4, GAGE-5, GAGE-6, GAGE-7, GAGE-8 and GAGE-9, RAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin, γ-catenin, p120ctn, gp100Pmel117, PRAME, NY-ESO-1, cdc27, adenomatous polyposis coli protein (APC), fodrin, connexin 37, Ig idiotype, p15, gp75, GM2 ganglioside, GD2 ganglioside, human papillomavirus protein, tumor antigen Smad family, Imp-1, P1A, nuclear antigen encoded by EBV (EBNA)-1, brain glycogen phosphorylase, SSX-1, SSX-2(HOM-MEL-40), SSX-1, SSX-4, SSX-5, SCP-1, CT-7, CD20 and c-erbB-2.

The whole sequence of the antigenic protein may be used, or a sequence thereof having a partial deletion may be used.

The antigenic peptide is an antigenic peptide comprising one or more CD8-positive cytotoxic T cell recognition epitopes and/or CD4-positive helper T cell recognition epitopes in the sequences of antigenic proteins. In one aspect, the antigenic peptide is an antigenic peptide preferably comprising two or more epitopes from the viewpoint of being loaded to a MHC class I molecule or a MHC class II molecule through degradation in antigen-presenting cells. Specifically, examples of the antigenic peptide include antigenic peptides comprising antigenic protein epitopes of tumor cells.

In one aspect, the antigenic peptide has, for example, 8 to 120 amino acids, preferably 8 to 80 amino acids, more preferably 15 to 80 amino acids, still more preferably 16 to 80 amino acids, even more preferably 23 to 80 amino acids, further preferably 23 to 60 amino acids, still further preferably 23 to 50 amino acids.

In one aspect, the antigenic peptide is an antigenic peptide comprising one or more each of CD8-positive cytotoxic T cell recognition epitopes and CD4-positive helper T cell recognition epitopes from the viewpoint of inducing the activation of cytotoxic T cells (CTL) by helper T cells.

In one aspect, when two or more epitopes are contained, an amino acid linker may be disposed between the epitopes. The linker has, for example, 2 to 10 amino acids, preferably 4 to 10 amino acids, more preferably 4 to 8 amino acids. Examples of the amino acids for use in the linker include glycine (G), tyrosine (Y), leucine (L), and tryptophan (W). The amino acids are preferably tyrosine (Y), leucine (L), or tryptophan (W). Specific examples of the amino acid linker include -YYYY- (4Y), -LLLL- (4L), -WWWW- (4W), -GGGGGG- (6G), -YYYYYY- (6Y), -LLLLLL- (6L), -WWWWWW- (6W), -YYYYYYYY- (8Y), -LLLLLLLL- (8L) and -WWWWWWWW- (8W). 6Y, 6L and 6W are preferred.

Complex

The complex of a hyaluronic acid derivative having an introduced hydrophobic group, and an antigen according to the present invention can be produced by mixing a hyaluronic acid derivative comprising a repeating unit represented by the formula (I) or a hyaluronic acid derivative comprising a repeating unit represented by the formula (II) with an antigen in an appropriate solution. Examples of the solution used include water, saline, various buffer solutions, sugar solutions, DMSO, ethanol, DMF, and combinations thereof. Solvent or buffer solution replacement may be performed by a method such as dialysis.

The complex of the present invention can be produced, for example, by mixing a fine particle of a hyaluronic acid derivative comprising a repeating unit represented by the formula (I) or a hyaluronic acid derivative comprising a repeating unit represented by the formula (II) with an antigen. The fine particle of the hyaluronic acid derivative and the antigen form a complex, which can be used as the complex of the hyaluronic acid derivative and the antigen, though the event is not particularly limited thereto. Alternatively, the complex of the hyaluronic acid derivative and the antigen can be formed by enclosing the antigen in the fine particle of the hyaluronic acid derivative. The complex includes the antigen enclosed in the fine particle of the hyaluronic acid derivative. In the antibody enclosed therein, the antigen is coated with the hyaluronic acid derivative.

Examples of the complex formation method include a method of adding an antigen solution to a fine particle formed in advance, of a hyaluronic acid derivative comprising a repeating unit represented by the formula (I) or a hyaluronic acid derivative comprising a repeating unit represented by the formula (II). In this method, the formed fine particle of the hyaluronic acid derivative and the antigen form a complex through interaction such as hydrophobic interaction, electrostatic interaction, or a hydrogen bond. The interaction occurs on the surface of the fine particle or in the inside of the fine particle. Conditions such as a solvent, a salt concentration, pH, a temperature, a time, and the addition of a denaturant are not particularly limited and can be appropriately selected such that the antigen forms a complex stably at high yields. Depending on, for example, a salt concentration or pH at the time of complex formation, the degree of swelling or density of the fine particle of the hyaluronic acid derivative varies, and the ionization state of the antigen also varies. Therefore, suitable conditions can be appropriately used according to a combination thereof. When the complex is formed under a low salt concentration, a fine particle density can be decreased and the amount of the complex formed can be increased through the use of electrostatic repulsion between carboxyl groups of hyaluronic acid derivatives, or a complex with a higher-molecular-weight antigen can be formed. After complex formation, the electrostatic repulsion is weakened by the elevation of the salt concentration. The fine particle density is thereby elevated so that gel networks are smaller than the antigen size. As a result, the antigen is firmly retained, and its release can be delayed. In this respect, the salt concentration may be set to a physiological salt concentration. Ultrasonic irradiation may be performed using an ultrasonic homogenizer or a single-point focused ultrasonic irradiation apparatus for miniaturization or size uniformization. The ultrasonic irradiation may be performed after mixing of the hyaluronic acid derivative with the antigen or may be performed only on the hyaluronic acid derivative. Free uncomplexed antigens can be separated and removed by a dialysis method or size exclusion chromatography (SEC).

In another complex formation method, the hyaluronic acid derivative comprising a repeating unit represented by the formula (I) or the hyaluronic acid derivative comprising a repeating unit represented by the formula (II) can be dissolved in an aprotic polar organic solvent such as DMSO or DMF and mixed with the antigen, followed by replacement with water, an aqueous salt solution, or any of various aqueous solutions of buffer solutions to perform fine particle formation and complex formation at the same time. The replacement can be performed by, for example, dialysis. Ultrasonic irradiation may be performed using an ultrasonic homogenizer or a single-point focused ultrasonic irradiation apparatus for miniaturization or size uniformization. The ultrasonic irradiation may be performed after mixing of the hyaluronic acid derivative with the antigen or may be performed only on the hyaluronic acid derivative. Also, the ultrasonic irradiation may be performed before or after dialysis. The antigen complexed with the hyaluronic acid derivative by this method is less likely to be released from complex. Conditions for mixing the hyaluronic acid derivative with the antigen, such as a solvent species, a salt concentration, pH, a temperature, a time, the addition of a denaturant, a hyaluronic acid derivative concentration, an antigen concentration, and the ratio between the hyaluronic acid derivative and the antigen are not particularly limited and can be appropriately selected such that the antigen forms a complex stably at high yields. Conditions for replacement with water, an aqueous salt solution, or any of various aqueous solutions of buffer solutions, such as a solvent species, a salt concentration, pH, a temperature, a time, the number of runs, the presence or absence of addition of a denaturant, a hyaluronic acid derivative concentration, an antigen concentration, and the ratio between the hyaluronic acid derivative and the antigen are not particularly limited and can be appropriately selected such that the antigen forms a complex stably at high yields. The size of the fine particles is also obtained as intended by appropriately selecting these conditions. Free uncomplexed antigens can be separated and removed by a dialysis method or size exclusion chromatography (SEC).

When the complex is a particle, its particle size is not particularly limited and is, for example, 20 to 200 nm, preferably 20 to 100 nm, more preferably 20 to 50 nm, from the viewpoint of transfer to lymph node.

Vaccine Formulation

The complex can be used to produce the vaccine formulation of the present invention, specifically, the vaccine formulation for use in the prevention and/or treatment of a cancer.

The vaccine formulation of the present invention may be administered through an oral, extraintestinal, intranasal, intravaginal, intraocular, subcutaneous, intravenous, intramuscular, intracutaneous, intraperitoneal, joint cavity, intracerebral or intraoral route. Preferably, the vaccine formulation may be administered subcutaneously, intramuscularly, intravenously, or intracutaneously.

The vaccine formulation of the present invention can be administered as a pharmaceutical composition comprising one or more pharmaceutically acceptable additives such as diluent, a wetting agent, an emulsifier, a dispersant, an aid, an antiseptic, a buffer, a binder, and/or a stabilizer, in any appropriate form according to an intended administration route. The administration route may be a parenteral route or may be an oral route.

The vaccine formulation of the present invention may be administered in combination with at least one type of adjuvant. The adjuvant used can be a substance having an effect of potentiating the activity of antigen-presenting cells and is more specifically selected from a substance activating an innate immune receptor (pattern recognition receptor) and other antigen-presenting cell-stimulating substances, and a substance having an effect of inhibiting the acquirement of immunosuppressive activity by antigen-presenting cells. The innate immune receptor is classified into Toll-like receptor (TLR), C-type lectin receptor (CLR), NOD-like receptor (NLR), RIG-I-like receptor (RLR) and cytoplasmic DNA sensor. The adjuvant which is an innate immune receptor agonist can be selected from inactivated microbial cells, a microbial cell extract, a nucleic acid, a lipopolysaccharide, a lipopeptide, a synthetic low-molecular compound, and the like. Preferably, CpG oligo DNA, polyIC RNA, imidazoquinoline (e.g., R848 and imiquimod), saponin (e.g., QuilA and QS21), a STING agonist (e.g., cyclic di-GMP), monophosphoryl lipid, a lipopeptide, or the like is used. A taxane drug or an anthracycline drug can be used as the adjuvant which is an antigen-presenting cell-stimulating agent. The adjuvant which is an agent having an effect of inhibiting the acquirement of immunosuppressive activity by antigen-presenting cells is selected from a substance inhibiting JAK/STAT, a substance inhibiting indole dioxygenase (IDO), a substance inhibiting tryptophan dioxygenase (TDO), and the like. These inhibiting substances include compounds having an antagonistic effect on the factors as well as neutralizing antibodies, small interfering RNA (siRNA), and antisense DNA against the factors.

The adjuvant that is used for the vaccine formulation of the present invention is preferably an adjuvant which is an innate immune receptor agonist, more preferably an agonist of Toll-like receptor (TLR) or cytoplasmic DNA sensor. The TLR agonist is preferably CpG oligo DNA, polyIC RNA, imidazoquinoline such as R848 or imiquimod, saponin such as QuilA or QS21, a STING agonist, or monophosphoryl lipid.

The adjuvant and the vaccine formulation may be administered as separate formulations or may be administered as one formulation. The adjuvant may be conjugated, for use, with the hyaluronic acid derivative comprising a repeating unit represented by the formula (I) by a method described in International Publication No. WO 2010/053140. For administration in combination, the amount of the vaccine formulation of the present invention administered is, for example, 0.01 to 100 mg/dose, preferably 0.1 to 50 mg/dose, more preferably, for example, 0.1 to 20 mg/dose, and the amount of the adjuvant administered is, for example, 0.01 to 100 mg/kg body weight, preferably 0.1 to 50 mg/kg body weight, more preferably 0.1 to 10 mg/kg body weight. The adjuvant may be administered at the same timing as in the vaccine formulation of the present invention (including the case where the adjuvant is contained in the vaccine formulation), or the adjuvant and the vaccine formulation may be administered at different timings. For administration at different timings, preferably, one of the adjuvant and the vaccine formulation is administered, and the other is administered, for example, within 1 minute to 5 hours, preferably within 1 minute to 1 hour, thereafter.

The vaccine formulation of the present invention may be administered in combination with at least one type of antibody for use in cancer treatment. The antibody is, for example, an antibody inhibiting an immunosuppressive signal from tumor, or at least one type of antibody activating a costimulatory signal of immunocytes, preferably an antibody inhibiting an immunosuppressive signal from tumor or an antibody activating a costimulatory signal of immunocytes. Specifically, the antibody is at least one type of antibody selected from an anti-CTLA4 antibody, an anti-PD1 antibody, an anti-PDL1 antibody, an anti-OX40 antibody, and an anti-4-1BB antibody. For administration in combination, the amount of the vaccine formulation of the present invention administered is, for example, 0.01 to 100 mg/dose, preferably 0.1 to 50 mg/dose, more preferably 0.1 to 20 mg/dose, and the amount of the antibody administered is, for example, 0.01 to 200 mg/kg body weight, preferably 0.1 to 100 mg/kg body weight, more preferably 1 to 40 mg/kg body weight. The antibody may be administered at the same timing as in the vaccine formulation of the present invention (including the case where the antibody is contained in the vaccine formulation), or the antibody and the vaccine formulation may be administered at different timings. For administration at different timings, preferably, one of the antibody and the vaccine formulation is administered, and the other is administered, for example, within 1 minute to 24 hours, preferably within 1 minute to 5 hours, thereafter.

The vaccine formulation of the present invention may be administered in combination with both the adjuvant and the antibody. In this case, the amount of the vaccine formulation of the present invention administered is, for example, 0.01 to 100 mg/dose, preferably 0.1 to 50 mg/dose, more preferably, for example, 0.1 to 20 mg/dose, the amount of the adjuvant administered is, for example, 0.01 to 100 mg/kg body weight, preferably 0.1 to 50 mg/kg body weight, more preferably 0.1 to 10 mg/kg body weight, and the amount of the antibody administered is, for example, 0.01 to 200 mg/kg body weight, preferably 0.1 to 100 mg/kg body weight, more preferably 1 to 40 mg/kg body weight. The adjuvant and the antibody may be administered at the same timing as in the vaccine formulation of the present invention (including the case where the adjuvant and the antibody are contained in the vaccine formulation), or the adjuvant and the antibody and the vaccine formulation may be administered at different timings. For administration at different timings, preferably, all the vaccine formulation, the adjuvant, and the antibody are administered, for example, within 1 minute to 24 hours, preferably within 1 minute to 5 hours.

### EXAMPLES

Hereinafter, preferred specific aspects of the present invention will be described as Examples.

### [Example 1] Synthesis of Hyaluronic Acid (HA) Derivative

HA derivatives were synthesized by the method described in International Publication No. WO 2010/053140 or WO 2014/038641.

### [Example 2] Obtainment of Antigen

Antigenic peptides were purchased from Sigma-Genosys LP.

mERK2 p121
(amino acid sequence: NDHIAYFLYQILRGLQYIHS
ANVLHRDLKPSNLLLNT (SEQ ID NO: 1))

The sequence contained a 9-amino acid sequence (SEQ ID NO: 2: QYIHSANVL) from Q at position 16 to L at position 24 as a CD8-positive cytotoxic T cell recognition epitope sequence, and a 17-amino acid sequence (SEQ ID NO: 3: RGLQYIHSANVLHRDLK) from R at position 13 to K at position 29 as a CD4-positive helper T cell recognition epitope sequence.

MAGE-A4 p264
(amino acid sequence: GSNPARYEFLWGPRALAETS
YVKVLEHVVRVNARVRIAYP (SEQ ID NO: 4))

The sequence contained a 9-amino acid sequence (SEQ ID NO: 5: SNPARYEFL) from S at position 2 to L at position 10 as a CD8-positive cytotoxic T cell recognition epitope sequence and a 16-amino acid sequence (SEQ ID NO: 6: VKVLEHVVRVNARVRI) from V at position 22 to I at position 37 as a CD4-positive helper T cell recognition epitope sequence.

MAGE-A4 p265
(amino acid sequence: SNPARYEFL (SEQ ID NO: 7)).
MAGE-A4 p285
(amino acid sequence: VKVLEHVVRVNARVRIA
YP (SEQ ID NO: 8)).
TRP2TRP1gp100-6G
(amino acid sequence: SVYDFFVWLGGGGGGTWHRYHLLG
GGGGGEGSRNQDWL (SEQ ID NO: 9)).

The sequence contained TRP2 (amino acid sequence: SVYDFFVWL (SEQ ID NO: 13)), TRP1 (amino acid sequence: TWHRYHLL (SEQ ID NO: 14)), and gp100 (amino acid sequence: EGSRNQDWL (SEQ ID NO: 15)) given below as CD8-positive cytotoxic T cell recognition epitope sequences, and the epitopes were connected via 6 G.

TRP2TRP1gp100-6Y
(amino acid sequence: SVYDFFVWLYYYYYYTWHRYHLLY
YYYYYEGSRNQDWL (SEQ ID NO: 10)).

The sequence contained TRP2 (amino acid sequence: SVYDFFVWL (SEQ ID NO: 13)), TRP1 (amino acid sequence: TWHRYHLL (SEQ ID NO: 14)), and gp100 (amino acid sequence: EGSRNQDWL (SEQ ID NO: 15)) given below as CD8-positive cytotoxic T cell recognition epitope sequences, and the epitopes were connected via 6 Y.

TRP2TRP1gp100-4L
(amino acid sequence: SVYDFFVWLLLLLTWHRYHLLLLL
LLEGSRNQDWL (SEQ ID NO: 11)).

The sequence contained TRP2 (amino acid sequence: SVYDFFVWL (SEQ ID NO: 13)), TRP1 (amino acid sequence: TWHRYHLL (SEQ ID NO: 14)), and gp100 (amino acid sequence: EGSRNQDWL (SEQ ID NO: 15)) given below as CD8-positive cytotoxic T cell recognition epitope sequences, and the epitopes were connected via 4 L.

TRP2TRP1gp100-6L
(amino acid sequence: SVYDFFVWLLLLLLLTWHRYHLLL
LLLLLEGSRNQDWL (SEQ ID NO: 12)).

The sequence contained TRP2 (amino acid sequence: SVYDFFVWL (SEQ ID NO: 13)), TRP1 (amino acid sequence: TWHRYHLL (SEQ ID NO: 14)), and gp100 (amino acid sequence: EGSRNQDWL (SEQ ID NO: 15)) given below as CD8-positive cytotoxic T cell recognition epitope sequences, and the epitopes were connected via 6 L.

TRP2
(amino acid sequence: SVYDFFVWL (SEQ ID NO: 13)).
TRP1
(amino acid sequence: TWHRYHLL (SEQ ID NO: 14)).
gp100
(amino acid sequence: EGSRNQDWL (SEQ ID NO: 15)).
AH1gp70-6G
(amino acid sequence: LVQFIKDRISVVQAGGGGGGSPSYVYH
QF (SEQ ID NO: 16)).

The sequence contained gp70 (amino acid sequence: LVQFIKDRISVVQA (SEQ ID NO: 21)) given below as a CD4-positive helper T cell recognition epitope and AH1 (amino acid sequence: SPSYVYHQF (SEQ ID NO: 20)) given below as a CD8-positive cytotoxic T cell recognition epitope sequence, and the epitopes were connected via 6 G.

AH1gp70-6Y
(amino acid sequence: LVQFIKDRISVVQAYYYYYYSPSYVYH
QF (SEQ ID NO: 17)).

The sequence contained gp70 (amino acid sequence: LVQFIKDRISVVQA (SEQ ID NO: 21)) given below as a CD4-positive helper T cell recognition epitope and AH1 (amino acid sequence: SPSYVYHQF (SEQ ID NO: 20)) given below as a CD8-positive cytotoxic T cell recognition epitope sequence, and the epitopes were connected via 6 Y.

AH1gp70-4L
(amino acid sequence: LVQFIKDRISVVQALLLLSPSYVYH
QF (SEQ ID NO: 18)).

The sequence contained gp70 (amino acid sequence: LVQFIKDRISVVQA (SEQ ID NO: 21)) given below as a CD4-positive helper T cell recognition epitope and AH1 (amino acid sequence: SPSYVYHQF (SEQ ID NO: 20)) given below as a CD8-positive cytotoxic T cell recognition epitope sequence, and the epitopes were connected via 4 L.

AH1gp70-6L
(amino acid sequence: LVQFIKDRISVVQALLLLLLSPSYVYH
QF (SEQ ID NO: 19)).

The sequence contained gp70 (amino acid sequence: LVQFIKDRISVVQA (SEQ ID NO: 21)) given below as a CD4-positive helper T cell recognition epitope and AH1 (amino acid sequence: SPSYVYHQF (SEQ ID NO: 20)) given below as a CD8-positive cytotoxic T cell recognition epitope sequence, and the epitopes were connected via 6 L.

AH1
(amino acid sequence: SPSYVYHQF (SEQ ID NO: 20)).
gp70
(amino acid sequence: LVQFIKDRISVVQ
A (SEQ ID NO: 21)).
MAGE-A4 p264-4L
(amino acid sequence: GSNPARYEFLWGPRALLLLLYV
KVLEHVVRVNARVRIAYP (SEQ ID NO: 22)).

The sequence contained a 9-amino acid sequence (SEQ ID NO: 5: SNPARYEFL) from S at position 2 to L at position 10 as a CD8-positive cytotoxic T cell recognition epitope sequence and a 16-amino acid sequence (SEQ ID NO: 6: VKVLEHVVRVNARVRI) from V at position 22 to I at position 37 as a CD4-positive helper T cell recognition epitope sequence, and the epitopes were connected via 4 L.

MAGE-A4 p264-4W
(amino acid sequence: GSNPARYEFLWGPRALWWWWYVKVLEH
VVRVNARVRIAYP (SEQ ID NO: 23)).

The sequence contained a 9-amino acid sequence (SEQ ID NO: 5: SNPARYEFL) from S at position 2 to L at position 10 as a CD8-positive cytotoxic T cell recognition epitope sequence and a 16-amino acid sequence (SEQ ID NO: 6: VKVLEHVVRVNARVRI) from V at position 22 to I at position 37 as a CD4-positive helper T cell recognition epitope sequence, and the epitopes were connected via 4 W.

### [Example 3] Production of Complex of HA Derivative and Antigen

### (Example 3-1) Preparation of Complex of Antigen and HA Derivative

Each HA derivative (99k HA-C₆-Chol-42%, etc.) was dissolved at 12 mg/mL in ultrapure water, and ultrasonication (manufactured by Covaris Inc., E220X) was carried out. Each antigenic peptide was dissolved at 50 mg/mL in DMSO. The solution of the antigenic peptide in DMSO was added to the aqueous HA derivative solution, and the mixture was ultrasonicated for 30 minutes in a bath-type sonicator. The resultant was transferred to a dialysis kit (Slide-A-Lyzer, molecular weight cutoff: 3.5 K) and dialyzed against a 5 mM carbonate buffer solution (pH 9), a 10 mM phosphate buffer solution (pH 7), and a 10 mM phosphate buffer solution (pH 7) containing 10% sucrose in this order. The antigenic peptide concentration was quantified by reverse-phase chromatography analysis under conditions given below. The concentration was adjusted to 250 to 500 μg/mL to prepare an administration solution. If the concentration did not reach the intended one, the solution was concentrated with an ultrafiltration concentrator (Vivaspin 6, 5000 MwCO. Sartorius AG). The HA derivative was synthesized as described in Examples 1 and 2 of International Publication No. WO 2010/053140 A1. For example, 99k HA-C₆-Chol-42% shown in Table 1 of Example 4-1 denotes a HA derivative of the formula (I) wherein Z is a direct bond, R^{a} is a hydrogen atom, Y is C₆ alkylene, X¹ is —NR^{b}—COO—R, R^{b} is a hydrogen atom, and R is a cholesteryl group (i.e., cholesteryl 6-aminohexyl carbamate was introduced); the weight-average molecular weight is 99 kDa; and the rate of introduction of the cholesteryl group is 42%. Likewise, a HA derivative wherein the rate of introduction of the cholesteryl group is 41% is designated as 99k HA-C₆-Chol-41%, and a HA derivative wherein the rate of introduction of the cholesteryl group is 43% is designated as 99k HA-C₆-Chol-43%.

Reverse-Phase Chromatography Analysis Conditions

Analytical column: PLRP-S 1000 angstroms (Agilent Technologies, Inc.)

Column temperature: 40° C.

Mobile phase A: 0.1% aqueous TFA solution, mobile phase B: 0.10% TFA solution in acetonitrile

Flow rate: 2 mL/min

Detection: UV 254 nm

Injection volume: 50 μL

### [Comparative Example 1] Production of Complex of CHP and Antigen

A complex was prepared in accordance with International Publication No. WO 2015/050158. Cholesterol-modified pullulan (abbreviation: CHP) (CHP-80T; 1 to 2 cholesterol molecules were introduced per 100 monosaccharides) was obtained from NOF Corp. CHP was dissolved at a concentration of 10 mg/mL in phosphate-buffered saline (PBS) containing 6 M urea. A solution of an antigenic peptide in DMSO was added to the CHP solution, and the mixture was left overnight at room temperature in the dark. The mixed solution was transferred to a dialysis membrane (molecular weight cutoff: 3,500, Thermo Fisher Scientific Inc.) and dialyzed at 4° C. for 2 hours to overnight against PBS containing 0.6 M urea at a volume ratio of 100 times or more as an outer solution of the dialysis membrane. Subsequently, the resultant was dialyzed at 4° C. for 2 hours to overnight against PBS containing 0.06 M urea at a volume ratio of 100 times or more as an outer solution of the dialysis membrane. Again, the resultant was dialyzed at 4° C. for 2 hours to overnight against PBS at a volume ratio of 100 times or more as an outer solution of the dialysis membrane. After recovery of an inner solution of the dialysis membrane, the antigen concentration was quantified by the method described in Example 3-1. The concentration was adjusted to prepare an administration solution. If the concentration did not reach the intended one, the solution was concentrated with an ultrafiltration concentrator (Vivaspin 6, 5000 MwCO, Sartorius AG).

### [Example 4] Antigen-Specific T Cell Induction Test

Experimental Method

The complex of the antigenic peptide and the HA derivative prepared in Example 3-1 or the complex of CHP and the antigenic peptide prepared in Comparative Example 1 was subcutaneously administered in an amount corresponding to 0.05 to 0.1 mg of the antigen to the right back of each BALB/c mouse (female, Japan SLC, Inc.; body weight: 15 to 25 g) or C57BL/6 mouse (female, Japan SLC, Inc.: body weight: 15 to 25 g). In the case of administering an adjuvant (CpG oligo DNA (ODN1668, manufactured by InvivoGen), 0.05 mg), the adjuvant was subsequently administered subcutaneously near the administration site. The number of doses is 2, and the interval between the doses was 1 week. 1 week after final administration, spleen cells were separated from the recipient mouse by the following procedures: the spleen was isolated from the mouse and washed with RPMI1640 medium containing 10% fetal bovine serum (FBS), and blood was removed. The spleen was ground, and liberated cells were then recovered into RPMI1640 medium containing FBS. After centrifugation (300×g, 5 min, 4° C.), the supernatant was removed, and 1 to 2 mL of an ACK solution (manufactured by Sigma-Aldrich Co. LLC) was added to the cells. 1 minute later, 30 mL of RPMI1640 medium containing FBS was added thereto, and the mixture was centrifuged (300×g, 5 min, 4° C.). The supernatant was removed, and 10 mL of RPMI1640 medium containing FBS was added to the cells, followed by filter treatment using a40 μm cell strainer and centrifugation (300×g, 5 min, 4° C.). The supernatant was removed, and 20 mL of RPMI1640 medium containing FBS was added to the cells, followed by centrifugation (300×g, 5 min, 4° C.). The supernatant was removed, and the cells were suspended in an appropriate amount of RPMI1640 medium containing FBS. The number of cells was counted, and the cells were then suspended at a cell density of 2×10⁷ cells/mL in RPMI1640 medium containing FBS.

The mouse spleen cells were added at 5×10⁶ cells/0.4 mL per well to a 24-well culture plate (Nunc). 50 μL of 100 μg/mL peptide for T cell stimulation was added to each well, and the cells were cultured at 37° C. for 30 minutes under 5% CO₂. Then, GolgiPlug (BD Biosciences) diluted 100-fold with RPMI1640 medium containing FBS was added at 50 μL per well, and the cells were cultured at 37° C. for 5 hours under 5% CO₂. The cells were recovered and transferred to a 96-well V-bottom microplate (Nunc). A supernatant was removed by centrifugation (2000 rpm, 2 min, 4° C.; the same holds true for the conditions below). Then, the cells were suspended in 200 μL of a buffer for staining (PBS containing 0.5% bovine serum albumin (BSA)) per well. A supernatant was removed by centrifugation. Then, 50 μL of a solution containing a fluorescently labeled anti-CD8 antibody (manufactured by Invitrogen Corp.) and a fluorescently labeled anti-CD4 antibody (BD Biosciences) was added to each well and mixed, and the plate was then left standing at 4° C. for 15 minutes in the dark. 150 μL of a buffer for staining was added to each well. A supernatant was removed by centrifugation, and the cells were then further washed with 200 μL of a buffer for staining. 100 μL of Cytofix/Cytoperm buffer (BD Biosciences) was added to each well and gently mixed. The plate was left standing at room temperature for 20 minutes in the dark, and the cells were then further washed twice with a buffer for staining. 200 μL of a buffer for staining was added to each well and refrigerated overnight with light shielded. A supernatant was removed by centrifugation. Then, 200 μL of Perm/Wash buffer (BD Biosciences) was added to each well, and the plate was left standing at room temperature for 3 minutes with light shielded. A supernatant was removed by centrifugation. Then, 50 μL of Perm/Wash buffer supplemented with each anti-cytokine antibody was added to the cells for gentle suspension, and the plate was then left standing at room temperature for 15 minutes in the dark. The cells were washed with 150 μL of Penn/Wash buffer, then resuspended in 200 μL of a buffer for staining, and transferred to a round-bottom polystyrene tube (BD Biosciences). The cells were analyzed in a flow cytometer (FACS Canto II, BD Biosciences) using attached analytical software (FACSDiva).

### (Example 4-1) T Cell Induction Test Using mERK2 Peptide

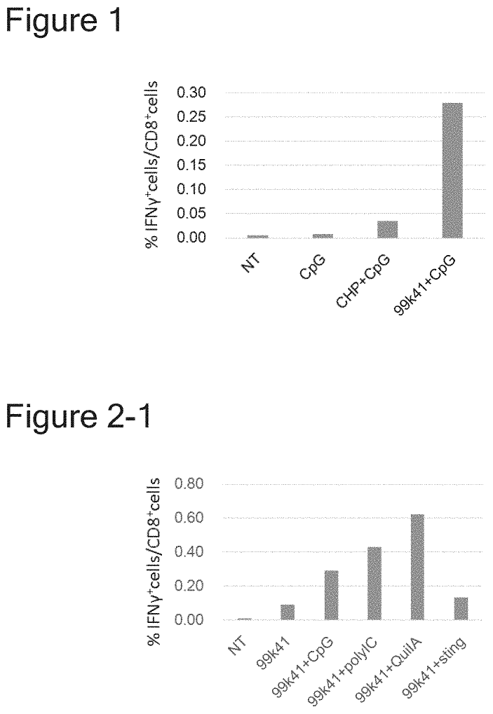

The test was conducted according to the method described in Example 4 by using BALB/c mice and administering each sample of Table 1 twice. The adjuvant used was CpG oligo DNA (ODN1668, manufactured by InvivoGen). FIG. 1 shows the ratio of the number of interferon gamma-producing CD8⁺ cells to the number of all CD8⁺ cells when mERK2 p121 was used as a peptide for CD8⁺ T cell stimulation.

TABLE 1
Administration sample

| | | Peptide | Adjuvant |
|---|---|---|---|
| | | species, | species, |
| Abbreviation | Carrier | Dose | Dose |
| CpG | None | mERK2 p121 | CpG |
| | | 0.1 mg | 0.05 mg |
| CHP + CpG | CHP-80T; 80 k | mERK2 p121 | CpG |
| | | 0.1 mg | 0.05 mg |
| 99k41 + CpG | 99 k HA-C₆-Chol-41% | mERK2 p121 | CpG |
| | | 0.1 mg | 0.05 mg |

Use of the HA derivative as a carrier was confirmed to be able to induce T cells at a high level.

### (Example 4-2) T Cell Induction Test Using mERK2 Peptide (Adjuvant Effect—1)

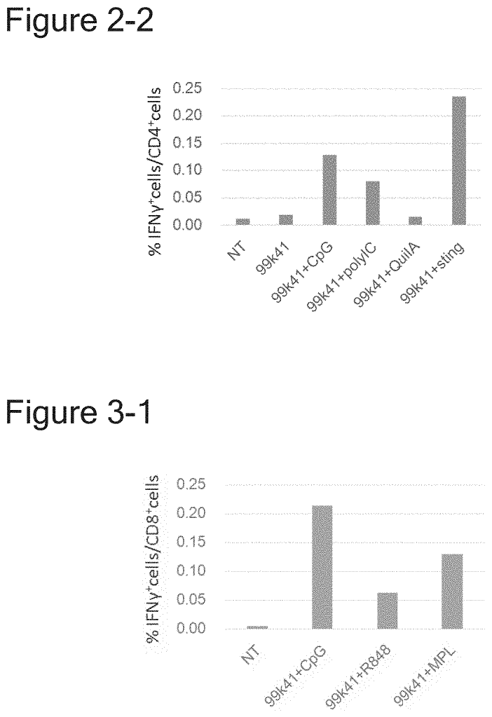

The test was conducted according to the method described in Example 4 by using BALB/c mice and administering each sample of Table 2 twice. The adjuvant used was CpG oligo DNA (ODN1668, manufactured by InvivoGen), polyIC (HMW VacciGrade, manufactured by InvivoGen), QuilA (manufactured by InvivoGen), or a Sting agonist (2′3′-cGAM(PS)2(Rp/Sp), manufactured by InvivoGen). FIG. 2-1 shows the ratio of the number of interferon gamma-producing CD8⁺ cells to the number of all CD8⁺ cells when mERK2 p121 was used as a peptide for CD8⁺ T cell stimulation. FIG. 2-2 shows the ratio of the number of interferon gamma-producing CD4⁺ cells to the number of all CD4⁺ cells when mERK2 p121 was used as a peptide for CD4⁺ T cell stimulation.

TABLE 2
Administration sample

| | | Peptide | Adjuvant |
|---|---|---|---|
| | | species, | species, |
| Abbreviation | Carrier | Dose | Dose |
| 99k41 | 99 k HA-C₆-Chol-41% | mERK2 p121 | None |
| | | 0.1 mg | |
| 99k41 + CpG | 99 k HA-C₆-Chol-41% | mERK2 p121 | CpG |
| | | 0.1 mg | 0.05 mg |
| 99k41 + PolyIC | 99 k HA-C₆-Chol-41% | mERK2 p121 | PolyIC |
| | | 0.1 mg | 0.05 mg |
| 99k41 + QuilA | 99 k HA-C₆-Chol-41% | mERK2 p121 | QuilA |
| | | 0.1 mg | 0.05 mg |
| 99k41 + Sting | 99 k HA-C₆-Chol-41% | mERK2 p121 | Sting |
| | | 0.1 mg | 0.05 mg |

Use of any of the adjuvants was confirmed to be able to induce T cells.

### (Example 4-3) T Cell Induction Test Using mERK2 Peptide (Adjuvant Effect—2)

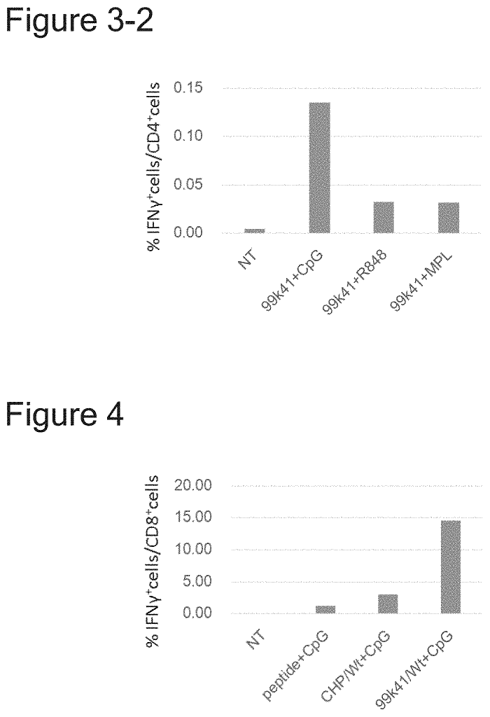

The test was conducted according to the method described in Example 4 by using BALB/c mice and administering each sample of Table 3 twice. The adjuvant used was CpG oligo DNA (ODN1668, manufactured by InvivoGen), R848 (VacciGrade, VacciGrade, manufactured by InvivoGen), or MPL (MPLAs VacciGrade, manufactured by InvivoGen). FIG. 3-1 shows the ratio of the number of interferon gamma-producing CD8⁺ cells to the number of all CD8⁺ cells when mERK2 p121 was used as a peptide for CD8⁺ T cell stimulation. FIG. 3-2 shows the ratio of the number of interferon gamma-producing CD4⁺ cells to the number of all CD4⁺ cells when mERK2 p121 was used as a peptide for CD4⁺ T cell stimulation.

TABLE 3
Administration sample

| | | Peptide | Adjuvant |
|---|---|---|---|
| | | species, | species, |
| Abbreviation | Carrier | Dose | Dose |
| 99k41 + CpG | 99 k HA-C₆-Chol-41% | mERK2 p121 | CpG |
| | | 0.1 mg | 0.05 mg |
| 99k41 + R848 | 99 k HA-C₆-Chol-41% | mERK2 121p | R848 |
| | | 0.1 mg | 0.05 mg |
| 99k41 + MPL | 99 k HA-C₆-Chol-41% | mERK2 121p | MPL |
| | | 0.1 mg | 0.05 mg |

Use of any of the adjuvants was confirmed to be able to induce T cells.

### (Example 4-4) T Cell Induction Test Using MAGE-A4 p264 Peptide

The test was conducted according to the method described in Example 4 by using BALB/c mice and administering each sample of Table 4 twice. FIG. 4 shows the ratio of the number of interferon gamma-producing CD8⁺ cells to the number of all CD8⁺ cells when MAGE-A4 p265 was used as a peptide for CD8⁺ T cell stimulation.

[Table 4]

TABLE 4
Administration sample

| | | Peptide | Adjuvant |
|---|---|---|---|
| | | species, | species, |
| Abbreviation | Carrier | Dose | Dose |
| peptide + CpG | None | MAGE-A4 p264 | CpG |
| | | 0.075 mg | 0.05 mg |
| CHP/Wt + CpG | CHP-80T; 80 k | MAGE-A4 p264 | CpG |
| | | 0.075 mg | 0.05 mg |
| 99k41/Wt + CpG | 99 k HA-C₆-Chol-41% | MAGE-A4 p264 | CpG |
| | | 0.075 mg | 0.05 mg |

Use of the HA derivative was confirmed to be able to induce T cells at a high level.

### (Example 4-5) T Cell Induction Test Using MAGE-A4 p264 Peptide (without Adjuvant)

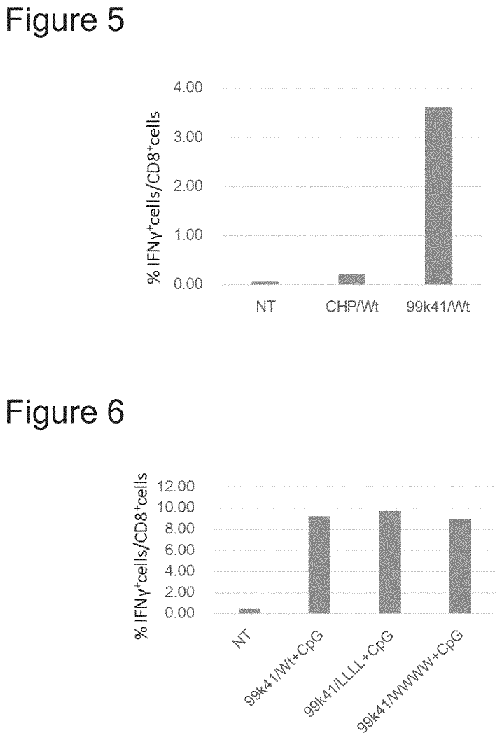

The test was conducted according to the method described in Example 4 by using BALB/c mice and administering each sample of Table 5 twice. FIG. 5 shows the ratio of the number of interferon gamma-producing CD8⁺ cells to the number of all CD8⁺ cells when MAGE-A4 p265 was used as a peptide for CD8⁺ T cell stimulation.

TABLE 5
Administration sample

| | | Peptide | Adjuvant |
|---|---|---|---|
| | | species, | species, |
| Abbreviation | Carrier | Dose | Dose |
| CHP/Wt | CHP-80T; 80 k | MAGE-A4 p264 | None |
| | | 0.075 mg | |
| 99k41/Wt | 99 k HA-C₆-Chol-41% | MAGE-A4 p264 | None |
| | | 0.075 mg | |

Use of the HA derivative as a carrier was confirmed to be able to induce T cells at a high level even when no adjuvant was used.

### (Example 4-6) T Cell Induction Test Using MAGE-A4 p264 Peptide

The test was conducted according to the method described in Example 4 by using BALB/c mice and administering each sample of Table 6 twice. FIG. 6 shows the ratio of the number of interferon gamma-producing CD8⁺ cells to the number of all CD8⁺ cells when MAGE-A4 p265 was used as a peptide for CD8⁺ T cell stimulation.

TABLE 6
Administration sample

| | | Peptide species, | Adjuvant species, |
|---|---|---|---|
| Abbreviation | Carrier | Dose | Dose |
| 99k41/Wt + CpG | 99k HA-C₆-Chol-41% | MAGE-A4 p264 | CpG |
| | | 0.075 mg | 0.05 mg |
| 99k412/LLLL + CpG | 99k HA-C₆-Chol-41% | MAGE-A4 p264-4L | CpG |
| | | 0.075 mg | 0.05 mg |
| 99k41lWWWW + CpG | 99k HA-C₆-Chol-41% | MAGE-A4 p264-4W | CpG |
| | | 0.075 mg | 0.05 mg |

Use of the peptide containing any of the amino acid linkers was found to be able to induce T cells at a high level.

### (Example 4-7) T Cell Induction Test Using TRP2TRP1gp100 Peptide

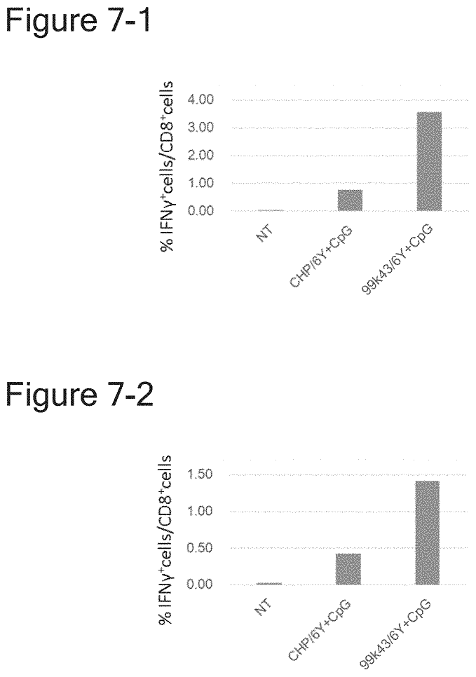

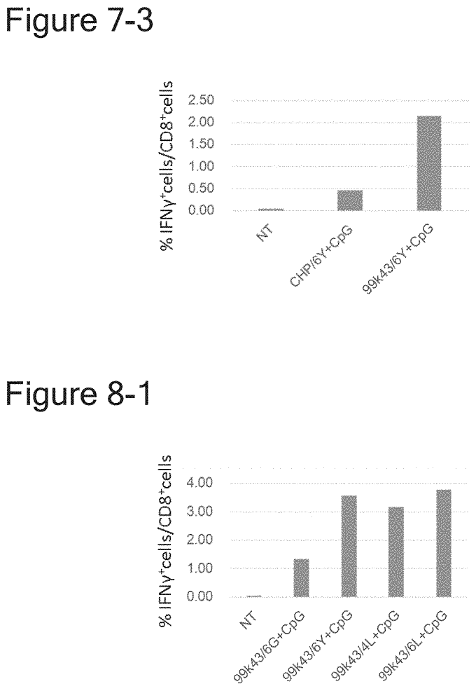

The test was conducted according to the method described in Example 4 by using C57BL/6 mice and administering each sample of Table 7 twice. FIGS. 7-1, 7-2, and 7-3 show the ratio of the number of interferon gamma-producing CD8⁺ cells to the number of all CD8⁺ cells when TRP1, TRP2, and gp100, respectively, were used as peptides for CD8⁺ T cell stimulation.

TABLE 7
Administration sample

| Abbreviation | Carrier | Peptide species, Dose | Adjuvant species, Dose |
|---|---|---|---|
| CHP/6Y + CpG | CHP-80T; 80 k | TRP2TRP1gp100-6Y | CpG |
| | | 0.1 mg | 0.05 mg |
| 99k43/6Y + CpG | 99 k HA-C₆-Chol-43% | TRP2TRP1gp100-6Y | CpG |
| | | 0.1 mg | 0.05 mg |

Use of the HA derivative as a carrier was confirmed to be able to induce T cells at a high level for each of TRP1, TRP2, and gp100.

### (Example 4-8) T Cell Induction Test Using TRP2TRP1gp100 Peptide

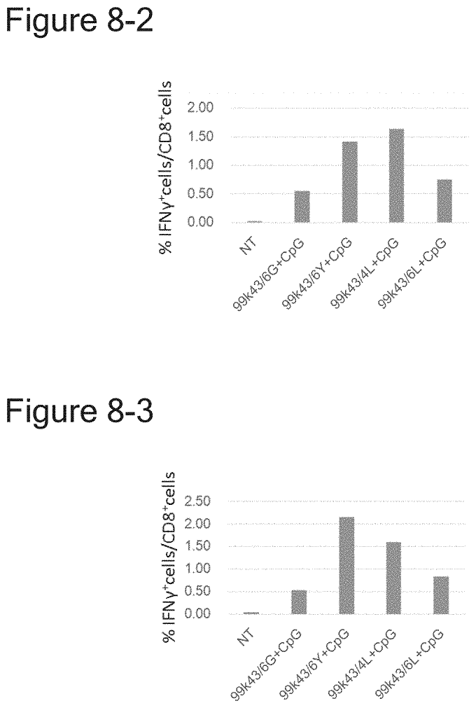

The test was conducted according to the method described in Example 4 by using C57BL/6 mice and administering each sample of Table 8 twice. FIGS. 8-1, 8-2, and 8-3 show the ratio of the number of interferon gamma-producing CD8⁺ cells to the number of all CD8⁺ cells when TRP1, TRP2, and gp100, respectively, were used as peptides for CD8⁺ T cell stimulation.

TABLE 8
Administration sample

| Abbreviation | Carrier | Peptide species, Dose | Adjuvant species, Dose |
|---|---|---|---|
| 99k43/6G + CpG | 99 k HA-C₆-Chol-43% | TRP2FRP1gp100-6G | CpG |
| | | 0.1 mg | 0.05 mg |
| 99k43/6Y + CpG | 99 k HA-C₆-Chol-43% | TRP2TRP1gp100-6Y | CpG |
| | | 0.1 mg | 0.05 mg |
| 99k43/4L + CpG | 99 k HA-C₆-Chol-43% | TRP2TRP1gp100-4L | CpG |
| | | 0.1 mg | 0.05 mg |
| 99k43/6L + CpG | 99 k HA-C₆-Chol-43% | TRP2TRP gp100-6L | CpG |
| | | 0.1 mg | 0.05 mg |

Use of any peptide linker was found to be able to induce T cells at a high level.

### (Example 4-9) T Cell Induction Test Using AH1gp70 Peptide

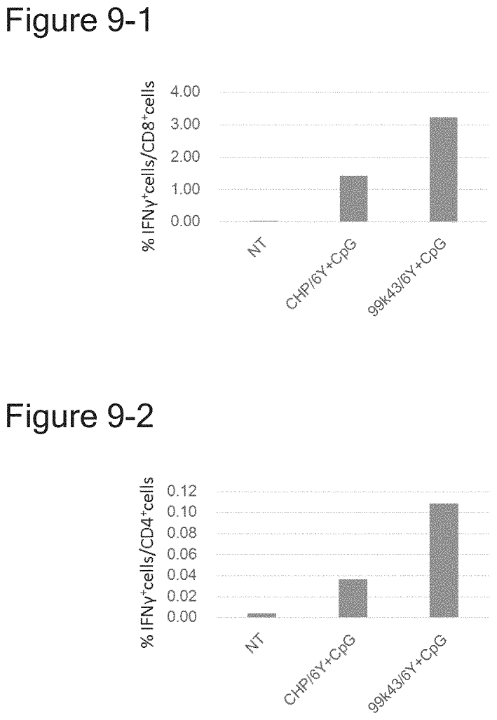

The test was conducted according to the method described in Example 4 by using BALB/c mice and administering each sample of Table 9 twice. FIG. 9-1 shows the ratio of the number of interferon gamma-producing CD8⁺ cells to the number of all CD8⁺ cells when AH1 was used as a peptide for CD8⁺ T cell stimulation. FIG. 9-2 shows the ratio of the number of interferon gamma-producing CD4⁺ cells to the number of all CD4⁺ cells when gp70 was used as a peptide for CD4⁺ T cell stimulation.

TABLE 9
Administration sample

| | | Peptide | Adjuvant |
|---|---|---|---|
| | | species, | species, |
| Abbreviation | Carrier | Dose | Dose |
| CHP/6Y + CpG | CHP-80T; 80 k | AH1gp70-6Y | CpG |
| | | 0.1 mg | 0.05 mg |
| 99k43/6Y + CpG | 99 k HA-C₆-Chol-43% | AH1gp70-6Y | CpG |
| | | 0.1 mg | 0.05 mg |

Use of the HA derivative was confirmed to be able to induce T cells at a high level for each of AH1 and gp70.

### (Example 4-10) T Cell Induction Test Using AH1gp70 Peptide

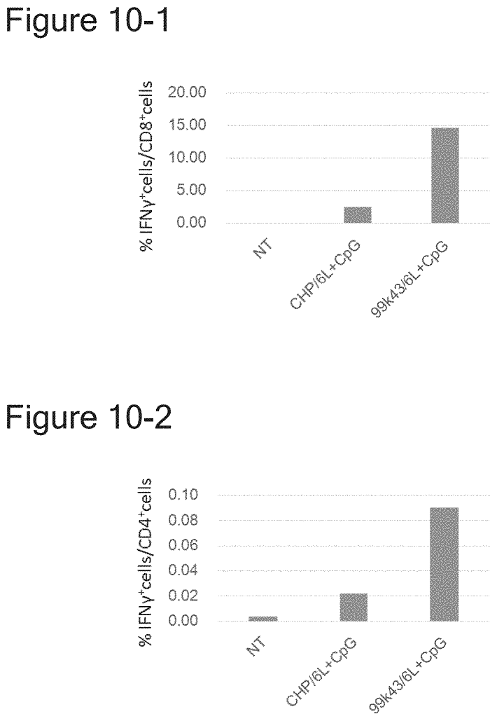

The test was conducted according to the method described in Example 4 by using BALB/c mice and administering each sample of Table 10 twice. FIG. 10-1 shows the ratio of the number of interferon gamma-producing CD8⁺ cells to the number of all CD8⁺ cells when AH1 was used as a peptide for CD8⁺ T cell stimulation. FIG. 10-2 shows the ratio of the number of interferon gamma-producing CD4⁺ cells to the number of all CD4⁺ cells when gp70 was used as a peptide for CD4⁺ T cell stimulation.

TABLE 10
Administration sample

| | | Peptide | Adjuvant |
|---|---|---|---|
| Abbreviation | Carrier | species, Dose | species, Dose |
| CHP/6L + | CHP-80T; 80k | AH1gp70-6L | CpG |
| CpG | | 0.1 mg | 0.05 mg |
| 99k43/6L + | 99k HA-C₆-Chol-43% | AH1gp70-6L | CpG |
| CpG | | 0.1 mg | 0.05 mg |

Use of the HA derivative was confirmed to be able to induce T cells at a high level for each of AH1 and gp70.

### (Example 4-11) T Cell Induction Test Using AH1gp70 Peptide

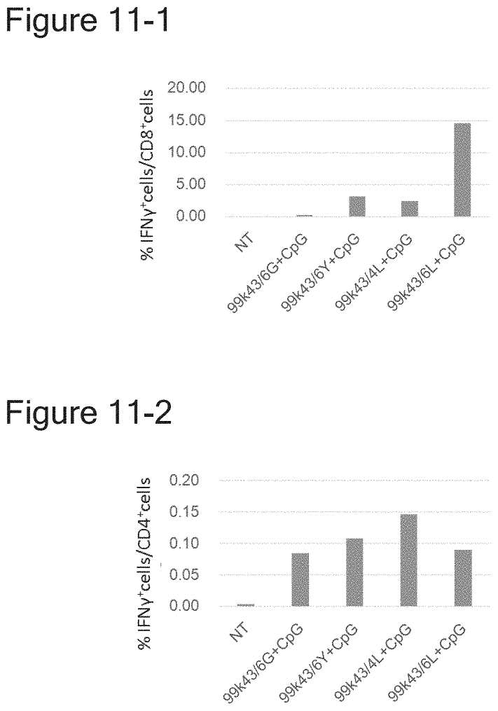

The test was conducted according to the method described in Example 4 by using BALB/c mice and administering each sample of Table 11 twice. FIG. 11-1 shows the ratio of the number of interferon gamma-producing CD8⁺ cells to the number of all CD8⁺ cells when AH1 was used as a peptide for CD8⁺ T cell stimulation. FIG. 11-2 shows the ratio of the number of interferon gamma-producing CD4⁺ cells to the number of all CD4⁺ cells when gp70 was used as a peptide for CD4⁺ T cell stimulation.

TABLE 11
Administration sample

| | | Peptide | Adjuvant |
|---|---|---|---|
| Abbreviation | Carrier | species, Dose | species, Dose |
| 99k43/6G + | 99k HA-C₆-Chol-43% | AH1gp70-6G | CpG |
| CpG | | 0.1 mg | 0.05 mg |
| 99k43/6Y + | 99k HA-C₆-Chol-43% | AH1gp70-6Y | CpG |
| CpG | | 0.1 mg | 0.05 mg |
| 99k43/4L + | 99k HA-C₆-Chol-43% | AH1gp70-4L | CpG |
| CpG | | 0.1 mg | 0.05 mg |
| 99k43/6L + | 99k HA-C₆-Chol-43% | AH1gp70-6L | CpG |
| CpG | | 0.1 mg | 0.05 mg |

Use of the peptide species containing any of the amino acid linkers was found to be able to induce T cells at a high level.

### [Example 5] Mouse Tumor Growth Test

Experimental Method

A mouse fibrosarcoma CMS5a cell line expressing mERK2, a mouse colorectal cancer cell CT26 cell line expressing AH1 and gp70, or a mouse melanoma B16F10 cell line expressing TRP2, TRP1, and gp100 was subcutaneously transplanted. The CMS5a cell line and the CT26 cell line were each subcutaneously transplanted in an amount of 1×10⁶ cultured cells/100 μL/individual to each BALB/c mouse (female: body weight: 15 to 25 g). The B16F10 cell line was subcutaneously transplanted in an amount of 2×10′ cultured cells/100 μL/individual to each C57BL/6 mouse (female; body weight: 15 to 25 g). Then, tumor volumes were measured over time.

### (Comparative Example 5-1) Preparation of Emulsion Formulation

An emulsion was produced from an antigenic peptide, 0.5 mL of a Freund's incomplete adjuvant (manufactured by InvivoGen), and 0.5 mL of saline using two hard glass syringes with lock fittings connected through a syringe connector. The antigenic peptide concentration was set to 0.5 mg/mL.

### (Example 5-1) Mouse Fibrosarcoma CMS5a

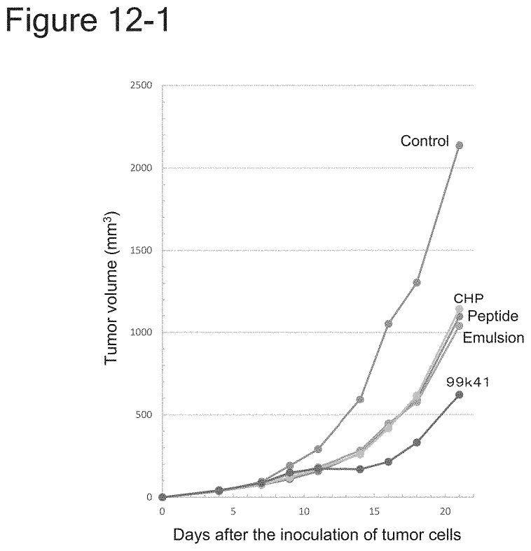

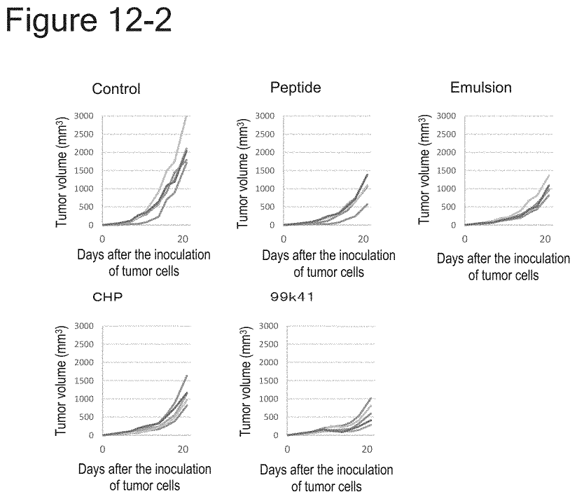

Each sample of Table 12 was subcutaneously administered with the frequency described in Table 12 to the mouse fibrosarcoma CMS5a-bearing mice prepared by the method described in Example 5 to conduct a mouse tumor growth test. FIG. 12-1 shows changes in average tumor volume, and FIG. 12-2 shows changes in each individual.

TABLE 12
Administration sample

| | | Peptide | Adjuvant | Administration |
|---|---|---|---|---|
| | | species, | species, | day (after |
| Abbreviation | Carrier | Dose | Dose | transplantation, |
| Peptide | None | mERK2 p121 | CpG | Day 4, day 11 |
| | | 0.05 mg | 0.05 mg | |
| Emulsion | Emulsion | mERK2 p121 | CpG | Day 4, day 11 |
| | | 0.05 mg | 0.05 mg | |
| CHP | CHP-80T; 80k | mERK2 p121 | CpG | Day 4; day 11 |
| | | 0.05 mg | 0.05 mg | |
| 99k41 | 99k HA-C₆- | mERK2 p121 | CpG | Day 4, day 11 |
| | Chol-41% | 0.05 mg | 0.05 mg | |

The HA derivative (99k41) group was shown to have a high tumor growth suppressive effect as compared with the peptide group, the emulsion group, and the CHP group.

### (Example 5-2) Mouse Fibrosarcoma CMS5a

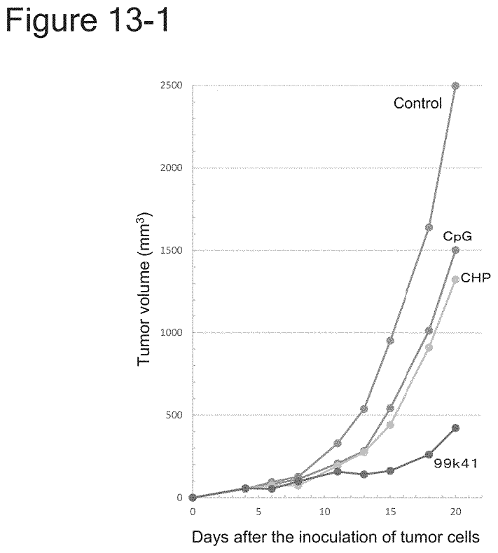

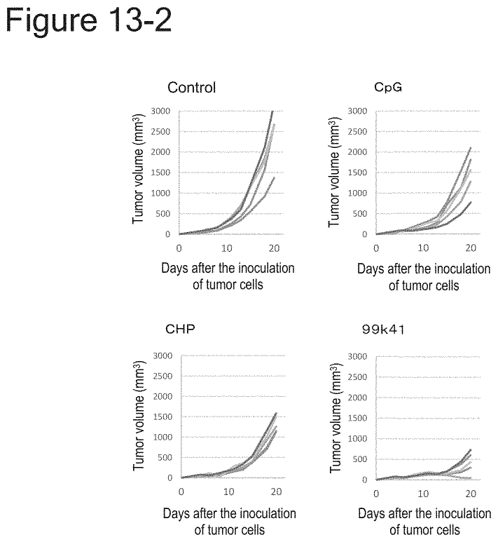

Each sample of Table 13 was subcutaneously administered with the frequency described in Table 13 to the mouse fibrosarcoma CMS5a-bearing mice prepared by the method described in Example 5 to conduct a mouse tumor growth test. FIG. 13-1 shows changes in average tumor volume, and FIG. 13-2 shows changes in each individual.

TABLE 13
Administration sample

| | | Peptide | Adjuvant | Administration |
|---|---|---|---|---|
| | | species | species, | day (after |
| Abbreviation | Carrier | Dose | Dose | transplantation |
| CpG | None | None | CpG | Day 4, day 11 |
| | | | 0.05 mg | |
| CHP | CHP-80T; | mERK2 p121 | CpG | Day 4, day 11 |
| | 80k | 0.05 mg | 0.05 mg | |
| 99k41 | 99k HA-C₆- | mERK2 p121 | CpG | Day 4, day 11 |
| | Chol-41% | 0.05 mg | 0.05 mg | |

The HA derivative (99k41) group was shown to have a high tumor growth suppressive effect as compared with the CpG-alone group and the CHP group.

### (Example 5-3) Mouse Fibrosarcoma CMS5a

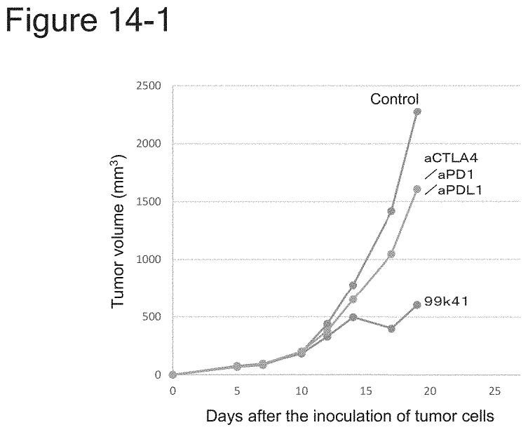

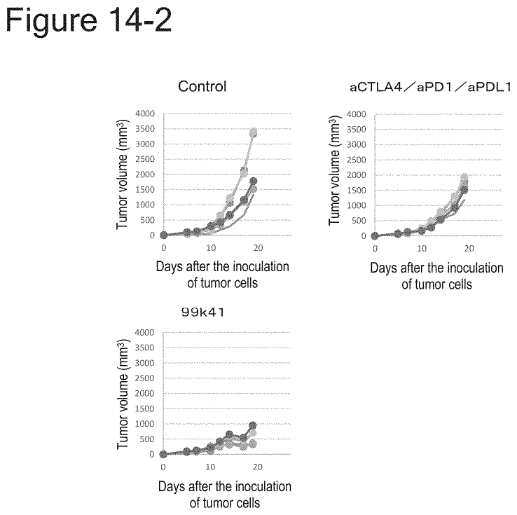

Each sample of Table 14 was subcutaneously administered with the frequency described in Table 14 to the mouse fibrosarcoma CMS5a-bearing mice prepared by the method described in Example 5 to conduct a mouse tumor growth test. The antibodies were intraperitoneally administered. FIG. 14-1 shows changes in average tumor volume, and FIG. 14-2 shows changes in each individual. The anti-CTLA4 antibody, the anti-PD1 antibody, and the anti-PDL1 antibody were each obtained from Bio X Cell.

TABLE 14
Admistration sample

| | | | | | Admin- |
|---|---|---|---|---|---|
| | | | | | istration |
| | | Peptide | Adjuvant | Antibody | day (after |
| Abbre- | | species, | species, | species | trans- |
| viation | Carrier | Dose | Dose | Dose | plantation) |
| aCTLA4/ | None | None | None | Anti- | Day 7, |
| aPD1/ | | | | CTLA4 | day 11, |
| aPDL1 | | | | antibody | day 14, |
| | | | | (0.2 mg) + | day 18 |
| | | | | Anti-PD1 | |
| | | | | antibody | |
| | | | | (0.2 mg) + | |
| | | | | Anti-PDL1 | |
| | | | | antibody | |
| | | | | (0.2 mg) | |
| 99k41 | 99k HA- | mERK2 | CpG | None | Day 7, |
| | C₆- | p121 | 0.05 mg | | day 13 |
| | Chol-41% | 0.05 mg | | | |

The HA derivative (99k41) group exhibited a high tumor growth suppressive effect even by administration from day 7, and the effect was shown to be higher than that of the antibodies.

### (Example 5-4) Mouse Fibrosarcoma CMS5a

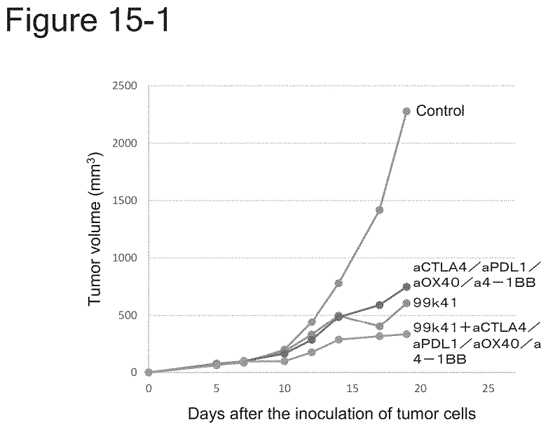

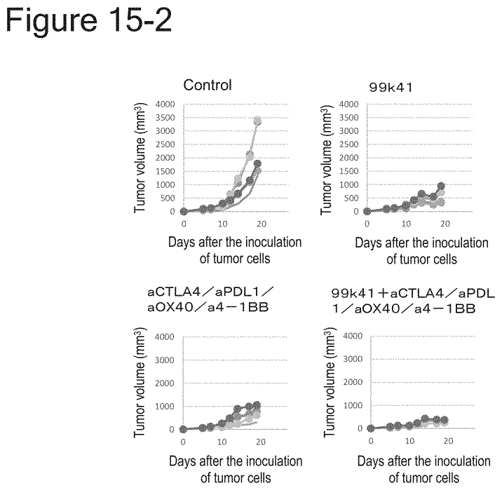

Each sample of Table 15 was subcutaneously administered with the frequency described in Table 15 to the mouse fibrosarcoma CMS5a-bearing mice prepared by the method described in Example 5 to conduct a mouse tumor growth test. The antibodies were intraperitoneally administered. FIG. 15-1 shows changes in average tumor volume, and FIG. 15-2 shows changes in each individual. The anti-CTLA4 antibody, the anti-PDL1 antibody, the anti-OX40 antibody, and the anti-4-1BB antibody were each obtained from Bio X Cell.

TABLE 15
Administration sample

| | | | | | Admin- |
|---|---|---|---|---|---|
| | | | | | istration |
| | | Peptide | Adjuvant | Antibody | day (after |
| Abbre- | | species, | species, | species | trans- |
| viation | Carrier | Dose | Dose | Dose | plantation) |
| aCTLA4/ | None | None | None | Anti-CTLA4 | Day 7, |
| aPDL1/ | | | | antibody (0.2 | day 11 |
| aOX40/ | | | | mg) + Anti- | day 14, |
| a4-1BB | | | | PDL1 antibody | day 18 |
| | | | | (0.2 mg) + Anti- | |
| | | | | OX40 antibody | |
| | | | | (0.2 mg) + | |
| | | | | Anti-4-1BB | |
| | | | | antibody | |
| | | | | (0.2 mg) | |
| 99k41 | 99k | mERK2 | CpG | None | Day 7, |
| | HA- | p121 | 0.05 mg | | day 13 |
| | C₆- | 0.05 mg | | | |
| | Chol- | | | | |
| | 41% | | | | |
| 99k41 + | 99k | mERK2 | CpG | Anti-CTLA4 | 99k42: |
| aCTLA4/ | HA- | p121 | 0.05 mg | antibody | day 7, |
| aPDL1/ | C₆- | 0.05 mg | | (0.2 mg) + Anti- | day 13, |
| aOX40/ | Chol- | | | PDL1 antibody | antibody: |
| a4-1BB | 41% | | | (0.2 mg) + Anti- | day 7, |
| | | | | OX40 antibody | day 11, |
| | | | | (0.2 mg) + Anti- | day 14, |
| | | | | 4-1BB | day 18 |
| | | | | antibody | |
| | | | | (0.2 mg) | |

The HA derivative (99k41) was shown to have a higher tumor growth suppressive effect based on the mERK2 peptide by combination with the antibodies (aCTLA4/aPDL1/aOX40/a4-1BB).

### (Example 5-5) Mouse Colorectal Cancer Cell CT26

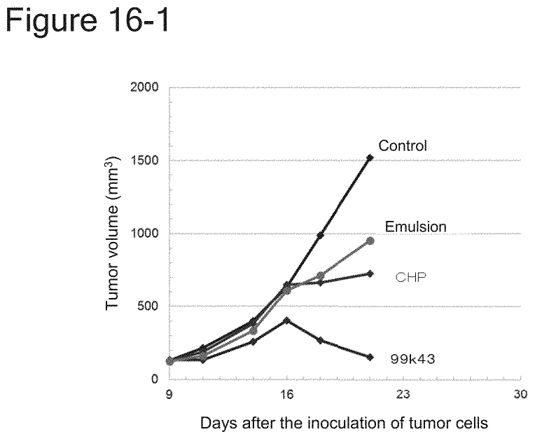

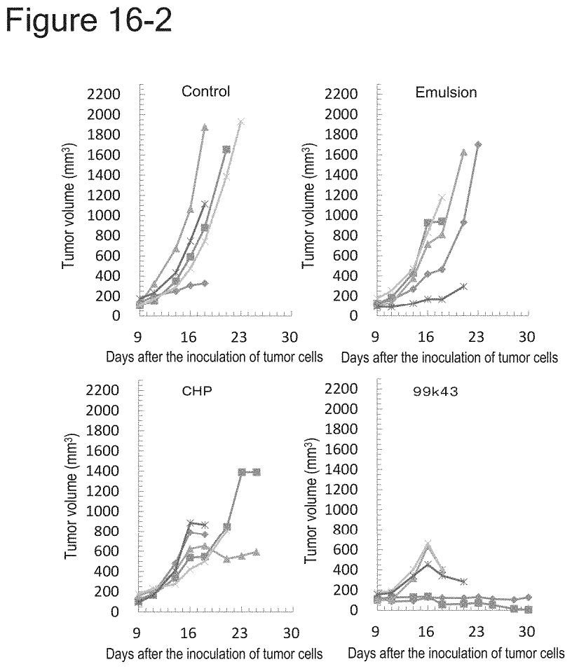

Each sample of Table 16 was subcutaneously administered with the frequency described in Table 16 to the mouse colorectal cancer cell CT26-bearing mice prepared by the method described in Example 5 to conduct a mouse tumor growth test. FIG. 16-1 shows changes in average tumor volume, and FIG. 16-2 shows changes in each individual.

TABLE 16
Administration sample

| | | Peptide | Adjuvant | Administration |
|---|---|---|---|---|
| | | species, | species, | day after |
| Abbreviation | Carrier | Dose | Dose | transplantation) |
| Emulsion | Emulsion | AH1gp70-6L | CpG | Day 9, day 16, |
| | | 0.05 mg | 0.05 mg | day 23 |
| CHP | CHP-80T; | AH1gp70-6L | CpG | Day 9, day 16, |
| | 80k | 0.05 mg | 0.05 mg | day 23 |
| 99k43 | 99k HA-C₆- | AH1gp70-6L | CpG | Day 9, day 16, |
| | Chol-43% | 0.05 mg | 0.05 mg | day 23 |

The HA derivative (99k43) group was shown to have a high tumor growth suppressive effect as compared with the emulsion group and the CHP group.

### (Example 5-6) Mouse Melanoma B16F10

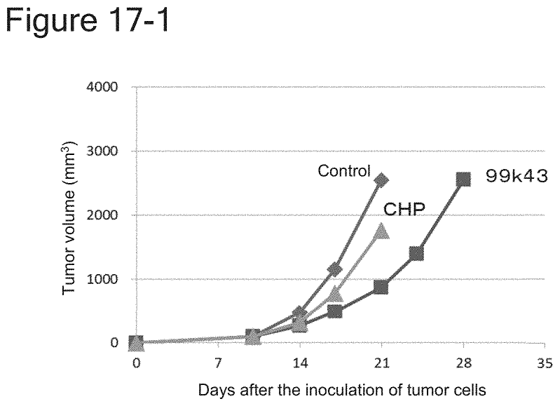

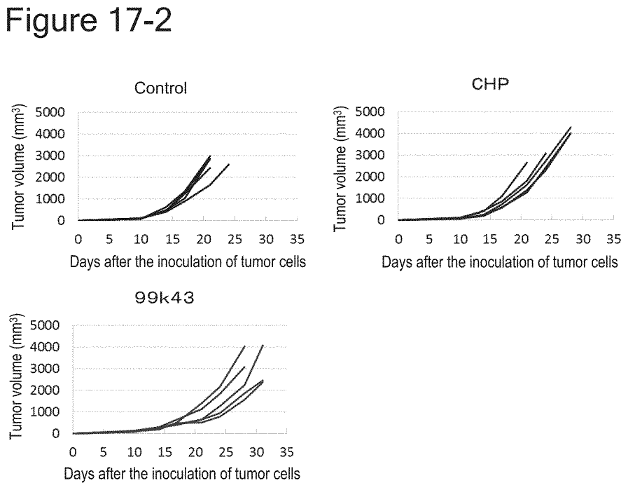

Each sample of Table 17 was subcutaneously administered with the frequency described in Table 17 to the mouse melanoma B16F10-bearing mice prepared by the method described in Example 5 to conduct a mouse tumor growth test. FIG. 17-1 shows changes in average tumor volume, and FIG. 17-2 shows changes in each individual.

TABLE 17
Administration sample

| | | Peptide | Adjuvant | Administration |
|---|---|---|---|---|
| Abbre- | | species, | species, | day (after |
| viation | Carrier | Dose | Dose | transplantation) |
| CHP | CHP-80T; | TRP2TRP1gp100- | CpG | Day 10, day |
| | 80k | 6Y 0.05 mg | 0.05 mg | 13, day 17 |
| 99k43 | 99k HA-C₆- | TRP2TRP1gp100- | CpG | Day 10, day |
| | Chol-43% | 6Y 0.05 mg | 0.05 mg | 13, day 17 |

The HA derivative (99k43) group was shown to have a high tumor growth suppressive effect as compared with the CHP group.

### [Example 6] Lymph Node Delivery

Experimental Method

Each sample prepared by the method described in Example 3 was subcutaneously administered to the right back of each BALB/c mouse (body weight: 15 to 25 g) using fluorescently labeled mERK2 p121 (fluorescein-conjugated) 24 hours later and 72 hours later, the right inguinal lymph node was harvested, stained with an anti-CD11b antibody and an anti-F4/80 antibody, and then analyzed for the uptake of the fluorescently labeled peptide into F4/80⁺CD11b⁺ cells by flow cytometry. The cells were analyzed in a flow cytometer (FACS Canto II, BD Biosciences) using attached analytical software (FACSDiva).

### (Example 6-1) Lymph Node Delivery of Various HA Derivatives

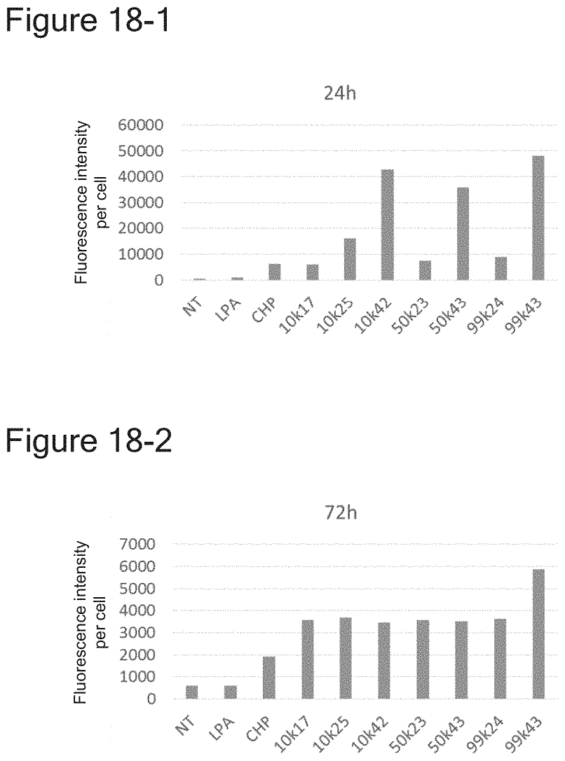

Each sample described in Table 18 was administered and analyzed by the method described in Example 6 to conduct a lymph node migration test. FIG. 18-1 shows fluorescence intensity per cell 24 hours later, and FIG. 18-2 shows fluorescence intensity per cell 72 hours later.

TABLE 18
Administration sample

| Abbre- | | Peptide species, | |
|---|---|---|---|
| viation | Carrier | Dose | Adjuvant |
| Peptide | None | Fluorescently labeled mERK2 p121 | None |
| | | 0.05 mg | |
| CHP | CHP-80T; | Fluorescently labeled mERK2 p121 | None |
| | 80k | 0.05 mg | |
| 10k17 | 10k HA-C₆- | Fluorescently labeled mERK2 p121 | None |
| | Chol-17% | 0.05 mg | |
| 10k25 | 10k HA-C₆- | Fluorescently labeled mERK2 p121 | None |
| | Chol-25% | 0.05 mg | |
| 10k42 | 10k HA-C₆- | Fluorescently labeled mERK2 p121 | None |
| | Chol-42% | 0.05 mg | |
| 50k23 | 50k HA-C₆- | Fluorescently labeled mERK2 p121 | None |
| | Chol-23% | 0.05 mg | |
| 50k43 | 50k HA-C₆- | Fluorescently labeled mERK2 p121 | None |
| | Chol-43% | 0.05 mg | |
| 99k24 | 99k HA-C₆- | Fluorescently labeled mERK2 p121 | None |
| | Chol-24% | 0.05 mg | |
| 99k43 | 99k HA-C₆- | Fluorescently labeled mERK2 p121 | None |
| | Chol-43% | 0.05 mg | |

The HA derivative group was shown to transfer more mERK2 to lymph node as compared with the peptide group and the CHP group.

### (Example 6-2) Lymph Node Delivery of Various Amino Acid-Modified HA Derivatives

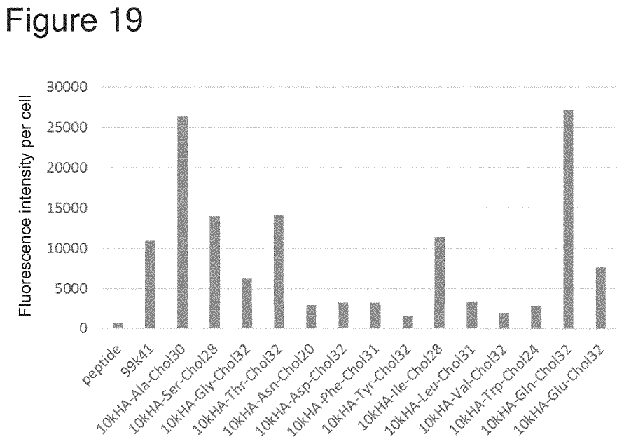

Each sample described in Table 19 was administered and analyzed by the method described in Example 6 to conduct a lymph node migration test. FIG. 19 shows fluorescence intensity per cell 24 hours later. The amino acid-modified HA derivatives were synthesized as described in Example 1 of International Publication No. WO 2014/038641. For example, 10kHA-Ala-C₆-Chol-30% of Table 19 below denotes a HA derivative of the formula (II) wherein R^{aa} is methyl (C₁ alkyl), R^{6a} is a hydrogen atom, X^{1a} is —NR⁹—Z¹—Z², R⁹ is a hydrogen atom, Z¹ is C₆ alkylene, Z² is —NR^{ba}—COO—Z³, R^{ba} is a hydrogen atom, and Z³ is a cholesteryl group (i.e., alanine and cholesteryl 6-aminohexyl carbamate were introduced), the weight-average molecular weight is 10 kDa; and the rate of introduction of the cholesteryl group is 30%. The synthesized amino acid-modified HA derivatives are described in Example 3-8 of International Publication No. WO 2014/038641.

TABLE 19
Administration sample

| | | Peptide species, | Ad- |
|---|---|---|---|
| Abbreviation | Carrier | Dose | juvant |
| Peptide | None | Fluorescently labeled MERK2 p121 | None |
| | | 0.05 mg | |
| 99k41 | 99k HA-C₆- | Fluorescently labeled MERK2 p121 | None |
| | Chol-41% | 0.05 mg | |
| 10kHA-Ala- | 10kHA-Ala- | Fluorescently labeled MERK2 p121 | None |
| Chol30 | C₆-Chol-30% | 0.05 mg | |
| 10kHA-Ser- | 10kHA-Ser- | Fluorescently labeled MERK2 p121 | None |
| Chol28 | C₆-Chol-28% | 0.05 mg | |
| 10kHA-Gly- | 10kHA-Gly- | Fluorescently labeled MERK2 p121 | None |
| Chol32 | C₆-Chol-32% | 0.05 mg | |
| 10kHA-Thr- | 10kHA-Thr- | Fluorescently labeled MERK2 p121 | None |
| Chol32 | C₆-Chol-32% | 0.05 mg | |
| 10kHA-Asn- | 10kHA-Asn- | Fluorescently labeled MERK2 p121 | None |
| Chol20 | C₆-Chol-20% | 0.05 mg | |
| 10kHA-Asp- | 10kHA-Asp- | Fluorescently labeled MERK2 p121 | None |
| Chol32 | C₆-Chol-32% | 0.05 mg | |
| 10kHA-Phe- | 10kHA-Phe- | Fluorescently labeled MERK2 p121 | None |
| Chol31 | C₆-Chol-31% | 0.05 mg | |
| 10kHA-Tyr- | 10kHA-Tyr- | Fluorescently labeled MERK2 p121 | None |
| Chol32 | C₆-Chol-32% | 0.05 mg | |
| 10kHA-Ile- | 10kHA-Ile- | Fluorescently labeled MERK2 p121 | None |
| Chol28 | C₆-Chol-28% | 0.05 mg | |
| 10kHA-Leu- | 10kHA-Leu- | Fluorescently labeled MERK2 p121 | None |
| Chol31 | C₆-Chol-31% | 0.05 mg | |
| 10kHA-Val- | 10kHA-Val- | Fluorescently labeled MERK2 p121 | None |
| Chol32 | C₆-Chol-32% | 0.05 mg | |
| 10kHA-Trp- | 10kHA-Trp- | Fluorescently labeled MERK2 p121 | None |
| Chol24 | C₆-Chol-24% | 0.05 mg | |
| 10kHA-Gln- | 10kHA-Gln- | Fluorescently labeled MERK2 p121 | None |
| Chol32 | C₆-Chol-32% | 0.05 mg | |
| 10kHA-Glu- | 10kHA-Glu- | Fluorescently labeled MERK2 p121 | None |
| Chol32 | C₆-Chol-32% | 0.05 mg | |

The amino acid-modified HA derivative group was shown to transfer mERK2 to lymph node.

### [Example 7] Mouse Tumor Growth Test

### (Example 7-1) Mouse Melanoma B16F10

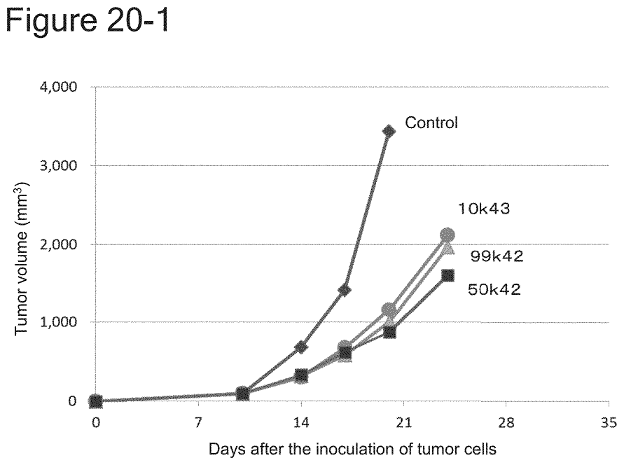

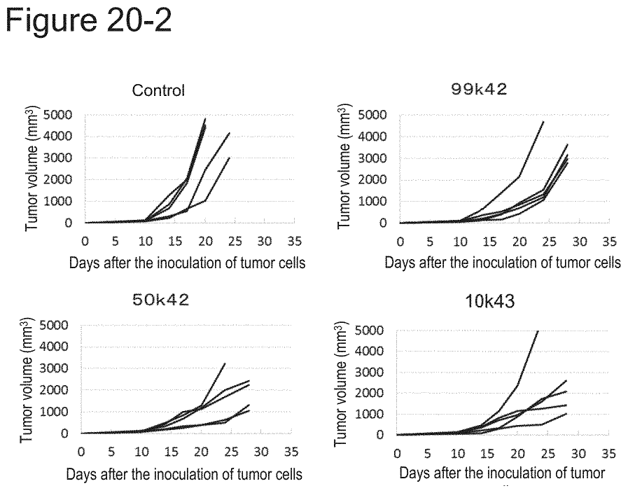

Each sample of Table 20 was subcutaneously administered with the frequency described in Table 20 to the mouse melanoma B16F10-bearing mice prepared by the method described in Example 5 to conduct a mouse tumor growth test. FIG. 20-1 shows changes in average tumor volume, and FIG. 20-2 shows changes in each individual.

TABLE 20
Administration sample

| | | Peptide | Adjuvant | Administration |
|---|---|---|---|---|
| Abbre- | | species, | species, | day (after |
| viation | Carrier | Dose | Dose | transplantation) |
| 99k42 | 99k HA-C₆- | TRP2TRP1gp100- | CpG | Day 10, day 14, |
| | Chol-42% | 6Y 0.05 mg | 0.05 mg | day 17, day 21 |
| 50k42 | 50k HA-C₆- | TRP2TRP1gp100- | CpG | Day 10, day 14, |
| | Chol-42% | 6Y 0.05 mg | 0.05 mg | day 17, day 21 |
| 10k43 | 10k HA-C₆- | TRP2TRP1gp100- | CpG | Day 10, day 14, |
| | Chol-43% | 6Y 0.05 mg | 0.05 mg | day 17, day 21 |

The HA derivative (50k42) group and the HA derivative (10k43) group, which differed in the molecular weight of HA from the HA derivative (99k42) group, were also shown to have a high tumor growth suppressive effect, as in the HA derivative (99k42) group.

### (Example 7-2) Mouse Melanoma B16F10

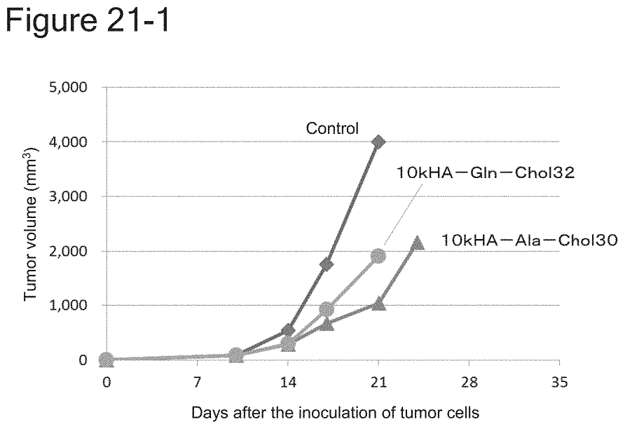

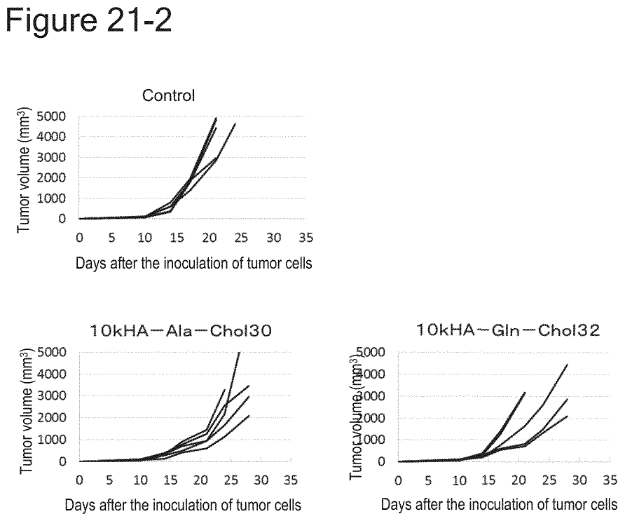

Each sample of Table 21 was subcutaneously administered with the frequency described in Table 21 to the mouse melanoma B16F10-bearing mice prepared by the method described in Example 5 to conduct a mouse tumor growth test. FIG. 21-1 shows changes in average tumor volume, and FIG. 21-2 shows changes in each individual.

TABLE 21
Administration sample

| | | Peptide | Adjuvant | Administration |
|---|---|---|---|---|
| Abbre- | | species, | species, | day (after |
| viation | Carrier | Dose | Dose | transplantation) |
| 10kHA- | 10kHA-Ala-C₆- | TRP2TRP1gp100- | CpG | Day 10, day 14, |
| Ala- | Chol-30% | 6Y 0.05 mg | 0.05 mg | day 17, day 20 |
| Chol30 | | | | |
| 10kHA- | 10kHA-Gln-C₆- | TRP2TRP1gp100- | CpG | Day 10, day 14, |
| Gln- | Chol-32% | 6Y 0.05 mg | 0.05 mg | day I 7, day 20 |
| Chol32 | | | | |

The amino acid-modified HA derivative groups (10kHA-Ala-Chol30 and 10kHA-Gln-Chol32) were also shown to have a high tumor growth suppressive effect.

### (Example 7-3) Mouse Melanoma B16F10

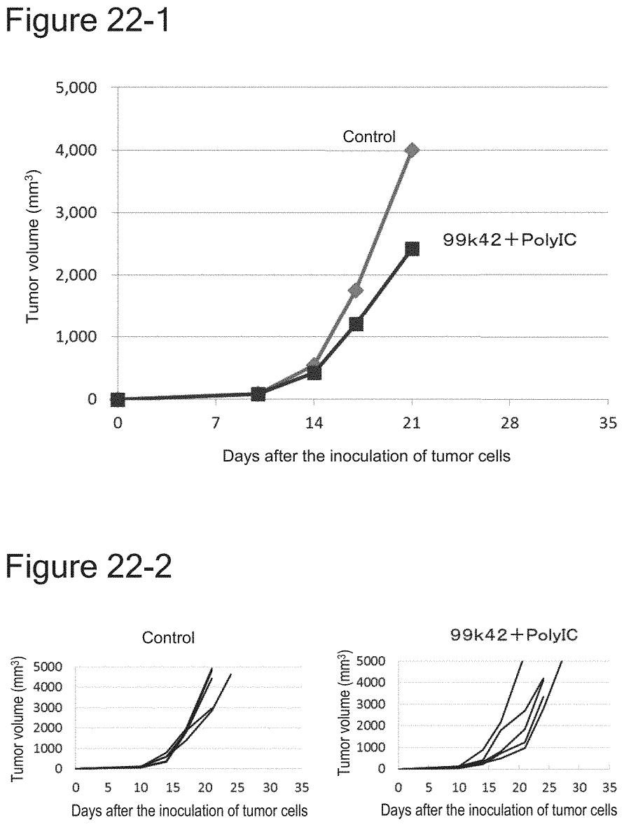

Each sample of Table 22 was subcutaneously administered with the frequency described in Table 22 to the mouse melanoma B16F10-bearing mice prepared by the method described in Example 5 to conduct a mouse tumor growth test. FIG. 22-1 shows changes in average tumor volume, and FIG. 22-2 shows changes in each individual.

TABLE 22
Administration sample

| | | Peptide | Adjuvant | Administration |
|---|---|---|---|---|
| Abbre- | | species, | species, | day (after |
| viation | Carrier | Dose | Dose | transplantation) |
| 99k42 + | 99k HA-C₆- | TRP2TRP1gp100- | PolyIC | Day 10, day 14, |
| PolyIC | Chol-42% | 6Y 0.05 mg | 0.05 mg | day 17, day 21 |

Use of polyIC as an adjuvant was also shown to produce a high tumor growth suppressive effect.

### (Example 7-4) Mouse Melanoma B16F10

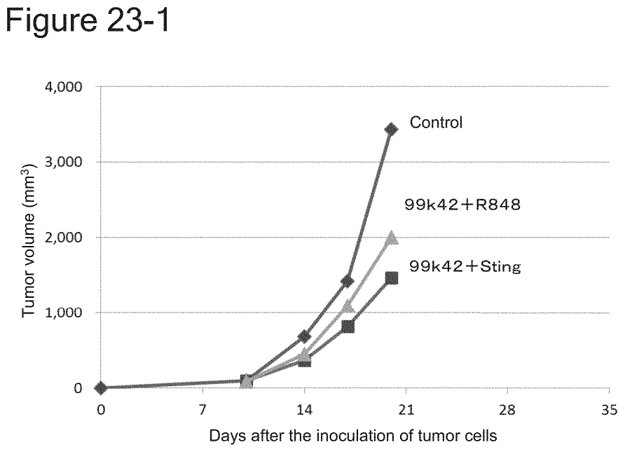

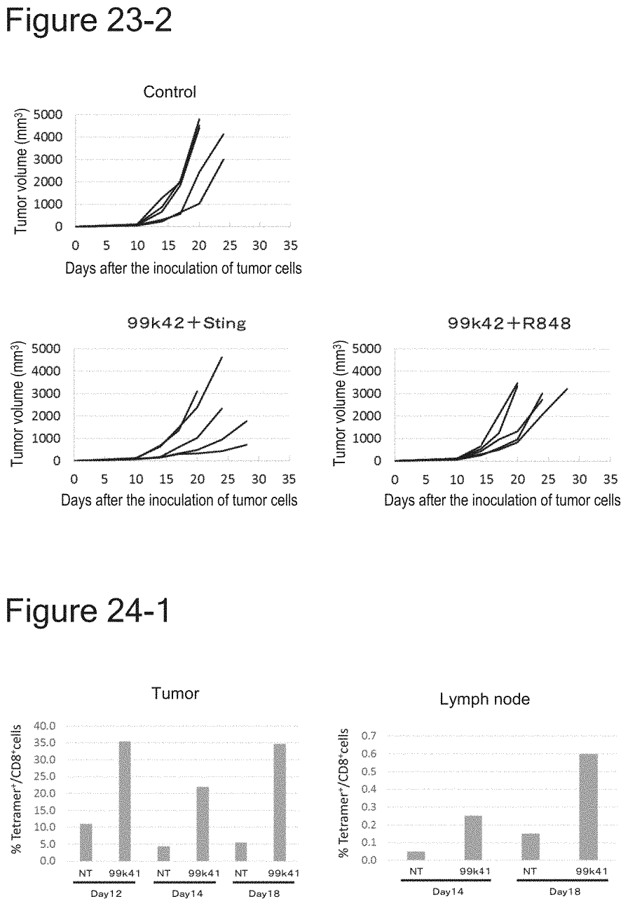

Each sample of Table 23 was subcutaneously administered with the frequency described in Table 23 to the mouse melanoma B16F10-bearing mice prepared by the method described in Example 5 to conduct a mouse tumor growth test. FIG. 23-1 shows changes in average tumor volume, and FIG. 23-2 shows changes in each individual.

TABLE 23
Administration sample

| | | Peptide | Adjuvant | Administration |
|---|---|---|---|---|
| Abbre- | | species, | species, | day (after |
| viation | Carrier | Dose | Dose | transplantation) |
| 99k42 + | 99k HA-C₆- | TRP2TRP1gp100- | Sting | Day 10, day 14, |
| Sting | Chol-42% | 6Y 0.05 mg | 0.015 mg | day 17, day 20 |
| 99k42 + | 99k HA-C₆- | TRP2TRP1gp100- | R848 | Day 10, day 14, |
| R848 | Chol-42% | 6Y 0.05 mg | 0.05 mg | day 17, day 20 |

Use of Sting or R848 as an adjuvant was also shown to produce a high tumor growth suppressive effect.

### [Example 8] Tumor and Lymph Node Analysis

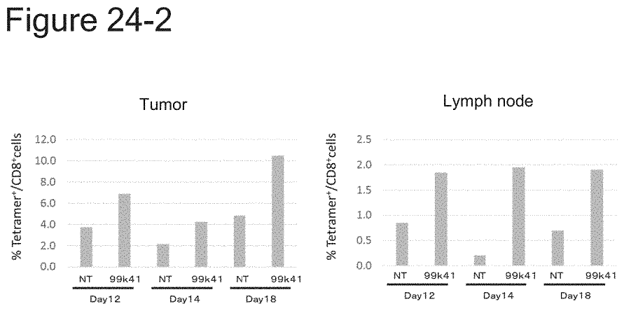

Each sample of Table 24 was subcutaneously administered with the frequency described in Table 24 to the mouse melanoma B16F10-bearing mice prepared by the method described in Example 5. On days 12, 14, and 18 after transplantation, tumor and lymph node (from a total of 4 locations of the right and left infra-axillary portions and the right and left inguinal portions) were harvested to conduct tetramer assay (mentioned later). The harvested tumor and lymph node from 2 animals were pooled, and two samples (from 4 animals) per group were analyzed. FIG. 24-1 shows the ratio of the number of TRP-2 antigen-specific CD8⁺ cells to the number of CD8⁺ cells, and FIG. 24-2 shows the ratio of the number of gp100 antigen-specific CD8⁺ cells to the number of CD8⁺ cells.

TABLE 24
Administration sample

| | | Peptide | Adjuvant | Administration |
|---|---|---|---|---|
| Abbre- | | species, | species, | day (after |
| viation | Carrier | Dose | Dose | transplantation) |
| 99k41 | 99k HA-C₆- | TRP2TRP1gp100- | CpG | Day 10, day 13, |
| | Chol-41% | 6Y 0.05 mg | 0.05 mg | day 17 |

It was shown that the TRP-2 antigen-specific CD8⁺ cells and the gp100 antigen-specific CD8⁺ cells invaded tumor and lymph node. This indicates that the antitumor effect brought about by the HA derivative is caused by antigen-specific CD8 cells.

Tetramer Assay

Each harvested tumor tissue sample was placed in a 6-well plate containing 1 mL/well of RPMI-1640, chopped into 2 mm square or smaller with scissors, and then recovered into gentleMACS C-tube (Miltenyi Biotech). After addition of Enzyme Mix (Miltenyi Biotech), the plate was loaded in gentleMACS Dissociator (Miltenyi Biotech), and the sample was disrupted. Then, the plate was incubated at 37° C. for 40 minutes and loaded again in gentleMACS Dissociator, and the sample was disrupted. The obtained cell suspension was passed through a strainer and then centrifuged (300×g, 5 min, 4° C.), and the pellets were suspended in MACS Buffer (Miltenyi Biotech) to prepare a cell suspension.

The harvested lymph node was mashed using the tail of the inner cylinder of a syringe to prepare a cell suspension.

5×10⁷ cells/mL of the tumor cell suspension or the lymph node cell suspension was added at 20 μL/well to a 96-well V-bottom microplate. A mouse FcR blocking reagent diluted 50-fold was added at 10 μL/well and reacted at 4° C. for 5 minutes. H-2K^{b} TRP-2 Tetramer-SVYDFFVWL-APC (Medical & Biological Laboratories Co., Ltd. (MBL)) or H-2D^{b} gp100 Tetramer-EGSRNQDWL-PE (MBL) was added at 10 μL/well and reacted at 4° C. for 30 minutes with light shielded. MACS Buffer was added at 200 μL/well, and a supernatant was removed by centrifugation (310 to 400×g, 5 min, 4° C.). This operation was repeated twice. Next, an antibody solution containing a fluorescently labeled anti-CD8 antibody (MBL) was added at 20 μL/well and reacted at 4° C. for 30 minutes with light shielded. MACS Buffer was added at 200 μL/well, and a supernatant was removed by centrifugation (310 to 400×g, 5 min, 4° C.). This operation was repeated twice. The cells were suspended by the addition of MACS Buffer at 20 μL/well, and analyzed in a flow cytometer (BD LSRFortessa X-20, BD Biosciences) using attached analytical software (FACSDiva).

1-14. (canceled)

15. A method for preventing and/or treating a cancer, comprising administering a vaccine formulation comprising a hyaluronic acid derivative having an introduced hydrophobic group and an antigen to a subject, wherein
the hyaluronic acid derivative having an introduced hydrophobic group is the following:
a hyaluronic acid derivative comprising at least one repeating unit represented by the formula (I): 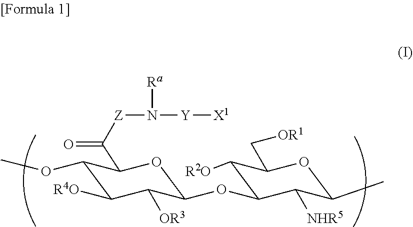
wherein R¹, R², R³, and R⁴ are each independently selected from a hydrogen atom, C₁₋₆ alkyl, formyl and C₁₋₆ alkylcarbonyl;
R⁵ is a hydrogen atom, formyl, or C₁₋₆ alkylcarbonyl;
Z represents a direct bond, or a peptide linker having 2 to 30 arbitrary amino acid residues;
X¹ is a hydrophobic group selected from groups represented by the following formulas:
—NR^{b}—R,
—NR^{b}—COO—R,
—NR^{b}—CO—R,
—NR^{b}—CO—NR^{c}—R,
—COO—R,
—O—COO—R,
—S—R,
—CO—Y^{a}—S—R,
—O—CO—Y^{b}—S—R,
—NR^{b}—CO—Y^{b}—S—R, and
—S—S—R;
R^{a}, R^{b} and R^{c} are each independently selected from a hydrogen atom, C₁₋₂₀ alkyl, amino-C₂₋₂₀ alkyl and hydroxy-C₂₋₂₀ alkyl, wherein in the alkyl moiety of each of the groups, 1 to 3 groups each independently selected from —O— and —NR^{f}— are optionally inserted;
R^{f} is selected from a hydrogen atom, C₁₋₁₂ alkyl, amino-C₂₋₁₂ alkyl and hydroxy-C₂₋₁₂ alkyl, wherein in the alkyl moiety of each of the groups, 1 or 2 groups each independently selected from —O— and —NH— are optionally inserted;
R is a steryl group;
Y is C₂₋₃₀ alkylene, or —(CH₂CH₂O)ₘ—CH₂CH₂—, wherein in the alkylene, 1 to 5 groups each independently selected from —O—, —NR^{g}— and —S—S— are optionally inserted;
R^{g} is selected from a hydrogen atom, C₁₋₂₀ alkyl, amino-C₂₋₂₀ alkyl or hydroxy-C₂₋₂₀ alkyl, wherein in the alkyl moiety of each of the groups, 1 to 3 groups each independently selected from —O— and —NH— are optionally inserted;
Y^{a} is C₁₋₅ alkylene;
Y^{b} is C₂₋₈ alkylene or C₂₋₈ alkenylene; and
m is an integer selected from 1 to 100; or
a hyaluronic acid derivative comprising a repeating unit represented by the formula (II): 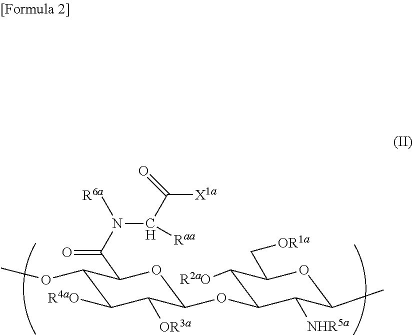
wherein R^{1a}, R^{2a}, R^{3a}, and R^{4a} are each independently selected from a hydrogen atom, C₁₋₆ alkyl, formyl, and C₁₋₆ alkylcarbonyl;
R^{a} is a hydrogen atom, formyl, or C₁₋₆ alkylcarbonyl;
X^{1a} is hydroxy, —O⁻Q⁺, C₁₋₆ alkoxy, —NR⁷R⁸, or —NR⁹—Z¹—Z²;
Q⁺ represents a counter cation;
R^{6a}, R⁷, R⁸, and R⁹ are each independently selected from a hydrogen atom, and C₁₋₆ alkyl;
R^{aa} is a hydrogen atom, or C₁₋₆ alkyl, wherein the alkyl is optionally substituted by one or more groups each independently selected from hydroxy, carboxy, carbamoyl, C₁₋₆ alkylthio, aryl, and heteroaryl, wherein the aryl is optionally substituted by one or more hydroxy groups;
Z¹ is C₂₋₃₀ alkylene, or —(CH₂CH₂O)ₘₐ—CH₂CH₂—, wherein in the alkylene, 1 to 5 groups each independently selected from —O—, —NR^{ga}— and —S—S— are optionally inserted, and ma is an integer selected from 1 to 100;
Z² is selected from groups represented by the following formulas:
—NR^{ba}—Z³,
—NR^{ba}—COO—Z³,
—NR^{ba}—CO—Z³,
—NR^{ba}—CO—NR^{ca}—Z³,
—COO—Z³,
—CO—NR^{ca}—Z³,
—O—CO—NR^{ca}—Z³,
—O—COO—Z³,
—S—Z³,
—CO—Z^{a}—S—Z³,
—O—CO—Z^{b}—S—Z³,
—NR^{ba}—CO—Z^{b}—S—Z³, and
—S—S—Z³;
R^{ba} and R^{ca} are each independently selected from a hydrogen atom, C₁₋₂₀ alkyl, amino-C₂₋₂₀ alkyl and hydroxy-C₂₋₂₀ alkyl, wherein in the alkyl moiety of each of the groups, 1 to 3 groups each independently selected from —O— and —NR^{fa}— are optionally inserted;
R^{fa} is independently selected from a hydrogen atom, C₁₋₁₂ alkyl, amino-C₂₋₁₂ alkyl and hydroxy-C₂₋₁₂ alkyl, wherein in the alkyl moiety of each of the groups, 1 or 2 groups each independently selected from —O— and —NH— are optionally inserted;
R^{ga} is independently selected from a hydrogen atom, C₁₋₂₀ alkyl, amino-C₂₋₂₀ alkyl and hydroxy-C₂₋₂₀ alkyl, wherein in the alkyl moiety of each of the groups, 1 to 3 groups each independently selected from —O— and —NH— are optionally inserted;
Z³ is a steryl group;
Z^{a} is C₁₋₅ alkylene; and
Z^{b} is C₂₋₈ alkylene or C₂₋₈ alkenylene,
the hyaluronic acid derivative, when not comprising a repeating unit represented by the formula (II) wherein X^{1a} is —NR⁹—Z¹—Z², further comprising a repeating unit represented by the formula (III): 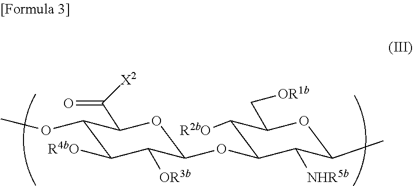
wherein R^{1b}, R^{2b}, R^{3b} and R^{4b} are each independently selected from a hydrogen atom, C₁₋₆ alkyl, formyl, and C₁₋₆ alkylcarbonyl;
R^{5b} is a hydrogen atom, formyl, or C₁₋₆ alkylcarbonyl; and
X² is —NR⁹—Z¹—Z², wherein R⁹, Z¹, and Z² are as already defined.

16. The method according to claim 15, wherein the hyaluronic acid derivative having an introduced hydrophobic group further comprises a repeating unit represented by the formula (IIIc): 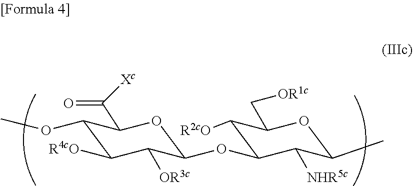
wherein R^{1c}, R^{2c}, R^{3c} and R^{4c} are each independently selected from a hydrogen atom, C₁₋₆ alkyl, formyl and C₁₋₆ alkylcarbonyl;
R^{5c} is selected from a hydrogen atom, formyl and C₁₋₆ alkylcarbonyl; and
X^{c} is selected from hydroxy and —O⁻Q⁺, wherein Q⁺ represents a counter cation.

17. The method according to claim 15, wherein the vaccine formulation comprises a hyaluronic acid derivative comprising a repeating unit represented by the formula (I), wherein a ratio of the repeating unit represented by the formula (I) to disaccharide repeating units present is 5 to 50%.

18. The method according to claim 15, wherein the vaccine formulation comprises a hyaluronic acid derivative comprising a repeating unit represented by the formula (II), wherein a ratio of a disaccharide unit comprising the group —NR⁹—Z¹—Z² to disaccharide repeating units present is 5 to 50%.

19. The method according to claim 15, wherein the vaccine formulation comprises a hyaluronic acid derivative comprising a repeating unit represented by the formula (I), wherein Z is a direct bond, Y is C₂₋₁₀ alkylene, X¹ is —NH—COO—R, and R is a cholesteryl group.

20. The method according to claim 15, wherein the vaccine formulation comprises a hyaluronic acid derivative comprising a repeating unit represented by the formula (II), wherein Z¹ is C₂₋₁₀ alkylene, Z² is —NH—COO—Z³, and Z³ is a cholesteryl group.

21. The method according to claim 15, wherein the hyaluronic acid derivative is produced using hyaluronic acid composed only of a disaccharide unit represented by the formula (IIIc) defined in claim 2, wherein when all of R^{1c}, R^{2c}, R^{3c}, and R^{4c} are hydrogen atoms, R^{5c} is acetyl, and X^{c} is —O⁻Na⁺, a weight-average molecular weight is 5 kilodaltons to 200 kilodaltons.

22. The method according to claim 15, wherein the hyaluronic acid derivative and the antigen form a complex.

23. The method according to claim 15, wherein the vaccine formulation is administered in combination with at least one type of adjuvant.

24. The method according to claim 15, wherein the antigen is an antigenic peptide or an antigenic protein.

25. The method according to claim 24, wherein the antigenic peptide comprises two or more CD8-positive cytotoxic T cell recognition epitopes or CD4-positive helper T cell recognition epitopes.

26. The method according to claim 25, wherein the antigenic peptide has an amino acid linker between the epitopes.

27. The method according to claim 15, wherein the vaccine formulation is administered in combination with at least one type of antibody for use in cancer treatment.

28. The method according to claim 15, wherein the vaccine formulation comprises a hyaluronic acid derivative comprising at least one repeating unit represented by the formula (I), wherein Y is —(CH₂)ₙ₁— or —(CH₂CH₂O)ₘ₁—CH₂CH₂—, n1 is an integer of 2 to 15, and m1 is an integer of 1 to 4.

29. The method according to claim 15, wherein the vaccine formulation is administered in combination with at least one type of adjuvant, and the adjuvant is i) a substance activating an innate immune receptor (pattern recognition receptor), ii) an antigen-presenting cell-stimulating substance, or iii) a substance having an effect of inhibiting the acquirement of immunosuppressive activity by antigen-presenting cells.

30. The method according to claim 15, wherein the antigen is an antigenic peptide, and the antigenic peptide comprises one or more each of CD8-positive cytotoxic T cell recognition epitopes and CD4-positive helper T cell recognition epitopes.

31. The method according to claim 15, wherein the antigen is an antigenic peptide, and the number of amino acid residues in the antigenic peptide is 8 to 120.

32. The method according to claim 15, wherein the antigen is an antigenic peptide, and the number of amino acid residues in the antigenic peptide is 8 to 50.

33. The method according to claim 15, wherein the vaccine formulation is administered in combination with at least one type of antibody for use in cancer treatment, and the antibody is an antibody inhibiting an immunosuppressive signal from tumor, or at least one type of antibody activating a costimulatory signal of immunocytes.

34. The method according to claim 15, wherein the vaccine formulation is administered in combination with at least one type of antibody for use in cancer treatment, and the antibody is an anti-CTLA4 antibody, an anti-PD1 antibody, an anti-PDL1 antibody, an anti-OX40 antibody, or an anti-4-1BB antibody.


SEQUENCE LISTING (23 sequences)

SEQ ID NO: 1 - protein, 37 aa, Artificial Sequence
  mERK2 p121
NDHIAYFLYQILRGLQYIHSANVLHRDLKPSNLLLNT

SEQ ID NO: 2 - protein, 9 aa, Artificial Sequence
  mERK2 p121 killer T cell epitope
QYIHSANVL

SEQ ID NO: 3 - protein, 17 aa, Artificial Sequence
  mERK2 p121 helper T cell epitope
RGLQYIHSANVLHRDLK

SEQ ID NO: 4 - protein, 40 aa, Artificial Sequence
  MAGE-A4 p264
GSNPARYEFLWGPRALAETSYVKVLEHVVRVNARVRIAYP

SEQ ID NO: 5 - protein, 9 aa, Artificial Sequence
  MAGE-A4 p264 killer T cell epitope
SNPARYEFL

SEQ ID NO: 6 - protein, 16 aa, Artificial Sequence
  MAGE-A4 p264 helper T cell epitope
VKVLEHVVRVNARVRI

SEQ ID NO: 7 - protein, 9 aa, Artificial Sequence
  MAGE-A4 p265
SNPARYEFL

SEQ ID NO: 8 - protein, 19 aa, Artificial Sequence
  MAGE-A4 p285
VKVLEHVVRVNARVRIAYP

SEQ ID NO: 9 - protein, 38 aa, Artificial Sequence
  TRP2TRP1gp100-6G
SVYDFFVWLGGGGGGTWHRYHLLGGGGGGEGSRNQDWL

SEQ ID NO: 10 - protein, 38 aa, Artificial Sequence
  TRP2TRP1gp100-6Y
SVYDFFVWLYYYYYYTWHRYHLLYYYYYYEGSRNQDWL

SEQ ID NO: 11 - protein, 34 aa, Artificial Sequence
  TRP2TRP1gp100-4L
SVYDFFVWLLLLLTWHRYHLLLLLLEGSRNQDWL

SEQ ID NO: 12 - protein, 38 aa, Artificial Sequence
  TRP2TRP1gp100-6L
SVYDFFVWLLLLLLLTWHRYHLLLLLLLLEGSRNQDWL

SEQ ID NO: 13 - protein, 9 aa, Artificial Sequence
  TRP2
SVYDFFVWL

SEQ ID NO: 14 - protein, 8 aa, Artificial Sequence
  TRP1
TWHRYHLL

SEQ ID NO: 15 - protein, 9 aa, Artificial Sequence
  gp100
EGSRNQDWL

SEQ ID NO: 16 - protein, 29 aa, Artificial Sequence
  AH1gp70-6G
LVQFIKDRISVVQAGGGGGGSPSYVYHQF

SEQ ID NO: 17 - protein, 29 aa, Artificial Sequence
  AH1gp70-6Y
LVQFIKDRISVVQAYYYYYYSPSYVYHQF

SEQ ID NO: 18 - protein, 27 aa, Artificial Sequence
  AH1gp70-4L
LVQFIKDRISVVQALLLLSPSYVYHQF

SEQ ID NO: 19 - protein, 29 aa, Artificial Sequence
  AH1gp70-6L
LVQFIKDRISVVQALLLLLLSPSYVYHQF

SEQ ID NO: 20 - protein, 9 aa, Artificial Sequence
  AH1
SPSYVYHQF

SEQ ID NO: 21 - protein, 14 aa, Artificial Sequence
  gp70
LVQFIKDRISVVQA

SEQ ID NO: 22 - protein, 40 aa, Artificial Sequence
  MAGE-A4 p264-4L
GSNPARYEFLWGPRALLLLLYVKVLEHVVRVNARVRIAYP

SEQ ID NO: 23 - protein, 40 aa, Artificial Sequence
  MAGE-A4 p264-4W
GSNPARYEFLWGPRALWWWWYVKVLEHVVRVNARVRIAYP